SEQUENCE LISTING (7 sequences)

SEQ ID NO: 1 - DNA, 3181 bp, Homo sapiens
  CDS (112)...(2886)
ccacgcgtccgcccacgcgtccgcggacgcgtgggcggacgcgtgggtgcgcgcagctcacaggccctgggagtgagctggtgcccggcgacctggcacccgcgcctggatatggggcgtctacatcgtcccaggagcagcaccagctacaggaacctgccgcatctgtttctgtttttcctcttcgtgggacccttcagctgcctcgggagttacagccgggccaccgagcttctgtacagcctaaacgagggactacccgcgggggtgctcatcggcagcctggccgaggacctgcggctgctgcccaggtctgcagggaggccggacccgcagtcgcagctgccagagcgcaccggtgctgagtggaacccccctctctccttcagcctggcctcccggggactgagtggccagtacgtgaccctagacaaccgctctggggagctgcacacttcagctcaggagatcgacagggaggccctgtgtgttgaagggggtggagggactgcgtggagcggcagcgtttccatctcctcctctccttctgactcttgtcttttgctgctggatgtgcttgtcctgcctcaggaatacttcaggtttgtgaaggtgaagatcgccatcagagacatcaatgacaacgccccgcagttccctgtttcccagatctcggtgtgggtcccggaaaatgcacctgtaaacacccgactggccatagagcatcctgctgtggacccagatgtaggcattaatggggtacagacctatcgcttactggactaccatggtatgttcaccctggacgtggaggagaatgagaatggggagcgcaccccctacctaattgtcatgggtgctttggacagggaaacccaggaccagtatgtgagcatcatcatagctgaggatggtgggtctccaccacttttgggcagtgccactctcaccattggcatcagtgacattaatgacaattgccctctcttcacagactcacaaatcaatgtcactgtgtatgggaatgctacagtgggcaccccaattgcagctgtccaggctgtggataaagacttggggaccaatgctcaaattacttattcttacagtcagaaagttccacaagcatctaaggatttatttcacctggatgaaaacactggagtcattaaacttttcagtaagattggaggaagtgttctggagtcccacaagctcaccatccttgctaatggaccaggctgcatccctgctgtaatcactgctcttgtgtccattattaaagttattttcagaccccctgaaattgtccctcgttacatagcaaacgagatagatggtgttgtttatctgaaagaactggaacccgttaacactcccattgcgtttttcaccataagagatccagaaggtaaatacaaggttaactgctacctggatggtgaagggccgtttaggttatcaccttacaaaccatacaataatgaatatttactagagaccacaaaacctatggactatgagctacagcagttctatgaagtagctgtggtggcttggaactctgagggatttcatgtcaaaagggtcattaaagtgcaacttttagatgacaatgataatgctccaattttccttcaacccttaatagaactaaccatcgaagagaacaactcacccaatgcctttttgactaagctgtatgctacagatgccgacagcgaggagagaggccaagtttcatattttctgggacctgatgctccatcatatttttccttagacagtgtcacaggaattctgacagtttctactcagctggaccgagaagagaaagaaaagtacagatacactgtcagagctgttgactgtgggaagccacccagagaatcagtagccactgtggccctcacagtgttggataaaaatgacaacagtcctcggtttatcaacaaggacttcagcttttttgtgcctgaaaactttccaggctatggtgagattggagtaattagtgtaacagatgctgacgctggacgaaatggatgggtcgccctctctgtggtgaaccagagtgatatttttgtcatagatacaggaaagggtatgctgagggctaaagtctctttggacagagagcagcaaagctcctatactttgtgggttgaagctgttgatgggggtgagcctgccctctcctctacagcaaaaatcacaattctccttctagatatcaatgacaaccctcctcttgttttgtttcctcagtctaatatgtcttatctgttagtactgccttctactctgccaggctccccggttacagaagtctatgctgtcgacaaagacacaggcatgaatgctgtcatagcttacagcatcatagggagaagaggtcctaggcctgagtccttcaggattgaccctaaaactggcaacattactttggaagaggcattgctgcagacagattatgggctccatcgcttactggtgaaagtgagtgatcatggttatcccgagcctctccactccacagtcatggtgaacctatttgtcaatgacactgtcagtaatgagagttacattgagagtcttttaagaaaagaaccagagattaatatagaggagaaagaaccacaaatctcaatagaaccgactcataggaaggtagaatctgtgtcttgtatgcccaccttagtagctctgtctgtaataagcttgggttccatcacactggtcacagggatgggcatatacatctgtttaaggaaaggggaaaagcatcccagggaagatgaaaatttggaagtacagattccactgaaaggaaaaattgacttgcatatgcgagagagaaagccaatggatatttctaatatttgatatttcatggtggaataacacagagaaatgttttaactgactttggatcttcatcacctaaaaaagagtgtgttgatggcagttccaatgaaggacaactaatttataacttgttctatattgtaaatagctgtttacaggtttttaaatttaaattcagaggttataaaatgtgtacagcatttttaagtgaaaattagtactaacagctataggacttgtatttaaaaaaaaaaaaaaaaaaagatctttaattaagcggccgcaagcttaaaccctttagtgagggttaatt

SEQ ID NO: 2 - protein, 924 aa, Homo sapiens [sequence omitted]

SEQ ID NO: 3 - DNA, 1967 bp, Homo sapiens
  CDS (1)...(1967)
atggggcgtctacatcgtcccaggagcagcaccagctacaggaacctgccgcatctgtttctgtttttcctcttcgtgggacccttcagctgcctcgggagttacagccgggccaccgagcttctgtacagcctaaacgagggactacccgcgggggtgctcatcggcagcctggccgaggacctgcggctgctgcccaggtctgcagggaggccggacccgcagtcgcagctgccagagcgcaccggtgctgagtggaacccccctctctccttcagcctggcctcccggggactgagtggccagtacgtgaccctagacaaccgctctggggagctgcacacttcagctcaggagatcgacagggaggccctgtgtgttgaagggggtggagggactgcgtggagcggcagcgtttccatctcctcctctccttctgactcttgtcttttgctgctggatgtgcttgtcctgcctcaggaatacttcaggtttgtgaaggtgaagatcgccatcagagacatcaatgacaacgccccgcagttccctgtttcccagatctcggtgtgggtcccggaaaatgcacctgtaaacacccgactggccatagagcatcctgctgtggacccagatgtaggcattaatggggtacagacctatcgcttactggactaccatggtatgttcaccctggacgtggaggagaatgagaatggggagcgcaccccctacctaattgtcatgggtgctttggacagggaaacccaggaccagtatgtgagcatcatcatagctgaggatggtgggtctccaccacttttgggcagtgccactctcaccattggcatcagtgacattaatgacaattgccctctcttcacagactcacaaatcaatgtcactgtgtatgggaatgctacagtgggcaccccaattgcagctgtccaggctgtggataaagacttggggaccaatgctcaaattacttattcttacagtcagaaagttccacaagcatctaaggatttatttcacctggatgaaaacactggagtcattaaacttttcagtaagattggaggaagtgttctggagtcccacaagctcaccatccttgctaatggaccaggctgcatccctgctgtaatcactgctcttgtgtccattattaaagttattttcagaccccctgaaattgtccctcgttacatagcaaacgagatagatggtgttgtttatctgaaagaactggaacccgttaacactcccattgcgtttttcaccataagagatccagaaggtaaatacaaggttaactgctacctggatggtgaagggccgtttaggttatcaccttacaaaccatacaataatgaatatttactagagaccacaaaacctatggactatgagctacagcagttctatgaagtagctgtggtggcttggaactctgagggatttcatgtcaaaagggtcattaaagtgcaacttttagatgacaatgataatgctccaattttccttcaacccttaatagaactaaccatcgaagagaacaactcacccaatgcctttttgactaagctgtatgctacagatgccgacagcgaggagagaggccaagtttcatattttctgggacctgatgctccatcatatttttccttagacagtgtcacaggaattctgacagtttctactcagctggaccgagaagagaaagaaaagtacagatacactgtcagagctgttgactgtgggaagccacccagagaatcagtagccactgtggccctcacagtgttggataaaaatgacaacagtcctcggtttatcaacaaggacttcagcttttttgtgcctgaaaactttccaggctatggtgagattggagtaattagtgtaacagatgctgacgctggacgaaatggatgggtcgccctctctgtggtgaaccagagtga

SEQ ID NO: 4 - DNA, 2938 bp, Homo sapiens
  CDS (162)...(2654)
ttcccgggtcgacccacgcgtccgccgcctacctgctcaagtgtccaccttgcctcgccccacctaagccaaatttgccagagctccctgaagaaggattcctttctcctggaaactggaccaagggagaggctttgggcatctgaaggtctgccttgaccatgatctctgcccggccgtggctactttacctctctgttattcaggctttcaccactgaggcccagcctgcagaaagcctgcacacagaagtccctgaaaactatggtggaaatttccctttttacatactcaagctaccactacccctggggagagatgaaggccacattgtcctatcaggagactcaaacacggcagatcaaaacacctttgctgtggacacagactctggctttctagtggcgacaaggaccctggaccgggaagagaaagcagaataccaactacaggtcaccttggagtctgaggatggacgtatcttgtggggtccacagcttgtgactgtgcatgtgaaagatgagaatgaccaggtaccccaattctcccaggccatctacagagctcagctgagccagggcaccaggcctggggtccccttcctcttccttgaggcttctgatggggatgcaccaggcacagctaactccgaccttcgcttccacattctgagccagtccccacctcagcctttaccagacatgttccagctggaccctcacctaggggctctggctcttagtcccagtggaagcaccagcctagaccatgcccttgaagagacttaccagctattggtacaggtcaaggacatgggtgaccagccttcaggccaccaggctattgcaactgtagagatctccatagtagagaacagctgggcacccctagagcctgttcacctggcagagaatctcaaagttgtgtacccacacagcattgcccaggtgcactggagtggaggagacgtgcactaccagctggagagccagcctccaggacccttcgatgtggatacagaggggatgctccatgttaccatggagctggaccgggaggcccaggctgagtaccagctccaagtccgagctcagaattcccatggtgaggactacgcagaacccctggagttgcaggtggtggtgatggatgaaaacgacaatgcacctgtctgctccccacatgacccaacagtcaacatccctgagctcagccccccaggaactgaaatagccaggctctcagcagaggatttggatgcccctgggtcacccaattcccacattgtatatcagttgttgagccctgagcctgaggagggggctgaaaacaaagccttcgagttagatccgacctcaggcagtgtaacactgggaactgccccactccatgctggccagagtatcctgcttcaggtgctggctgttgacctagcaggatcagagagtggcctcagcagcacatgtgaggtgacagtcatggtgacagacgtcaacaaccatgcccctgagttcatcaattcccagattgggcctgtaactcttcctgaggatgtaaaacctggggctctggtggcaacactcatggccactgatgctgaccttgaacctgccttccgccttatggactttgccattgaagaaggagacccagaagggatctttgacctgtcctgggagccagactccgaccatgtccagctcagactccggaagaacctcagctatgaggcagctcctgatcacaaggtggtggtggtcgtgagtaacatagaagaactggtgggcccaggcccaggccctgcagccacagccacagtgactatactagtggagagggtggttgctcccctcaagttggaccaggagagctatgagaccagcatcccagtcagcaccccagctggctccctcctgctgaccatccagccctcagaccccatgagcagaaccctcaggttctccctggtcaatgactcagagggctggctctgtatcaaggaggtgtctggggaggtacacacagcccagtccctgcagggtgcccagcctggagacacatacacagtgcttgtggaggcccaagacacagataagccaggactgagcacttctgccactgttgtgatccacttcctgaaggcctctcctgtcccagcattgactctgtctgctgggcccagccgacacctctgtacaccccgccaagactacggtgtagttgtgagtggggtcagtgaggaccctgacctagccaacaggaatggtccctacagctttgctctcggtcccaatcccactgtgcagcgggattggcgcctccagcctctcaacgattcccacgcctacctcaccttggcattgcattgggtagagcctggtgaatacatggtacctgtggttgtccaccatgatacccatatgtggcaactccaggtcaaagtgattgtgtgtcgctgcaacgtggaaggccaatgtatgcgcaaggtgggtcgcatgaagggaatgcccacgaaactgtcagcggtgggtgtcctcttgggcaccctggcagcgataggcttcattctcattcttgtgttcacgcacctggccctggcaaggaaggacctggatcagccagcagacagcgtgcctctgaaggcagcggtgtgaatgatccaagcagccccagctgggaggttggccccagctccctctgaactcactgagaaaggacccagtacccaagatgcactggggaccaagacagagtaaaagcccttcaccttgttggagtgaagacattatcacaggcatgtccccaaagcctgagcacctactttatgggatgaccatgggaacactccaaatggcagctctttgtccaataaaggctcagagagctagactggaaaaaaaaaaaaaaaaaaaaaaaaaaaaaaaaaaaaaaaaaaagg

SEQ ID NO: 5 - protein, 830 aa, Homo sapiens
MISARPWLLYLSVIQAFTTEAQPAESLHTEVPENYGGNFPFYILKLPLPLGRDEGHIVLSGDSNTADQNTFAVDTDSGFLVATRTLDREEKAEYQLQVTLESEDGRILWGPQLVTVHVKDENDQVPQFSQAIYRAQLSQGTRPGVPFLFLEASDGDAPGTANSDLRFHILSQSPPQPLPDMFQLDPHLGALALSPSGSTSLDHALEETYQLLVQVKDMGDQPSGHQAIATVEISIVENSWAPLEPVHLAENLKVVYPHSIAQVHWSGGDVHYQLESQPPGPFDVDTEGMLHVTMELDREAQAEYQLQVRAQNSHGEDYAEPLELQVVVMDENDNAPVCSPHDPTVNIPELSPPGTEIARLSAEDLDAPGSPNSHIVYQLLSPEPEEGAENKAFELDPTSGSVTLGTAPLHAGQSILLQVLAVDLAGSESGLSSTCEVTVMVTDVNNHAPEFINSQIGPVTLPEDVKPGALVATLMATDADLEPAFRLMDFAIEEGDPEGIFDLSWEPDSDHVQLRLRKNLSYEAAPDHKVVVVVSNIEELVGPGPGPAATATVTILVERVVAPLKLDQESYETSIPVSTPAGSLLLTIQPSDPMSRTLRFSLVNDSEGWLCIKEVSGEVHTAQSLQGAQPGDTYTVLVEAQDTDKPGLSTSATVVIHFLKASPVPALTLSAGPSRHLCTPRQDYGVVVSGVSEDPDLANRNGPYSFALGPNPTVQRDWRLQPLNDSHAYLTLALHWVEPGEYMVPVVVHHDTHMWQLQVKVIVCRCNVEGQCMRKVGRMKGMPTKLSAVGVLLGTLAAIGFILILVFTHLALARKDLDQPADSVPLKAAV

SEQ ID NO: 6 - DNA, 2493 bp, Homo sapiens
  CDS (1)...(2493)
atgatctctgcccggccgtggctactttacctctctgttattcaggctttcaccactgaggcccagcctgcagaaagcctgcacacagaagtccctgaaaactatggtggaaatttccctttttacatactcaagctaccactacccctggggagagatgaaggccacattgtcctatcaggagactcaaacacggcagatcaaaacacctttgctgtggacacagactctggctttctagtggcgacaaggaccctggaccgggaagagaaagcagaataccaactacaggtcaccttggagtctgaggatggacgtatcttgtggggtccacagcttgtgactgtgcatgtgaaagatgagaatgaccaggtaccccaattctcccaggccatctacagagctcagctgagccagggcaccaggcctggggtccccttcctcttccttgaggcttctgatggggatgcaccaggcacagctaactccgaccttcgcttccacattctgagccagtccccacctcagcctttaccagacatgttccagctggaccctcacctaggggctctggctcttagtcccagtggaagcaccagcctagaccatgcccttgaagagacttaccagctattggtacaggtcaaggacatgggtgaccagccttcaggccaccaggctattgcaactgtagagatctccatagtagagaacagctgggcacccctagagcctgttcacctggcagagaatctcaaagttgtgtacccacacagcattgcccaggtgcactggagtggaggagacgtgcactaccagctggagagccagcctccaggacccttcgatgtggatacagaggggatgctccatgttaccatggagctggaccgggaggcccaggctgagtaccagctccaagtccgagctcagaattcccatggtgaggactacgcagaacccctggagttgcaggtggtggtgatggatgaaaacgacaatgcacctgtctgctccccacatgacccaacagtcaacatccctgagctcagccccccaggaactgaaatagccaggctctcagcagaggatttggatgcccctgggtcacccaattcccacattgtatatcagttgttgagccctgagcctgaggagggggctgaaaacaaagccttcgagttagatccgacctcaggcagtgtaacactgggaactgccccactccatgctggccagagtatcctgcttcaggtgctggctgttgacctagcaggatcagagagtggcctcagcagcacatgtgaggtgacagtcatggtgacagacgtcaacaaccatgcccctgagttcatcaattcccagattgggcctgtaactcttcctgaggatgtaaaacctggggctctggtggcaacactcatggccactgatgctgaccttgaacctgccttccgccttatggactttgccattgaagaaggagacccagaagggatctttgacctgtcctgggagccagactccgaccatgtccagctcagactccggaagaacctcagctatgaggcagctcctgatcacaaggtggtggtggtcgtgagtaacatagaagaactggtgggcccaggcccaggccctgcagccacagccacagtgactatactagtggagagggtggttgctcccctcaagttggaccaggagagctatgagaccagcatcccagtcagcaccccagctggctccctcctgctgaccatccagccctcagaccccatgagcagaaccctcaggttctccctggtcaatgactcagagggctggctctgtatcaaggaggtgtctggggaggtacacacagcccagtccctgcagggtgcccagcctggagacacatacacagtgcttgtggaggcccaagacacagataagccaggactgagcacttctgccactgttgtgatccacttcctgaaggcctctcctgtcccagcattgactctgtctgctgggcccagccgacacctctgtacaccccgccaagactacggtgtagttgtgagtggggtcagtgaggaccctgacctagccaacaggaatggtccctacagctttgctctcggtcccaatcccactgtgcagcgggattggcgcctccagcctctcaacgattcccacgcctacctcaccttggcattgcattgggtagagcctggtgaatacatggtacctgtggttgtccaccatgatacccatatgtggcaactccaggtcaaagtgattgtgtgtcgctgcaacgtggaaggccaatgtatgcgcaaggtgggtcgcatgaagggaatgcccacgaaactgtcagcggtgggtgtcctcttgggcaccctggcagcgataggcttcattctcattcttgtgttcacgcacctggccctggcaaggaaggacctggatcagccagcagacagcgtgcctctgaaggcagcggtgtga

SEQ ID NO: 7 - protein, 11 aa, Homo sapiens
  VARIANT (1)...(1): Xaa=Leu, Iie or Val
XXXXDXNDXXP

(19) United States
(12) Patent Application Publication
(10) Pub. No.: US 2002/0081599 A1
(43) Pub. Date: Jun. 27, 2002

(54) 57809 and 57798, novel human cadherin molecules and uses therefor

(76) Inventor: Rory A. J. Curtis, Southborough, MA (US)

Correspondence Address: LAHIVE & COCKFIELD, 28 STATE STREET, BOSTON, MA, 02109, US

(21) Appl. No.: 09/838,529

(22) Filed: Apr. 18, 2001

Related U.S. Application Data
(60) Provisional application No. 60/198,466, filed on Apr. 18, 2000.

(51) Int. Cl. C12Q 1/68; C07H 21/04; C12P 21/02; C12N 5/06; C07K 14/575

(52) U.S. Cl. 435/6; 435/69.4; 435/325; 435/320.1; 536/23.5; 530/399

(57) ABSTRACT

The invention provides isolated nucleic acids molecules, designated CDHN nucleic acid molecules, which encode novel cadherin molecules. The invention also provides antisense nucleic acid molecules, recombinant expression vectors containing CDHN nucleic acid molecules, host cells into which the expression vectors have been introduced, and nonhuman transgenic animals in which a CDHN gene has been introduced or disrupted. The invention still further provides isolated CDHN proteins, fusion proteins, antigenic peptides and anti-CDHN antibodies. Diagnostic methods utilizing compositions of the invention are also provided.

### RELATED APPLICATIONS

This application claims priority to U.S. provisional patent application serial No. 60/198,466, filed Apr. 18, 2000. The contents of this provisional patent application are incorporated herein by reference in their entirety.

### BACKGROUND OF THE INVENTION

Cadherins form a superfamily of membrane glycoproteins which are involved in intercellular adhesion. The cadherin superfamily includes classical cadherins type 1 (e.g., E-cadherin) and type 2 (e.g., cadherin 11), desmosomal cadherins (e.g., desmogleins and desmocollins), and protocadherins (e.g., fat-like cadherins). Cadherins are important in forming cell junction adhesions, e.g., adherens junctions and desmosomes, and in the maintenance of cell-cell interactions. In addition to a role in cell adhesion, cadherins mediate signaling events that affect cell differentiation, proliferation, migration and survival.

Typically, cadherin molecules have three major regions, an extracellular domain that mediates specific adhesion, a transmembrane domain, and a cytoplasmic domain. The cytoplasmic domain serves to link cadherins to the cytoskeleton via a cadherin-associated complex (CAC), and to aggregate the cadherin proteins at sites of cell-cell attachment (Nagafuchi et al. (1989) Cell Reg. 1:37-44). Cadherin mediated cell adhesions are supported by the formation of lateral, cooperative cadherin cis dimers which are stabilized by attachment to the cytoskeleton, as well as the trans interactions in which they engage, e.g., homophilic interactions with cadherin molecules on apposed cells (Steinberg, M. S. et al. (1999) Curr. Opin. Cell Biol. 11:554-560). Cadherin mediated cell adhesion can be transiently modulated by the Rho family of small GTPases (e.g., rho, rac, cdc42) which regulate the actin cytoskeleton, as well as by tyrosine kinases and phosphatases (Tepass, U. (1999) Curr. Opin. Cell Biol. 11:540-548).

The cadherin cytoplasmic domain interacts with catenins (e.g., α, β and γ, p120^{ctn}), proteins that connect cadherins to the cytoskeleton, as well as other integral membrane proteins and peripheral cytoplasmic proteins (Steinberg, M. S. supra; Provost, E. et al. (1999) Curr. Opin. Cell Biol. 11:567-572). The catenin proteins are regulated by phosphorylation and may be involved in the modulation of cell proliferation and differentiation, as well as cell division. For example, β-catenin has an established role in the wnt signal transduction pathway in which it participates in the regulation of gene expression as a cotranscriptional regulator of the LEF/TCF family of transcription factors. Thus, cadherins are involved in signal transduction between the cell surface and the nucleus, and influence gene expression. Genetic analysis has revealed that β-catenin is involved in Xenopus and Drosophila embryonic development (e.g., in the establishment of dorsal-ventral and anterior-posterior axes), and acts as a protooncogene in may tumor types (Miller, J. R. et al. (1999) Oncogene 18:7860-7872; Tepass, U. supra).

Cell adhesion molecules are critical to the development of multi-cellular organisms. The spatio-temporal pattern of cadherin expression in developing tissues suggests an essential role in the establishment and maintenance of cell and tissue boundaries during differentiation, and in morphogenetic events such as adhesive contact formation, cell sorting, axonal patterning, neural plate induction, epithelial planar polarization, germ layer formation, organogenesis, and gastrulation (Tepass, U. supra). Alterations in cadherin expression or function correlates with morphoregulatory processes such as cell migration, cell differentiation and tissue rearrangement, as well as pathological states such as tumor formation and metastasis (Steinberg et al. supra; Behrens, J. (1999) Cancer Metastasis Rev. 18:15-30). Aberrant cadherin expression or function disrupts embryonic morphogenesis and may alter the characteristics of differentiated cells (Heasman et al. (1994) Development 120:49-57; Steinberg et al. supra; Behrens, J. supra).

### SUMMARY OF THE INVENTION

The present invention is based, at least in part, on the discovery of novel members of the family of cadherin molecules, referred to herein as “CDHN” nucleic acid and protein molecules (e.g., CDHN-1 and CDHN-2). The CDHN nucleic acid and protein molecules of the present invention are useful as modulating agents in regulating a variety of cellular processes, e.g., cellular proliferation, growth, adhesion, differentiation, or migration. Accordingly, in one aspect, this invention provides isolated nucleic acid molecules encoding CDHN proteins or biologically active portions thereof, as well as nucleic acid fragments suitable as primers or hybridization probes for the detection of CDHN-encoding nucleic acids.

In one embodiment, a CDHN nucleic acid molecule of the invention is at least 50%, 55%, 60%, 65%, 70%, 75%, 80%, 85%, 90%, 95%, 96%, 97%, 98%, 99% or more identical to the nucleotide sequence (e.g., to the entire length of the nucleotide sequence) shown in SEQ ID NO:1, 3, 4 or 6, or the nucleotide sequence of the DNA insert of the plasmid deposited with ATCC as Accession Number ______, or a complement thereof.

In a preferred embodiment, the isolated nucleic acid molecule includes the nucleotide sequence shown in SEQ ID NO:1, 3, 4 or 6, or a complement thereof. In another embodiment, the nucleic acid molecule includes SEQ ID NO:3 and nucleotides 1-111 of SEQ ID NO:1. In yet a further embodiment, the nucleic acid molecule includes SEQ ID NO:3 and nucleotides 2887-3181 of SEQ ID NO:1. In another preferred embodiment, the nucleic acid molecule consists of the nucleotide sequence shown in SEQ ID NO: 1 or 3. In another embodiment, the nucleic acid molecule includes SEQ ID NO:6 and nucleotides 1-161 of SEQ ID NO:4. In yet a further embodiment, the nucleic acid molecule includes SEQ ID NO:6 and nucleotides 2655-2938 of SEQ ID NO:4. In another preferred embodiment, the nucleic acid molecule consists of the nucleotide sequence shown in SEQ ID NO:4 or 6.

In another embodiment, a CDHN nucleic acid molecule includes a nucleotide sequence encoding a protein having an amino acid sequence sufficiently identical to the amino acid sequence of SEQ ID NO:2 or 5, or an amino acid sequence encoded by the DNA insert of the plasmid deposited with ATCC as Accession Number ______. In a preferred embodiment, a CDHN nucleic acid molecule includes a nucleotide sequence encoding a protein having an amino acid sequence at least 50%, 55%, 60%, 65%, 70%, 75%, 80%, 85%, 90%, 95%, 96%, 97%, 98%, 99% or more identical to the entire length of the amino acid sequence of SEQ ID NO:2 or 5, or the amino acid sequence encoded by the DNA insert of the plasmid deposited with ATCC as Accession Number ______.

In another preferred embodiment, an isolated nucleic acid molecule encodes the amino acid sequence of human CDHN-1 or human CDHN-2. In yet another preferred embodiment, the nucleic acid molecule includes a nucleotide sequence encoding a protein having the amino acid sequence of SEQ ID NO:2 or 5, or the amino acid sequence encoded by the DNA insert of the plasmid deposited with ATCC as Accession Number ______. In yet another preferred embodiment, the nucleic acid molecule is at least 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 2938, 3000, 3100, 3181 or more nucleotides in length. In a further preferred embodiment, the nucleic acid molecule is at least 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 2938, 3000, 3100, 3181 or more nucleotides in length and encodes a protein having a CDHN activity (as described herein).

Another embodiment of the invention features nucleic acid molecules, preferably CDHN nucleic acid molecules, which specifically detect CDHN nucleic acid molecules relative to nucleic acid molecules encoding non-CDHN proteins. For example, in one embodiment, such a nucleic acid molecule is at least 20, 30, 40, 50, 100, 150, 200, 250, 300, 350,400,450, 500,550,600,650, 700,750,800,850,900, 950, 1000, 1100, 1200, 1300, 1400, 1500, 1600, 1700, 1800, 1900, 2000, 2100, 2200, 2300, 2400, 2500, 2600, 2700, 2800, 2900, 3000 or more nucleotides in length and hybridizes under stringent conditions to a nucleic acid molecule comprising the nucleotide sequence shown in SEQ ID NO:1, 3, 4 or 6, the nucleotide sequence of the DNA insert of the plasmid deposited with ATCC as Accession Number ______, or a complement thereof.

In preferred embodiments, the nucleic acid molecules are at least 15 nucleotides (e.g., 15 contiguous nucleotides) in length and hybridize under stringent conditions to the nucleotide molecules set forth in SEQ ID NO:1, 3, 4 or 6.

In other preferred embodiments, the nucleic acid molecule encodes a naturally occurring allelic variant of a polypeptide comprising the amino acid sequence of SEQ ID NO:2 or 5, or an amino acid sequence encoded by the DNA insert of the plasmid deposited with ATCC as Accession Number, wherein the nucleic acid molecule hybridizes to a nucleic acid molecule comprising SEQ ID NO:1 or 3, or SEQ ID NO:4 or 6, respectively, under stringent conditions.

Another embodiment of the invention provides an isolated nucleic acid molecule which is antisense to a CDHN nucleic acid molecule, e.g., the coding strand of a CDHN nucleic acid molecule.

Another aspect of the invention provides a vector comprising a CDHN nucleic acid molecule. In certain embodiments, the vector is a recombinant expression vector. In another embodiment, the invention provides a host cell containing a vector of the invention. In yet another embodiment, the invention provides a host cell containing a nucleic acid molecule of the invention. The invention also provides a method for producing a protein, preferably a CDHN protein, by culturing in a suitable medium, a host cell, e.g., a mammalian host cell such as a non-human mammalian cell, of the invention containing a recombinant expression vector, such that the protein is produced.

Another aspect of this invention features isolated or recombinant CDHN proteins and polypeptides. In one embodiment, an isolated CDHN protein includes at least one or more of the following domains: a cadherin domain, a CA domain, a cadherins extracellular repeated domain signature pattern, a transmembrane domain, or a signal peptide. In a preferred embodiment, an isolated CDHN protein includes at least one, preferably two, three, four, five or more, cadherin domains. In another preferred embodiment, an isolated CDHN protein includes at least one, preferably two, three, four, five or more, cadherin domains, and at least one or more of the following domains: a CA domain, a cadherins extracellular repeated domain signature pattern, a transmembrane domain, or a signal peptide. In a further preferred embodiment, an isolated CDHN protein includes at least one, preferably two, three, four, five, or six CA domains. In another preferred embodiment, an isolated CDHN protein includes at least one, preferably two, three, four, five, or six CA domains, and at least one or more of the following domains: a cadherin domain, a cadherins extracellular repeated domain signature pattern, a transmembrane domain, or a signal peptide.

In a preferred embodiment, a CDHN protein includes at least one or more of the following domains: a cadherin domain, a CA domain, a cadherins extracellular repeated domain signature pattern, a transmembrane domain, or a signal peptide, and has an amino acid sequence at least about 50%, 55%, 60%, 65%, 67%, 68%, 70%, 75%, 80%, 85%, 90%, 95%, 96%, 97%, 98%, 99% or more identical to the amino acid sequence of SEQ ID NO:2 or 5, or the amino acid sequence encoded by the DNA insert of the plasmid deposited with ATCC as Accession Number______. In another preferred embodiment, a CDHN protein includes at least one or more of the following domains: a cadherin domain, a CA domain, a cadherins extracellular repeated domain signature pattern, a transmembrane domain, or a signal peptide, and has a CDHN activity (as described herein).

In yet another preferred embodiment, a CDHN protein includes at least one or more of the following domains: a cadherin domain, a CA domain, a cadherins extracellular repeated domain signature pattern, a transmembrane domain, or a signal peptide, and is encoded by a nucleic acid molecule having a nucleotide sequence which hybridizes under stringent hybridization conditions to a nucleic acid molecule comprising the nucleotide sequence of SEQ ID NO:1, 3, 4 or 6.

In another embodiment, the invention features fragments of the protein having the amino acid sequence of SEQ ID NO:2 or 5, wherein the fragment comprises at least 15 amino acids (e.g., contiguous amino acids) of the amino acid sequence of SEQ ID NO:2 or 5, or an amino acid sequence encoded by the DNA insert of the plasmid deposited with the ATCC as Accession Number ______. In another embodiment, a CDHN protein has the amino acid sequence of SEQ ID NO:2 or 5.

In another embodiment, the invention features a CDHN protein which is encoded by a nucleic acid molecule consisting of a nucleotide sequence at least about 50%, 55%, 60%, 65%, 70%, 75%, 80%, 85%, 90%, 95%, 96%, 97%, 98%, 99% or more identical to a nucleotide sequence of SEQ ID NO:1, 3, 4 or 6, or a complement thereof. This invention further features a CDHN protein which is encoded by a nucleic acid molecule consisting of a nucleotide sequence which hybridizes under stringent hybridization conditions to a nucleic acid molecule comprising the nucleotide sequence of SEQ ID NO:1, 3 4 or 6, or a complement thereof.

The proteins of the present invention or portions thereof, e.g., biologically active portions thereof, can be operatively linked to a non-CDHN polypeptide (e.g., heterologous amino acid sequences) to form fusion proteins. The invention further features antibodies, such as monoclonal or polyclonal antibodies, that specifically bind proteins of the invention, preferably CDHN proteins. In addition, the CDHN proteins or biologically active portions thereof can be incorporated into pharmaceutical compositions, which optionally include pharmaceutically acceptable carriers.

In another aspect, the present invention provides a method for detecting the presence of a CDHN nucleic acid molecule, protein, or polypeptide in a biological sample by contacting the biological sample with an agent capable of detecting a CDHN nucleic acid molecule, protein, or polypeptide such that the presence of a CDHN nucleic acid molecule, protein or polypeptide is detected in the biological sample.

In another aspect, the present invention provides a method for detecting the presence of CDHN activity in a biological sample by contacting the biological sample with an agent capable of detecting an indicator of CDHN activity such that the presence of CDHN activity is detected in the biological sample.

In another aspect, the invention provides a method for modulating CDHN activity comprising contacting a cell capable of expressing CDHN with an agent that modulates CDHN activity such that CDHN activity in the cell is modulated. In one embodiment, the agent inhibits CDHN activity. In another embodiment, the agent stimulates CDHN activity. In one embodiment, the agent is an antibody that specifically binds to a CDHN protein. In another embodiment, the agent modulates expression of a CDHN by modulating transcription of a CDHN gene or translation of a CDHN mRNA. In yet another embodiment, the agent is a nucleic acid molecule having a nucleotide sequence that is antisense to the coding strand of a CDHN mRNA or a CDHN gene.

In one embodiment, the methods of the present invention are used to treat a subject having a disorder characterized by aberrant or unwanted CDHN protein or nucleic acid expression or activity by administering an agent which is a CDHN modulator to the subject. In one embodiment, the CDHN modulator is a CDHN protein. In another embodiment the CDHN modulator is a CDHN nucleic acid molecule. In yet another embodiment, the CDHN modulator is a peptide, peptidomimetic, or other small molecule. In a preferred embodiment, the disorder characterized by aberrant or unwanted CDHN protein or nucleic acid expression is a cadherin-associated disorder, e.g., a central nervous system (CNS) disorder, a cardiovascular disorder, a musculoskeletal disorder, a gastrointestinal disorder, an inflammatory or immune system disorder, or a cell proliferation, growth, differentiation, adhesion, or migration disorder.

The present invention also provides diagnostic assays for identifying the presence or absence of a genetic alteration characterized by at least one of (i) aberrant modification or mutation of a gene encoding a CDHN protein; (ii) mis-regulation of the gene; and (iii) aberrant post-translational modification of a CDHN protein, wherein a wild-type form of the gene encodes a protein with a CDHN activity.

In another aspect the invention provides methods for identifying a compound that binds to or modulates the activity of a CDHN protein, by providing an indicator composition comprising a CDHN protein having CDHN activity, contacting the indicator composition with a test compound, and determining the effect of the test compound on CDHN activity in the indicator composition to identify a compound that modulates the activity of a CDHN protein.

Other features and advantages of the invention will be apparent from the following detailed description and claims.

### BRIEF DESCRIPTION OF THE DRAWINGS

FIG. 1 depicts the cDNA sequence and predicted amino acid sequence of human CDHN-1 (clone Fbh57798). The nucleotide sequence corresponds to nucleic acids 1 to 3181 of SEQ ID NO:1. The amino acid sequence corresponds to amino acids 1 to 924 of SEQ ID NO:2. The coding region without the 5′ and 3′ untranslated regions of the human CDHN-1 gene is shown in SEQ ID NO:3.

FIG. 2 depicts a structural, hydrophobicity, and antigenicity analysis of the human CDHN-1 protein (SEQ ID NO:2).

FIG. 3 depicts the results of a search which was performed against the MEMSAT database and which resulted in the identification of “transmembrane domains” in the human CDHN-1 protein (SEQ ID NO:2).

FIG. 4 depicts the results of a search which was performed against the HMM (PFAM) database and which resulted in the identification of “cadherin domains” in the human CDIN-1 protein (SEQ ID NO:2).

FIG. 5 depicts the results of a search which was performed against the HMM (SMART) database and which resulted in the identification of “CA” domains in the human CDHN-1 protein (SEQ ID NO:2).

FIG. 6 depicts the results of a search which was performed against the ProDom database and which resulted in the local alignment of the human CDHN-1 protein with p99.2 (671) FAT(32) Q14517(28) 088277(27); p99.2 (1) P81137_MANSE; p99.2 (1) O01909_CAEEL; p99.2 (1)O 93508_BRARE; and p99.2 (1) Q19319_CAEEL.

FIG. 7 depicts the cDNA sequence and predicted amino acid sequence of human CDHN-2 (clone Fbh57809). The nucleotide sequence corresponds to nucleic acids 1 to 2938 of SEQ ID NO:4. The amino acid sequence corresponds to amino acids 1 to 830 of SEQ ID NO:5. The coding region without the 5′ and 3′ untranslated regions of the human CDHN-2 gene is shown in SEQ ID NO:6.

FIG. 8 depicts a structural, hydrophobicity, and antigenicity analysis of the human CDHN-2 protein (SEQ ID NO:5).

FIG. 9 depicts the results of a search which was performed against the MEMSAT database and which resulted in the identification of “transmembrane domains” in the human CDHN-2 protein (SEQ ID NO:5).

FIG. 10 depicts the results of a search which was performed against the HMM (PFAM) database and which resulted in the identification of “cadherin domains” in the human CDHN-2 protein (SEQ ID NO:5).

FIG. 11 depicts the results of a search which was performed against the HMM (SMART) database and which resulted in the identification of “CA” domains in the human CDHN-2 protein (SEQ ID NO:5).

FIG. 12 depicts the results of a search which was performed against the ProDom database and which resulted in the local alignment of the human CDHN-2 protein with p99.2 (3) O75309(1) Q28634(1) O88338(1); p99.2 (1) O76356_CAEEL; p99.2 (1) Q19319_CAEEL; p99.2 (671) FAT (32) Q14517(28) O88277(27); p99.2 (1) P81137_MANSE; p99.2 (3) O75309(1) O88338(1) Q28634(1); p99.2 (1) ENDR_BOVIN; p99.2 (38) CAD1(4) DSC1(3) CAD2(3); p99.2 (3) CADL(1) Q12864(1) Q15336(1); and p99.2 (3) O75309(1) O88338(1) Q28634(1).

### DETAILED DESCRIPTION OF THE INVENTION

The present invention is based, at least in part, on the discovery of novel molecules, referred to herein as “cadherin” or “CDHN” nucleic acid and protein molecules, which are novel members of a family of cell adhesion molecules. These novel molecules are capable of mediating cell-cell and/or cell-substrate interactions. Thus, these novel CDHN molecules may play a role in or function in a variety of cellular processes, e.g., growth, proliferation, differentiation, adhesion, migration, signal transduction, cytoskeletal organization, transcriptional regulation, and inter- or intra-cellular communication.

As used herein, the term “cadherin” includes a molecule which is involved in cell-cell and/or cell-matrix adhesion. A variety of tissue-specific forms of cadherins have been identified including epithelial (E-cadherin), neural (N-cadherin), placental (P-cadherin), retinal (R-cadherin), vascular endothelial (VE-cadherin), kidney (K-cadherin), osteoblast (OB-cadherin), brain (BR-cadherin), muscle (M-cadherin) and liver-intestine (LI-cadherin), and cadherin subtype expression is correlated with the terminal differentiation of multiple cell types. Cadherin molecules have been shown to be involved in a variety of cellular adhesive events including cell sorting and patterning, multicellular organization, morphogenetic events during embryonic development, organogenesis, tissue remodeling, angiogenesis, tumorigeneis or metastasis. As cadherins, the CDHN molecules of the present invention provide novel diagnostic targets and therapeutic agents to control cadherin-associated disorders.

As used herein, a “cadherin-associated disorder” or a “CDHN associated disorder” includes a disorder, disease or condition which is caused or characterized by a misregulation (e.g., downregulation or upregulation) of a CDHN-mediated activity. Cadherin-associated disorders can detrimentally affect cellular functions such as cellular proliferation, growth, differentiation, adhesion, migration, or inter- or intra-cellular communication; tissue development, integrity and function, such as cardiac function, neuronal function, or musculoskeletal function. Examples of cadherin-associated disorders include central nervous system (CNS) disorders such as cognitive and neurodegenerative disorders, examples of which include, but are not limited to, Alzheimer's disease, dementias related to Alzheimer's disease (such as Pick's disease), Parkinson's and other Lewy diffuse body diseases, senile dementia, myasthenia gravis, Huntington's disease, Gilles de la Tourette's syndrome, multiple sclerosis, amyotrophic lateral sclerosis, progressive supranuclear palsy, epilepsy, and Jakob-Creutzfieldt disease; neurological developmental disorders such as neural tube defects, arrhinencephaly, spina bifida, adrenoleukodystrophy, Walker-Warburg syndrome, Miller-Dieker syndrome, Meckel-Gruber syndrome, meningomyelocele, Arnold-Chirai malformation, anencephaly, heterotopias, agyria, polymicrogyria, hydrocephalus, Zellweger syndrome, lissencephaly, cerebral palsy; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, autism, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Further CNS-related disorders include, for example, those listed in the American Psychiatric Association's Diagnostic and Statistical manual of Mental Disorders (DSM), the most current version of which is incorporated herein by reference in its entirety.

Further examples of cadherin-associated disorders include cardiac-related disorders. Cardiovascular system disorders in which the CDHN molecules of the invention may be directly or indirectly involved include arteriosclerosis, atherosclerosis, angiogenesis, ischemia reperfusion injury, restenosis, arterial inflammation, vascular wall remodeling, ventricular remodeling, coronary microembolism, coronary artery ligation, vascular heart disease, atrial fibrillation, congestive heart failure, sinus node dysfunction, angina, heart failure, hypertension, atrial fibrillation, cardiomyopathy, myocardial infarction, coronary artery disease, and arrhythmia; and cardiovascular developmental disorders (e.g., arteriovenous malformations, arteriovenous fistulae, raynaud's syndrome, neurogenic thoracic outlet syndrome, causalgia/reflex sympathetic dystrophy, hemangioma, aneurysm, cavernous angioma, aortic valve stenosis, atrial septal defects, atrioventricular canal, coarctation of the aorta, ebsteins anomaly, hypoplastic left heart syndrome, interruption of the aortic arch, mitral valve prolapse, ductus arteriosus, patent foramen ovale, partial anomalous pulmonary venous return, pulmonary atresia with ventricular septal defect, pulmonary atresia without ventricular septal defect, persistance of the fetal circulation, pulmonary valve stenosis, single ventricle, total anomalous pulmonary venous return, transposition of the great vessels, tricuspid atresia, truncus arteriosus, ventricular septal defects). CDHN mediated or related disorders also include disorders of the musculoskeletal system such as paralysis and muscle weakness, e.g., ataxia, myotonia, spinal muscle atrophy, myopathy, and myokymia; and musculoskeletal developmental disorders (e.g., cleft palate, midline skull defects, muscular dystrohies, Klippel-Feil syndrome).

CDHN disorders also include cellular proliferation, growth, differentiation, adhesion, or migration disorders. Cellular proliferation, growth, differentiation, adhesion, or migration disorders include those disorders that affect cell proliferation, growth, differentiation, adhesion, or migration processes. As used herein, a “cellular proliferation, growth, differentiation, adhesion, or migration process” is a process by which a cell increases in number, size or content, by which a cell develops a specialized set of characteristics which differ from that of other cells, or by which a cell moves closer to or further from a particular location or stimulus. The CDHN molecules of the present invention are involved in adhesive and signaling mechanisms which are known to be involved in cellular proliferation, growth, differentiation, adhesion, and migration processes. Thus, the CDHN molecules may modulate cellular proliferation, growth, differentiation, adhesion, or migration, and may play a role in disorders characterized by aberrantly regulated growth, differentiation, adhesion, or migration. Such disorders include cancer, e.g., carcinoma, sarcoma, lymphoma or leukemia, examples of which include, but are not limited to, breast, endometrial, ovarian, uterine, hepatic, gastrointestinal, prostate, colorectal, and lung cancer, melanoma, neurofibromatosis, adenomatous polyposis of the colon, Wilms' tumor, nephroblastoma, teratoma, rhabdomyosarcoma; tumor invasion, angiogenesis and metastasis; skeletal dysplasia; hematopoietic and/or myeloproliferative disorders.

CDHN-associated or related disorders also include inflammatory or immune system disorders, examples of which include, but are not limited to inflammatory bowel disease, ulcerative colitis, Crohn's disease, leukocyte adhesion deficiency II syndrome, peritonitis, chronic obstructive pulmonary disease, lung inflammation, asthma, nephritis, amyloidosis, rheumatoid arthritis, chronic bronchitis, sarcoidosis, scleroderma, lupus, polymyositis, Reiter's syndrome, psoriasis, pelvic inflammatory disease, inflammatory breast disease, orbital inflammatory disease, immune deficiency disorders (e.g., common variable immunodeficiency, congenital X-linked infantile hypogammaglobulinemia, transient hypogammaglobulinemia, selective IgA deficiency, chronic mucocutaneous candidiasis, severe combined immunodeficiency), wound healing, and autoimmune disorders (e.g., pemphigus vulgaris, paraneoplastic pemphigus).

A CDHN associated disorder also includes a hematopoietic or thrombotic disorder, for example, disseminated intravascular coagulation, thromboembolic vascular disease, anemia, lymphoma, leukemia, neutrophilia, neutropenia, myeloproliferative disorders, thrombocytosis, thrombocytopenia, von Willebrand disease, thalassaemia, and hemophilia.

In addition, CDHN associated disorders include gastrointestinal and digestive disorders including, but not limited to, esophageal disorders such as atresia and fistulas, stenosis, achalasia, esophageal rings and webs, hiatal hernia, lacerations, esophagitis, diverticulae, systemic sclerosis (scleroderma), varices, Barrett's esophagus, Mallory Weiss syndrome, esophageal tumors such as squamous cell carcinomas and adenocarcinomas, stomach disorders such as diaphragmatic hernias, pyloric stenosis, dyspepsia, gastritis, acute gastric erosion and ulceration, peptic ulcers, stomach tumors such as carcinomas and sarcomas, small intestine disorders such as congenital atresia and stenosis, diverticula, Meckel's diverticulum, Hirschsprung disease, pancreatic rests, insulin dependent diabetes mellitus, ischemic bowel disease, infective enterocolitis, Crohn's disease, tumors of the small intestine such as carcinomas and sarcomas, disorders of the colon such as malabsorption, obstructive lesions such as hernias, megacolon, diverticular disease, melanosis coli, ischemic injury, celiac disease, hemorrhoids, angiodysplasia of right colon, inflammations of the colon such as ulcerative colitis, tumors of the colon such as polyps and sarcomas, and abdominal wall defects; as well as hepatic disorders (e.g., cholestasis, cirrhosis, and hyperbilirubinemia) and renal disorders (e.g., renal failure, renal neoplasms, renal osteodystrophy, renal dysplasia, polycystic disease, and glomerulonephritis).

CDHN-associated or related disorders also include disorders affecting tissues in which CDHN (e.g., CDHN-1 or CDHN-2) protein is expressed. In one embodiment, a CDHN associated disorder is a disorder associated with aberrant cell patterning, differentiation and/or development in a tissue (e.g., an embryonic tissue) in which CDHN is expressed.

As used herein, a “cadherin-mediated activity” or a “CDHN-mediated activity” includes an activity which involves cadherin mediated adhesion or signal transduction. Cadherin-mediated activities include cell-cell and cell-matrix interactions, cell adhesion and migration, inter- and intra- cellular signaling.

The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin, as well as other, distinct proteins of human origin or alternatively, can contain homologues of non-human origin, e.g., monkey proteins. Members of a family may also have common functional characteristics.

A CDHN protein of the present invention includes a protein which comprises an extracellular domain, a transmembrane domain, and a cytoplasmic domain. In one embodiment, an extracellular domain of a CDHN protein may comprise at least one or more of the following domains: a cadherin domain, a CA domain, and/or a cadherins extracellular repeated domain signature pattern.

For example, the family of CDHN proteins comprises at least one “cadherin domain” in the protein or corresponding nucleic acid molecule. As used herein, the term “cadherin domain” includes a protein domain having an amino acid sequence of about 50-200 amino acid residues, preferably about 60-170 amino acid residues, more preferably about 70-140 amino acid residues, and more preferably about 80-110 amino acid residues, having a bit score for the alignment of the sequence to the cadherin domain (HMM) of at least about 14, more preferably 25, 27, 33, 40, 42, 49, 64, 75, 79 or greater. Cadherin domains are described in, for example, in Takeichi, M. (1990) Ann. Rev. Biochem., 59:237-252; Takeichi, M. (1987) Trends Genet., 3:213-217; and Mahoney et al. (1991) Cell, 67:853-868, the contents of which are incorporated herein by reference.

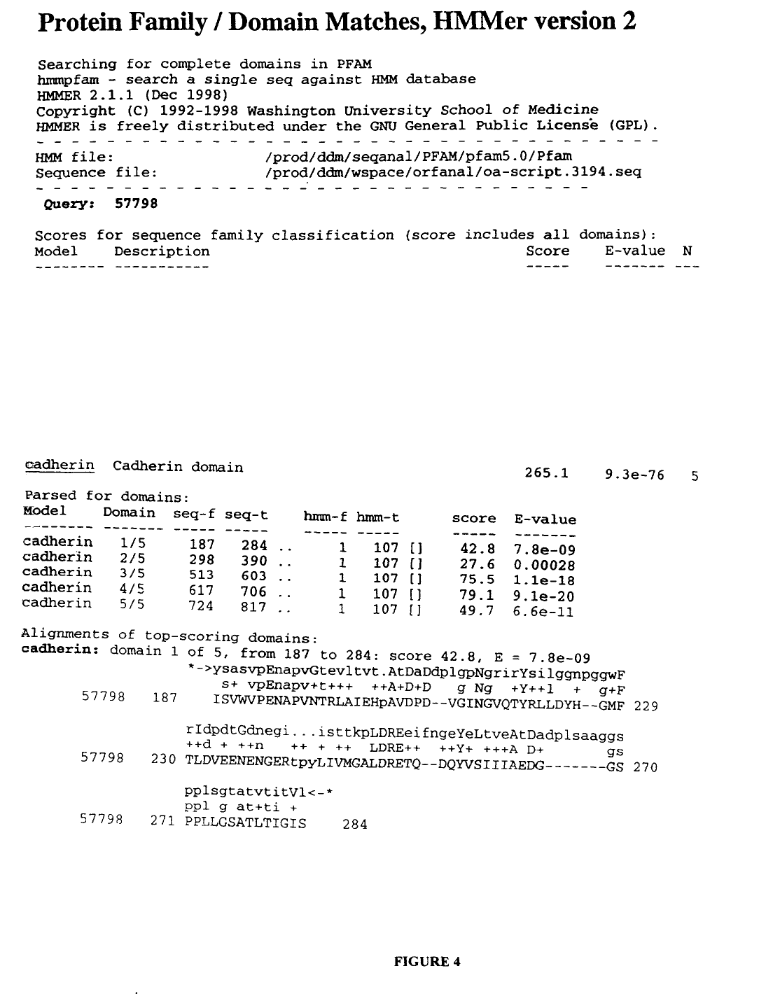

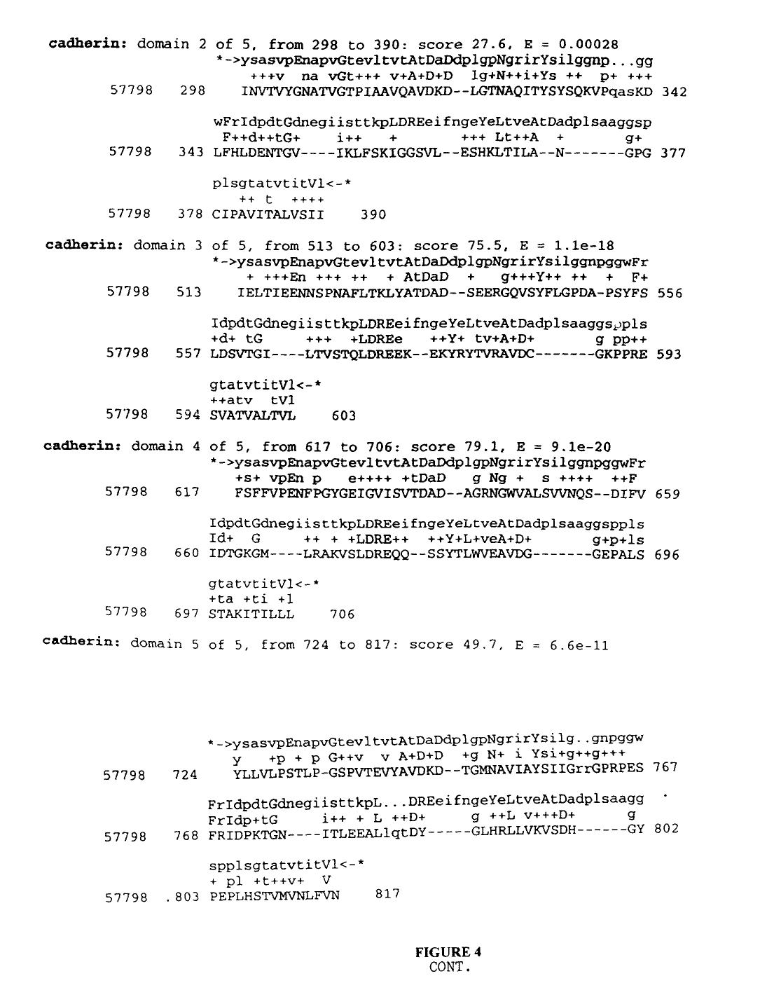

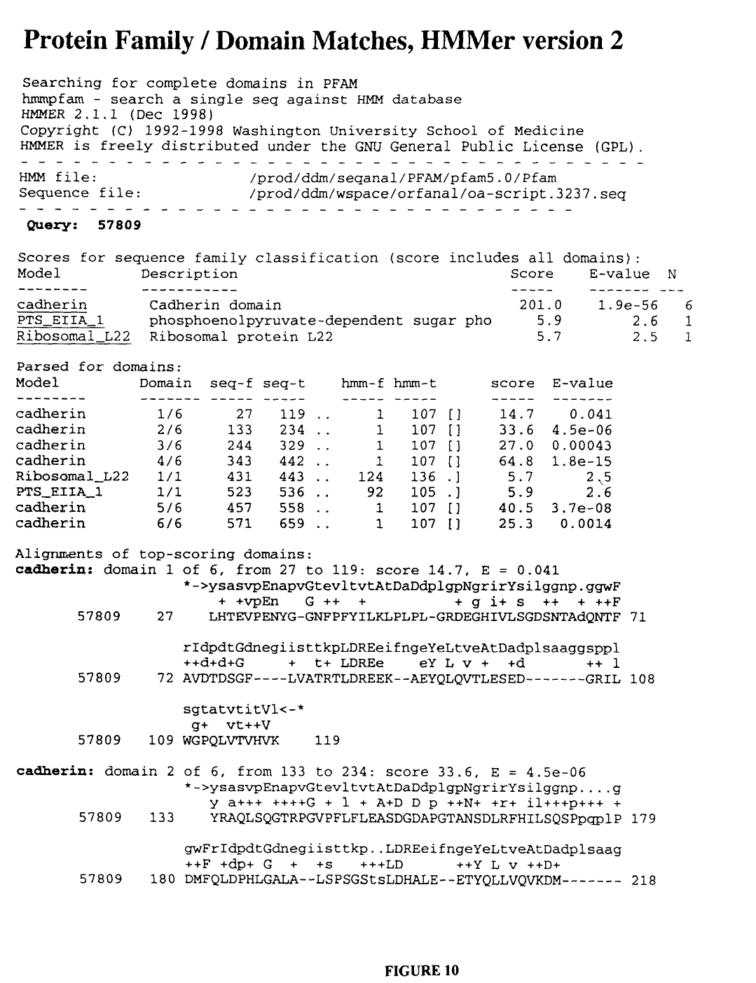

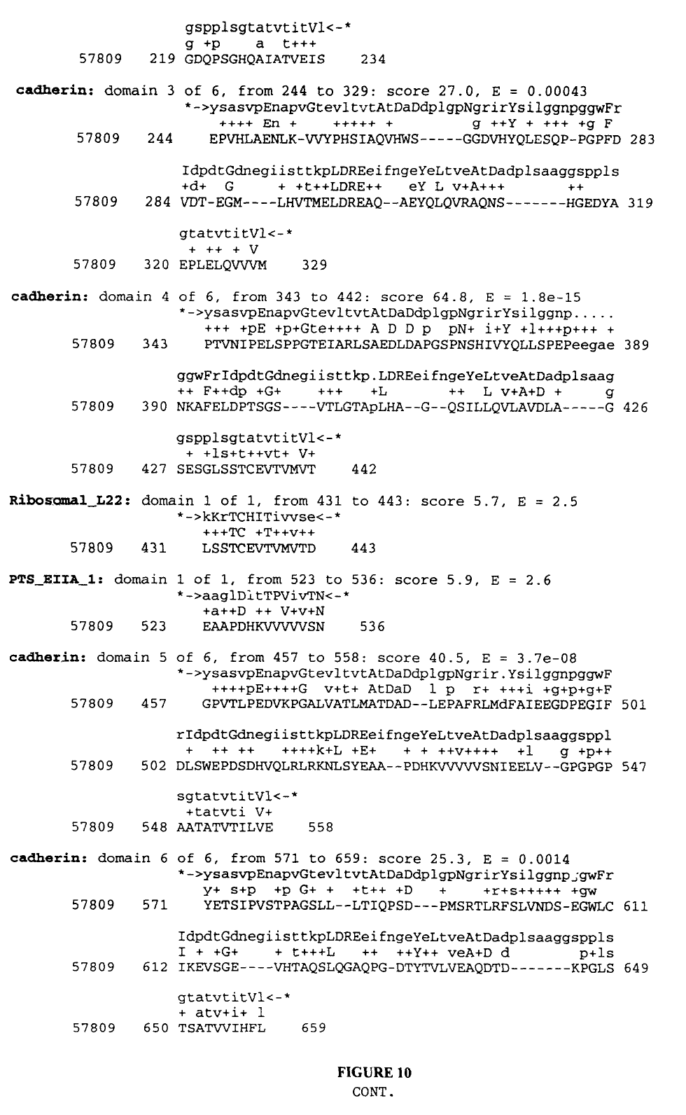

To identify the presence of a cadherin domain in a CDHN protein, and to make the determination that a protein of interest has a particular profile, the amino acid sequence of the protein is searched against a database of known protein domains (e.g., the HMM database). The cadherin domain (HMM) has been assigned the PFAM Accession PF00028 (http://genome.wustl.edu/Pfam/html). A search was performed against the HMM database resulting in the identification of cadherin domains in the amino acid sequence of human CDHN-1 at about residues 187-284, 298-390, 513-603, 617-706 and 724-817 of SEQ ID NO:2. The results of the search are set forth in FIG. 4. Cadherin domains were also identified in the amino acid sequence of human CDHN-2 at about residues 27-119, 133-234, 244-329, 343-442, 457-558 and 571-659 of SEQ ID NO:5. The results of the search are set forth in FIG. 10.

In one embodiment, a cadherin domain includes at least about 50-200 amino acid residues and has at least about 50-60% homology with a cadherin domain of human CDHN (e.g., residues 187-284, 298-390, 513-603, 617-706 and 724-817 of SEQ ID NO:2, or residues 27-119, 133-234, 244-329, 343-442, 457-558 and 571-659 of SEQ ID NO:5). Preferably, a cadherin domain includes at least about 70-140 amino acid residues, or about 80-110 amino acid residues, and has at least 60-70% homology, preferably about 70-80%, or about 80-90% homology with a cadherin domain of human CDHN (e.g., residues 187-284, 298-390, 513-603, 617-706 and 724-817 of SEQ ID NO:2, or residues 27-119, 133-234, 244-329, 343-442,457-558 and 571-659 of SEQ ID NO:5).

Accordingly, CDHN proteins having at least 50-60% homology, preferably about 60-70%, more preferably about 70-80%, or about 80-90% homology with a cadherin domain of human CDHN are within the scope of the invention.

In another embodiment, a CDHN protein of the present invention is identified based on the presence of at least one “CA domain” or “cadherin repeat domain” in the protein or corresponding nucleic acid molecule. As used herein, the term “CA domain” or “cadherin repeat domain” includes a protein domain having an amino acid sequence of about 40-130 amino acid residues, preferably about 50-120 amino acid residues, more preferably about 60-110 amino acid residues, and more preferably about 70-100 amino acid residues, having a bit score for the alignment of the sequence to the CA domain (HMM) of at least about 2, more preferably 6, 10, 23, 35, 45, 57, 58, 66, 67, 75, 85, 99, 103 or greater. Cadherin repeat domains are described in, for example, in Yap, AS. et al. (1997) Ann. Rev. Cell. Dev. Biol., 1:119-146; Overduin, M. et al. (1995) Science 267: 386-389; Shapiro, L. et al. (1995) Nature 374: 327-337; Shapiro, L. et al. (1995) Proc. Natl. Acad. Sci. USA 92: 6793-6797; and Takeichi, M. (1988) Development 102: 639-655, the contents of which are incorporated herein by reference.

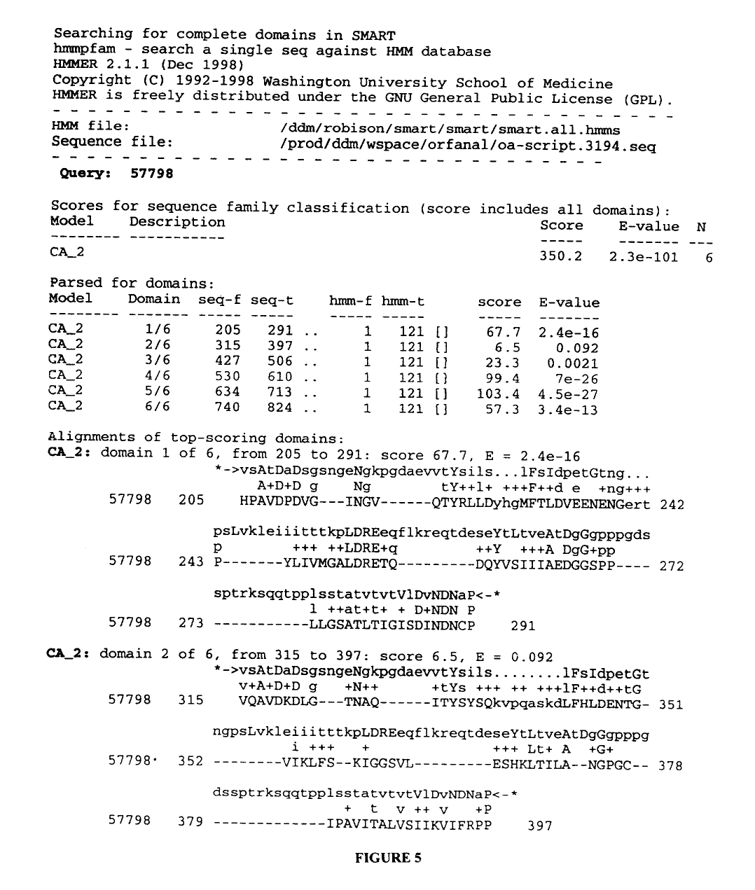

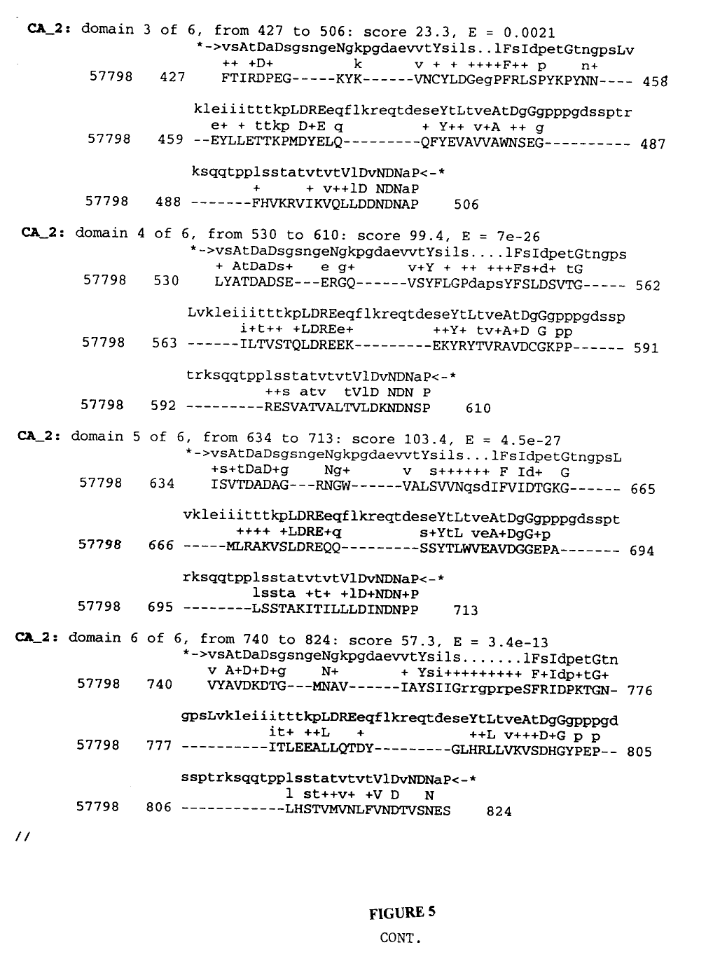

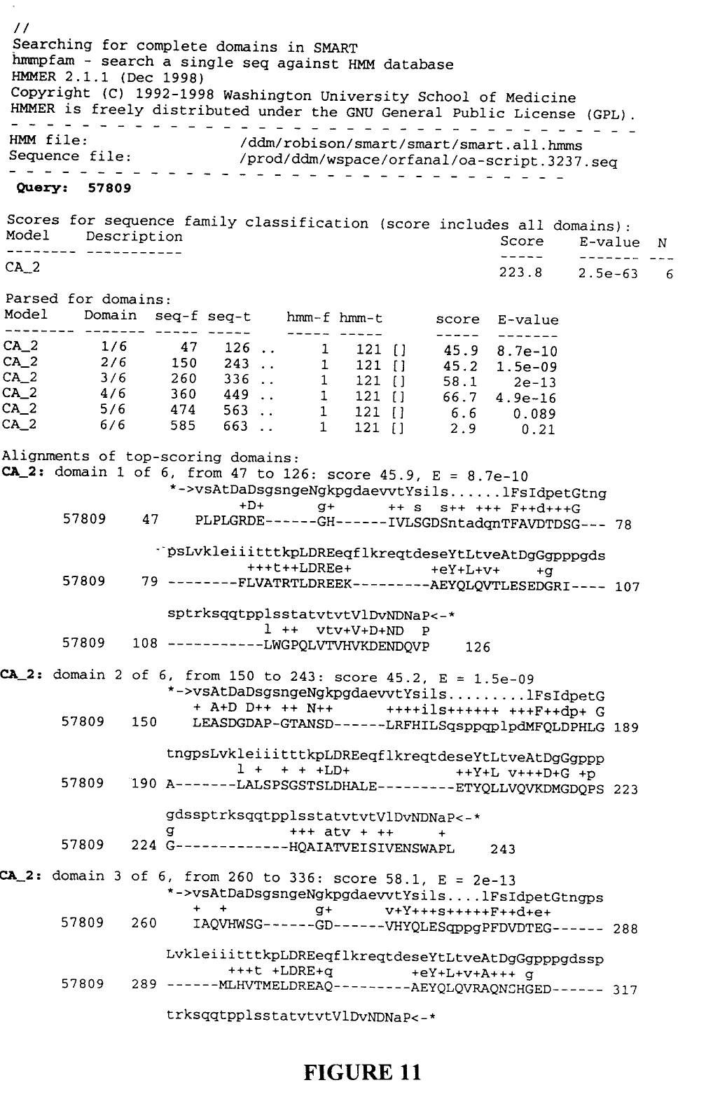

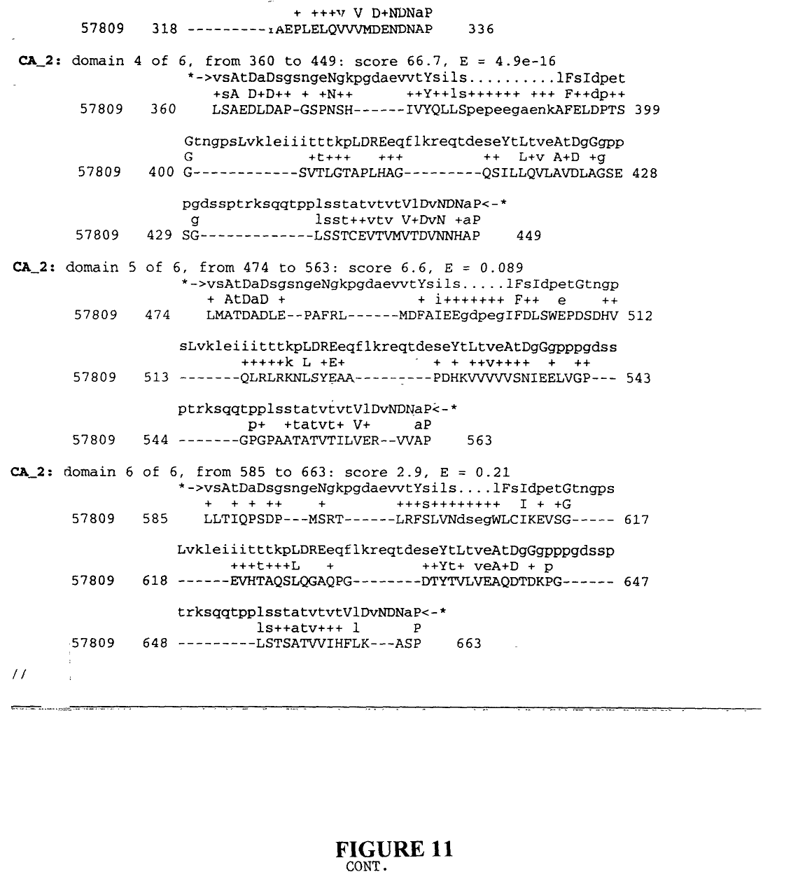

To identify the presence of a CA domain in a CDHN protein, and to make the determination that a protein of interest has a particular profile, the amino acid sequence of the protein is searched against a database of known protein domains (e.g., the HMM database). The CA domain (HMM) has been assigned the Prosite Profile PS50268 (http://smart.embl-heidelberg.de). A search was performed against the HMM database resulting in the identification of CA domains in the amino acid sequence of human CDHN-1 at about residues 205-291, 215-397, 427-506, 530-610, 634-713 and 740-824 of SEQ ID NO:2. The results of the search are set forth in FIG. 5. CA domains were also identified in the amino acid sequence of human CDHN-2 at about residues 47-126, 150-243, 260-336, 360-449, 474-563 and 585-663 of SEQ ID NO:5. The results of the search are set forth in FIG. 11.

In one embodiment, a CA domain includes at least about 40-130 amino acid residues and has at least about 50-60% homology with a CA domain of human CDHN (e.g., residues 205-291, 215-397, 427-506, 530-610, 634-713 and 740-824 of SEQ ID NO:2, or residues 47-126, 150-243, 260-336, 360-449, 474-563 and 585-663 of SEQ ID NO:5). Preferably, a CA domain includes at least about 60-110 amino acid residues, or about 70-100 amino acid residues, and has at least 60-70% homology, preferably about 70-80%, or about 80-90% homology with a CA domain of human CDHN (e.g., residues 205-291, 215-397, 427-506, 530-610, 634-713 and 740-824 of SEQ ID NO:2, or residues 47-126, 150-243, 260-336, 360-449, 474-563 and 585-663 of SEQ ID NO:5).

Accordingly, CDHN proteins having at least 50-60% homology, preferably about 60-70%, more preferably about 70-80%, or about 80-90% homology with a CA domain of human CDHN are within the scope of the invention.

In one embodiment, a CDHN protein comprises the following cadherins extracellular repeated domain signature pattern:

| | |
|---|---|
| [LIV]-X-[LIV]-X-D-X-N-D-[NH]-X-P | (SEQ ID NO:7) |

The signature patterns or consensus patterns described herein are described according to the following designation: all amino acids are indicated according to their universal single letter designation; “X” designates any amino acid; X(n) designates n number of amino acids, e.g., X (2) designates any two amino acids, e.g., X (1-3) designates any of one to three amino acids; and, amino acids in brackets indicates any one of the amino acids within the brackets, e.g., [LIV] indicates any of one of either L (leucine), I (isoleucine), or V (valine). Cadherins extracellular repeated domain signatures comprise asparagine residues, as well as conserved aspartic acid residues. In one embodiment the residues within the cadherins extracellular repeated domain signature pattern of SEQ ID NO:7 may be important for the binding of calcium.

To identify the presence of a cadherins extracellular repeated domain signature pattern in a CDHN protein, and to make the determination that a protein of interest has a particular profile, the amino acid sequence of the protein is searched against a database of known protein domains. The cadherins extracellular repeated domain signature pattern has been assigned the Prosite Accession Number PS00232 (www.expasy.ch/prosite). CDHN-1 has such a signature pattern at about amino acid residues 170-180, 281-291, 496-506, 600-610 and 703-713 of SEQ ID NO:2. CDHN-2 has such a signature pattern at about amino acid residues 326-336 of SEQ ID NO:5.

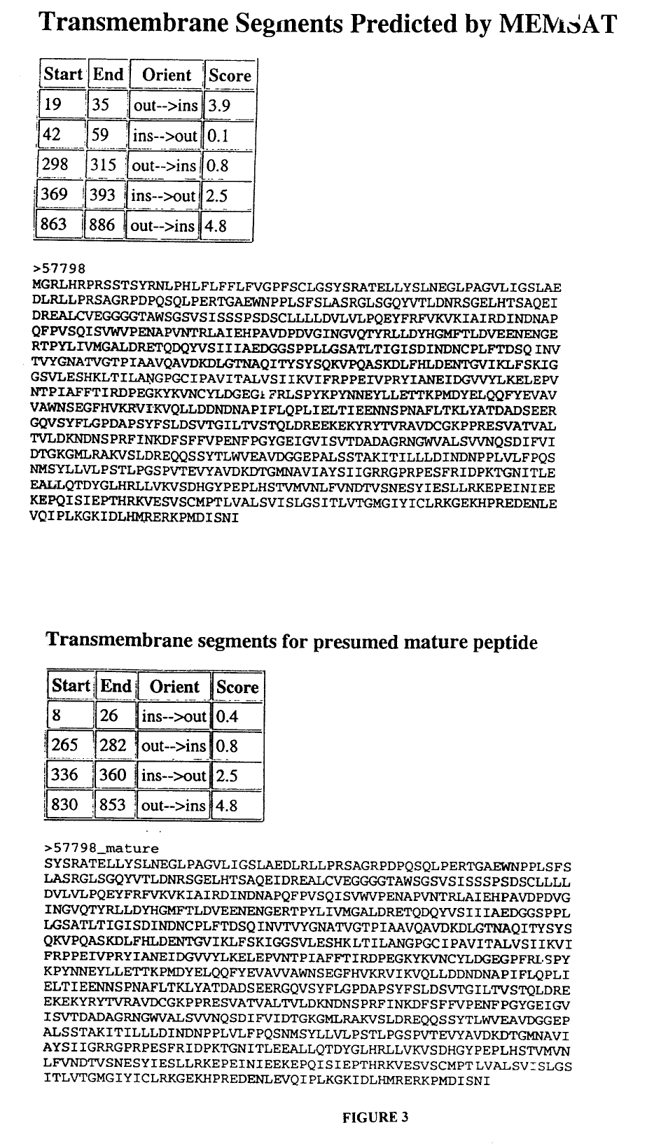

In another embodiment, a CDHN protein of the present invention is identified based on the presence of at least one “transmembrane domain”. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al., (1996) Annual Rev. Neurosci. 19: 235-263, the contents of which are incorporated herein by reference. Amino acid residues 19-35, 42-59, 298-315, 369-393 and 863-886 of the native CDHN-1 protein, and amino acid residues 8-26, 265-282, 336-360, 830-853 of the putative mature CDHN-1 protein are predicted to comprise a transmembrane domain (see FIG. 3). In addition, amino acid residues 540-557, 571-588 and 789-813 of the native CDHN-2 protein, and amino acid residues 519-536, 550-567 and 768-792 of the putative mature CDHN-2 protein are predicted to comprise a transmembrane domain (see FIG. 9). Accordingly, CDHN proteins having at least 50-60% homology, preferably about 60-70%, more preferably about 70-80%, or about 80-90% homology with a transmembrane domain of human CDHN are within the scope of the invention.

In another embodiment of the invention, a CDHN protein of the present invention is identified based on the presence of a signal peptide. The prediction of such a signal peptide can be made, for example, utilizing the computer algorithm SignalP (Henrik, et al. (1997) Protein Engineering 10:1-6). As used herein, a “signal sequence” or “signal peptide” includes a peptide containing about 15 or more amino acids which occurs at the N-terminus of secretory and membrane bound proteins and which contains a large number of hydrophobic amino acid residues. For example, a signal sequence contains at least about 10-30 amino acid residues, preferably about 15-25 amino acid residues, more preferably about 18-20 amino acid residues, and more preferably about 19 amino acid residues, and has at least about 35-65%, preferably about 38-50%, and more preferably about 40-45% hydrophobic amino acid residues (e.g., Valine, Leucine, Isoleucine or Phenylalanine). Such a “signal sequence”, also referred to in the art as a “signal peptide”, serves to direct a protein containing such a sequence to a lipid bilayer, and is cleaved in secreted and membrane bound proteins. A signal sequence was identified in the amino acid sequence of human CDHN-1 at about amino acids 1-33 of SEQ ID NO:2. A signal sequence was also identified in the amino acid sequence of human CDHN-2 at about amino acids 1-21 of SEQ ID NO:5. Accordingly, the present invention provides a mature CDHN protein lacking the signal peptide, e.g. amino acid residues 34-924 of SEQ ID NO:2 (CDHN-1) or amino acid residues 22-830 of SEQ ID NO:5 (CDHN-2).

In a preferred embodiment, the CDHN molecules of the invention include at least one or more of the following domains: a cadherin domain, a CA domain, a cadherins extracellular repeated domain signature pattern, a transmembrane domain, or a signal peptide.

Isolated proteins of the present invention, preferably CDHN proteins, have an amino acid sequence sufficiently identical to the amino acid sequence of SEQ ID NO:2 or 5, or are encoded by a nucleotide sequence sufficiently identical to SEQ ID NO:1, 3, 4 or 6. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 30%, 40%, or 50% homology, preferably 60% homology, more preferably 70%-80%, and even more preferably 90-95% homology across the amino acid sequences of the domains and contain at least one and preferably two structural domains or motifs, are defined herein as sufficiently identical. Furthermore, amino acid or nucleotide sequences which share at least 30%, 40%, or 50%, preferably 60%, more preferably 70-80%, or 90-95% homology and share a common functional activity are defined herein as sufficiently identical.

As used interchangeably herein, a “CDHN activity”, “biological activity of CDHN” or “CDHN-mediated activity”, includes an activity exerted by a CDHN protein, polypeptide or nucleic acid molecule on a CDHN responsive cell or tissue, or on a CDHN protein substrate, as determined in vivo, or in vitro, according to standard techniques. In one embodiment, a CDHN activity is a direct activity, such as an association with a CDHN target molecule. As used herein, a “target molecule” or “binding partner” is a molecule with which a CDHN protein binds or interacts in nature, such that CDHN mediated function is achieved. A CDHN target molecule can be a non-CDHN molecule or a CDHN protein or polypeptide of the present invention. In one exemplary embodiment, a CDHN target molecule is a CDHN protein. In another exemplary embodiment, a CDHN target molecule is a CDHN substrate (e.g., a cytoplasmic protein, e.g., a protein containing at least one armadillo repeat). Alternatively, a CDHN activity is an indirect activity, such as a cellular signaling or adhesion activity mediated by interaction of the CDHN protein with a CDHN ligand or substrate. The biological activities of CDHN are described herein. For example, the CDHN proteins of the present invention can have one or more of the following activities: 1) modulation of cell adhesion, e.g., cell-cell and cell-substrate adhesion; 2) modulation of cell growth, proliferation, and/or differentiation; 3) modulation of cell motility, e.g., cell migration and cell invasion; 4) modulation of cytoskeletal organization; 5) modulation and maintenance of multicellular organization, e.g., cell sorting, cell polarization, tissue morphogenesis, tissue integrity; 6) modulation of intra- and/or inter-cellular signaling; and 7) modulation of transcriptional regulation of gene expression.

Accordingly, another embodiment of the invention features isolated CDHN proteins and polypeptides having a CDHN activity. Other preferred proteins are CDHN proteins having one or more of the following domains: a cadherin domain, a CA domain, a cadherins extracellular repeated domain signature pattern, a transmembrane domain, or a signal peptide and, preferably, a CDHN activity.

Additional preferred proteins have at least one or more of the following domains: a cadherin domain, a CA domain, a cadherins extracellular repeated domain signature pattern, a transmembrane domain, or a signal peptide, and are, preferably, encoded by a nucleic acid molecule having a nucleotide sequence which hybridizes under stringent hybridization conditions to a nucleic acid molecule comprising the nucleotide sequence of SEQ ID NO:1, 3, 4 or 6.

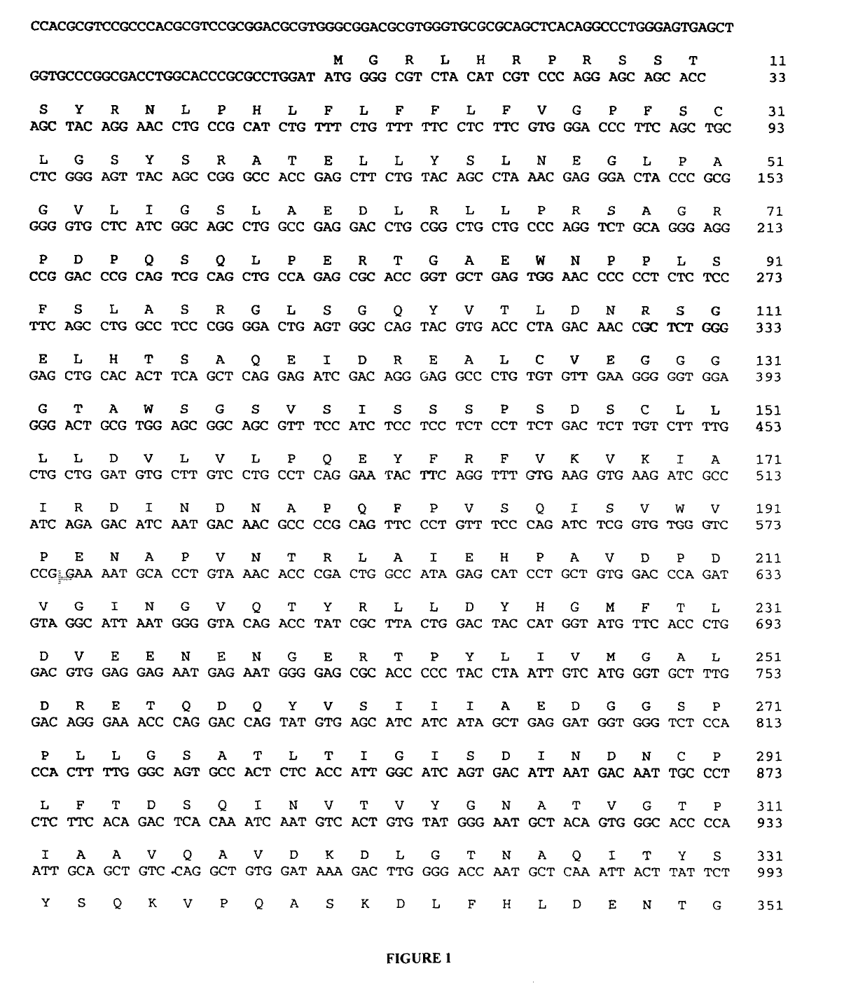

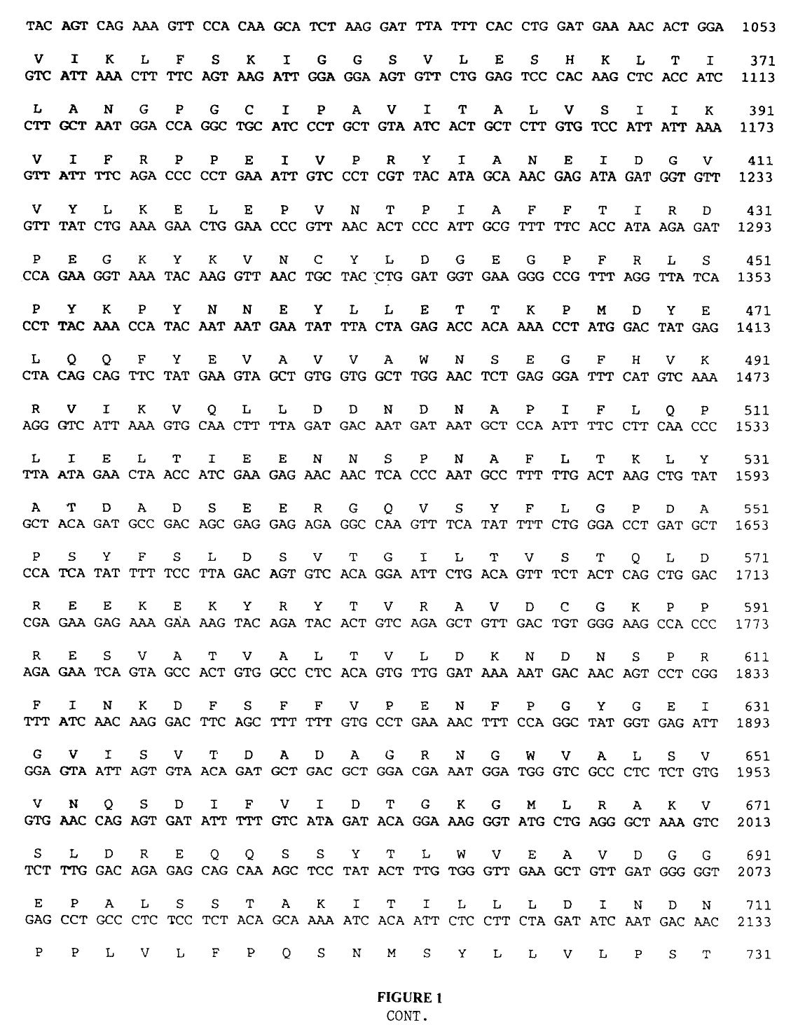

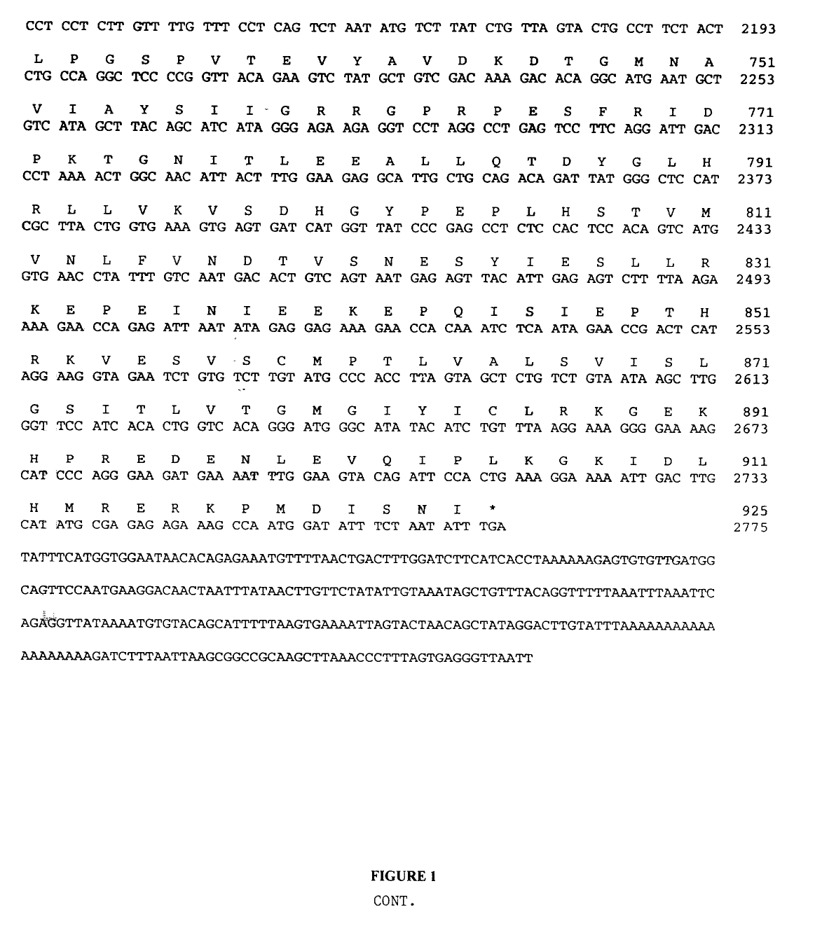

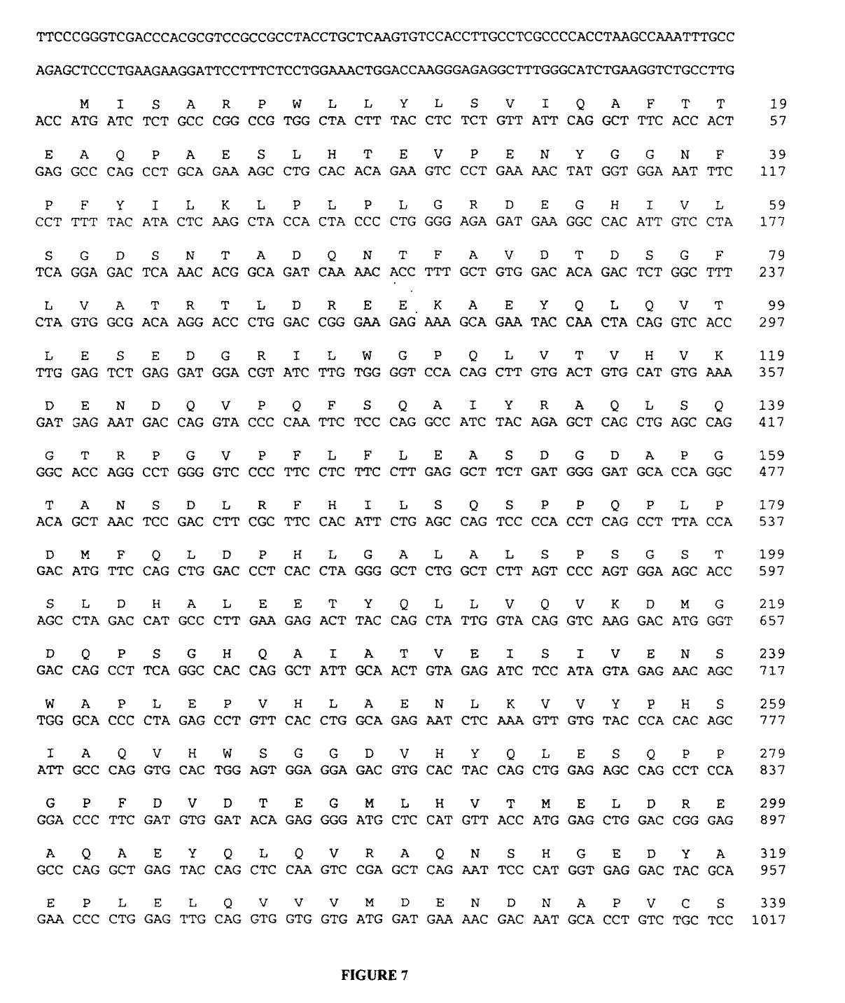

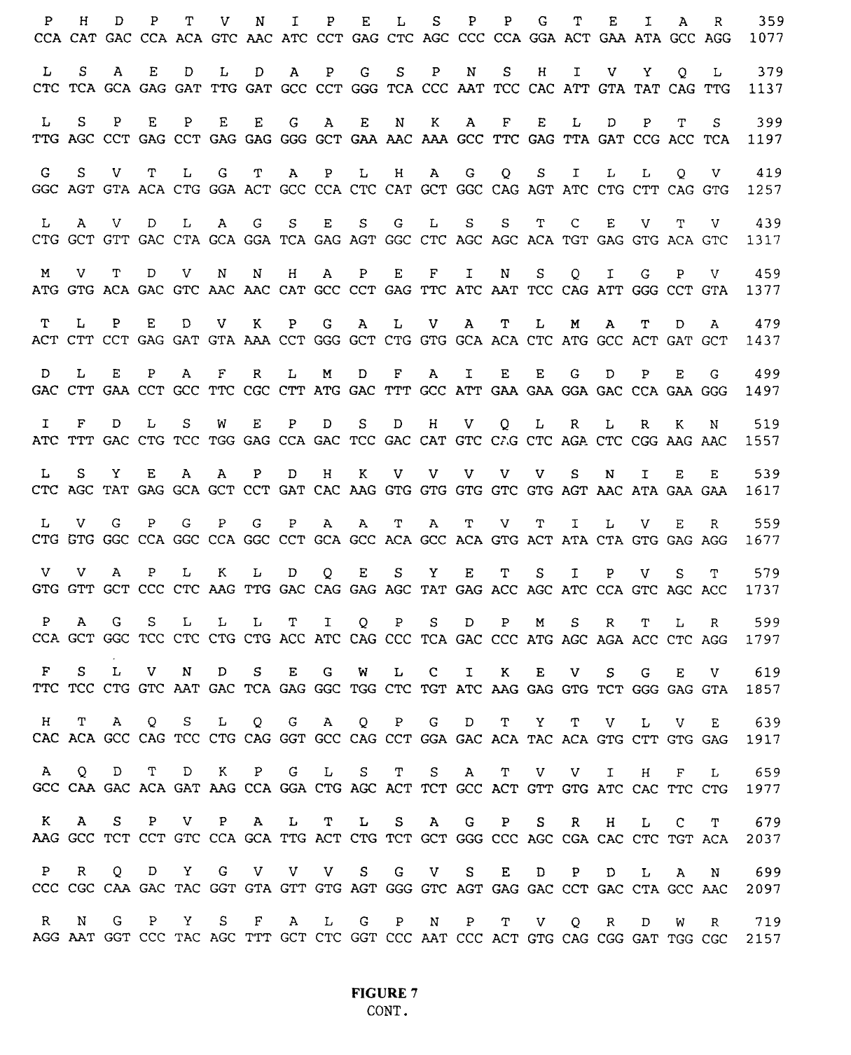

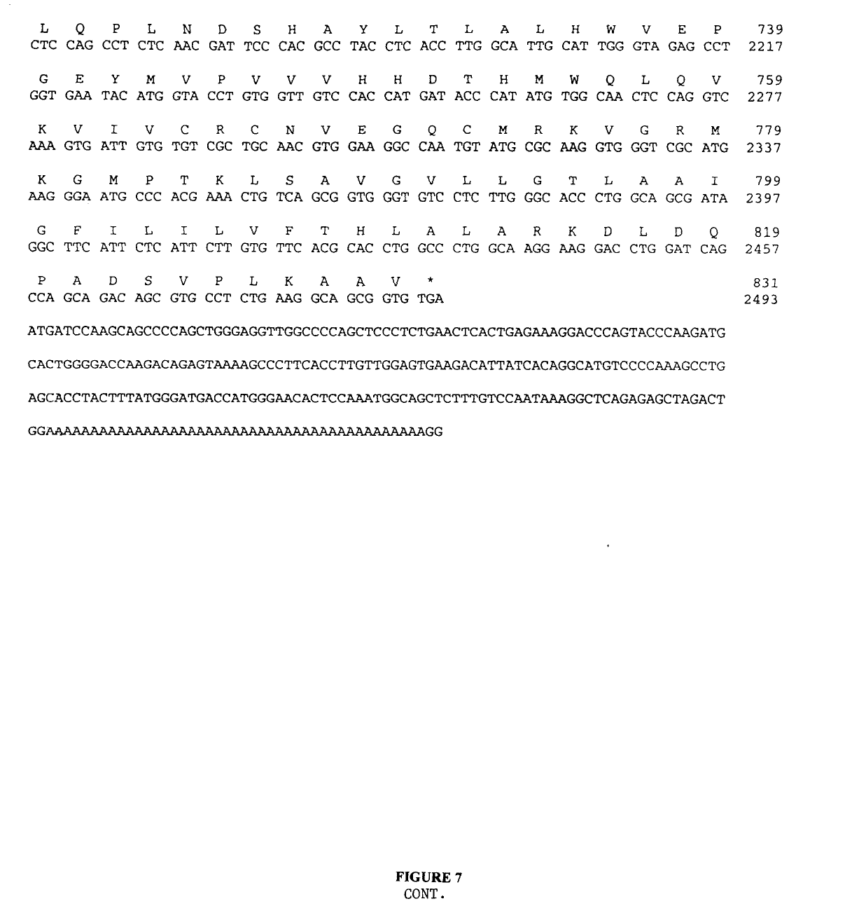

The nucleotide sequence of the isolated human CDHN-1 cDNA and the predicted amino acid sequence of the human CDHN-1 polypeptide are shown in FIG. 1 and in SEQ ID NOs:1 and 2, respectively. The nucleotide sequence of the isolated human CDHN-2 cDNA and the predicted amino acid sequence of the human CDHN-2 polypeptide are shown in FIG. 7 and in SEQ ID NOs:4 and 5, respectively. Plasmids containing the nucleotide sequence encoding human CDHN-1 and CDHN-2 were deposited with the American Type Culture Collection (ATCC), 10801 University Boulevard, Manassas, Va. 20110-2209, on ______ and assigned Accession Numbers ______. These deposits will be maintained under the terms of the Budapest Treaty on the International Recognition of the Deposit of Microorganisms for the Purposes of Patent Procedure. These deposits were made merely as a convenience for those of skill in the art and are not an admission that deposits are required under 35 U.S.C. §112.

The human CDHN-1 gene, which is approximately 3181 nucleotides in length, encodes a protein having a molecular weight of approximately 102 kD and which is approximately 924 amino acid residues in length.

The human CDHN-2 gene, which is approximately 2938 nucleotides in length, encodes a protein having a molecular weight of approximately 91 kD and which is approximately 830 amino acid residues in length.

Various aspects of the invention are described in further detail in the following subsections:

### I. Isolated Nucleic Acid Molecules

One aspect of the invention pertains to isolated nucleic acid molecules that encode CDHN proteins or biologically active portions thereof, as well as nucleic acid fragments sufficient for use as hybridization probes to identify CDHN-encoding nucleic acid molecules (e.g., CDHN mRNA) and fragments for use as PCR primers for the amplification or mutation of CDHN nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated CDHN nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, 3, 4 or 6, or the nucleotide sequence of the DNA insert of the plasmid deposited with ATCC as Accession Number ______, or a portion thereof, can be isolated using standard molecular biology techniques and the sequence information provided herein. Using all or portion of the nucleic acid sequence of SEQ ID NO:1, 3, 4 or 6, or the nucleotide sequence of the DNA insert of the plasmid deposited with ATCC as Accession Number ______ as a hybridization probe, CDHN nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, 3, 4 or 6, or the nucleotide sequence of the DNA insert of the plasmid deposited with ATCC as Accession Number ______ can be isolated by the polymerase chain reaction (PCR) using synthetic oligonucleotide primers designed based upon the sequence of SEQ ID NO:1, 3, 4 or 6, or the nucleotide sequence of the DNA insert of the plasmid deposited with ATCC as Accession Number ______.

A nucleic acid of the invention can be amplified using cDNA, mRNA or, alternatively, genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to CDHN nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO:1, 3, 4 or 6. This cDNA may comprise sequences encoding the human CDHN-1 protein (i.e., “the coding region”, from nucleotides 112-2886), as well as 5′ untranslated sequences (nucleotides 1-111) and 3′ untranslated sequences (nucleotides 2887-3181) of SEQ ID NO:1. Alternatively, the nucleic acid molecule can comprise only the coding region of SEQ ID NO:1 (e.g., nucleotides 112-2886, corresponding to SEQ ID NO:3). This cDNA may comprise sequences encoding the human CDHN-2 protein (i.e., “the coding region”, from nucleotides 162-2654), as well as 5′ untranslated sequences (nucleotides 1-161) and 3′ untranslated sequences (nucleotides 2655-2938) of SEQ ID NO:4. Alternatively, the nucleic acid molecule can comprise only the coding region of SEQ ID NO:4 (e.g., nucleotides 162-2654, corresponding to SEQ ID NO:6).

In another preferred embodiment, an isolated nucleic acid molecule of the invention comprises a nucleic acid molecule which is a complement of the nucleotide sequence shown in SEQ ID NO:1, 3, 4 or 6, or the nucleotide sequence of the DNA insert of the plasmid deposited with ATCC as Accession Number ______, or a portion of any of these nucleotide sequences. A nucleic acid molecule which is complementary to the nucleotide sequence shown in SEQ ID NO:1, 3, 4 or 6, or the nucleotide sequence of the DNA insert of the plasmid deposited with ATCC as Accession Number ______, is one which is sufficiently complementary to the nucleotide sequence shown in SEQ ID NO:1, 3, 4 or 6, or the nucleotide sequence of the DNA insert of the plasmid deposited with ATCC as Accession Number ______ such that it can hybridize to the nucleotide sequence shown in SEQ ID NO:1, 3, 4 or 6, or the nucleotide sequence of the DNA insert of the plasmid deposited with ATCC as Accession Number ______, respectively, thereby forming a stable duplex.

In still another preferred embodiment, an isolated nucleic acid molecule of the present invention comprises a nucleotide sequence which is at least about 50%, 55%, 60%, 65%, 70%, 75%, 80%, 85%, 90%, 95%, 96%, 97%, 98%, 99% or more identical to the entire length of the nucleotide sequence shown in SEQ ID NO:1, 3, 4 or 6, or the entire length of the nucleotide sequence of the DNA insert of the plasmid deposited with ATCC as Accession Number ______, or a portion of any of these nucleotide sequences.

Moreover, the nucleic acid molecule of the invention can comprise only a portion of the nucleic acid sequence of SEQ ID NO:1, 3, 4 or 6, or the nucleotide sequence of the DNA insert of the plasmid deposited with ATCC as Accession Number ______, for example, a fragment which can be used as a probe or primer or a fragment encoding a portion of a CDHN protein, e.g., a biologically active portion of a CDHN protein. The nucleotide sequences determined from the cloning of the CDHN-1 and CDHN-2 genes allow for the generation of probes and primers designed for use in identifying and/or cloning other CDHN family members, as well as CDHN homologues from other species. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1, 3, 4 or 6, or the nucleotide sequence of the DNA insert of the plasmid deposited with ATCC as Accession Number ______ of an anti-sense sequence of SEQ ID NO:1, 3, 4 or 6, or the nucleotide sequence of the DNA insert of the plasmid deposited with ATCC as Accession Number ______, or of a naturally occurring allelic variant or mutant of SEQ ID NO:1, 3, 4 or 6, or the nucleotide sequence of the DNA insert of the plasmid deposited with ATCC as Accession Number ______. In one embodiment, a nucleic acid molecule of the present invention comprises a nucleotide sequence which is greater than 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000-1100, 1100-1200, 1200-1300, 1300-1400, 1400-1500, 1500-1600, 1600-1800, 1800-2000, 2000-2200, 2200-2400, 2400-2600, 2600-2800, 2800-3000, 3000 or more nucleotides in length and hybridizes under stringent hybridization conditions to a nucleic acid molecule of SEQ ID NO:1, 3, 4 or 6, or the nucleotide sequence of the DNA insert of the plasmid deposited with ATCC as Accession Number ______.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4, and 6. Additional stringent conditions can be found in Molecular Cloning: A Laboratory Manual, Sambrook et al., Cold Spring Harbor Press, Cold Spring Harbor, N.Y. (1989), chapters 7, 9, and 11. A preferred, non-limiting example of stringent hybridization conditions includes hybridization in 4×sodium chloride/sodium citrate (SSC), at about 65-70° C. (or alternatively hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or alternatively hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15MNaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C. (see e.g., Church and Gilbert (1984) Proc. Natl. Acad. Sci. USA 81:1991-1995), or alternatively 0.2×SSC, 1% SDS.

Probes based on the CDHN nucleotide sequences can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a CDHN protein, such as by measuring a level of a CDHN-encoding nucleic acid in a sample of cells from a subject e.g., detecting CDHN mRNA levels or determining whether a genomic CDHN gene has been mutated or deleted.

A nucleic acid fragment encoding a “biologically active portion of a CDHN protein” can be prepared by isolating a portion of the nucleotide sequence of SEQ ID NO:1, 3, 4 or 6, or the nucleotide sequence of the DNA insert of the plasmid deposited with ATCC as Accession Number ______ which encodes a polypeptide having a CDHN biological activity (the biological activities of the CDHN proteins are described herein), expressing the encoded portion of the CDHN protein (e.g., by recombinant expression in vitro) and assessing the activity of the encoded portion of the CDHN protein.

The invention further encompasses nucleic acid molecules that differ from the nucleotide sequence shown in SEQ ID NO:1, 3, 4 or 6, or the nucleotide sequence of the DNA insert of the plasmid deposited with ATCC as Accession Number ______ due to degeneracy of the genetic code and thus encode the same CDHN proteins as those encoded by the nucleotide sequence shown in SEQ ID NO:1, 3, 4 or 6, or the nucleotide sequence of the DNA insert of the plasmid deposited with ATCC as Accession Number ______. In another embodiment, an isolated nucleic acid molecule of the invention has a nucleotide sequence encoding a protein having an amino acid sequence shown in SEQ ID NO:2 or 5.

In addition to the CDHN nucleotide sequences shown in SEQ ID NO:1, 3, 4 or 6, or the nucleotide sequence of the DNA insert of the plasmid deposited with ATCC as Accession Number ______, it will be appreciated by those skilled in the art that DNA sequence polymorphisms that lead to changes in the amino acid sequences of the CDHN proteins may exist within a population (e.g., the human population). Such genetic polymorphism in the CDHN genes may exist among individuals within a population due to natural allelic variation. As used herein, the terms “gene” and “recombinant gene” refer to nucleic acid molecules which include an open reading frame encoding a CDHN protein, preferably a mammalian CDHN protein, and can further include non-coding regulatory sequences, and introns.

Allelic variants of human CDHN proteins include both functional and non-functional CDHN proteins. Functional allelic variants are naturally occurring amino acid sequence variants of the human CDHN protein that maintain the ability to bind a CDHN ligand or substrate and/or modulate cell proliferation, differentiation, adhesion, migration and/or signaling mechanisms. Functional allelic variants will typically contain only conservative substitution of one or more amino acids of SEQ ID NO:2 or 5, or substitution, deletion or insertion of non-critical residues in non-critical regions of the protein.

Non-functional allelic variants are naturally occurring amino acid sequence variants of the human CDHN protein that do not have the ability to either bind a CDHN ligand or substrate and/or modulate any of the CDHN activities described herein. Non-functional allelic variants will typically contain a non-conservative substitution, a deletion, or insertion or premature truncation of the amino acid sequence of SEQ ID NO:2 or 5, or a substitution, insertion or deletion in critical residues or critical regions of the protein.

The present invention further provides non-human orthologues of the human CDHN-1 and CDHN-2 proteins. Orthologues of the human CDHN protein are proteins that are isolated from non-human organisms and possess the same CDHN ligand or substrate binding and/or modulation of cell proliferation, differentiation, adhesion, migration and/or signaling mechanisms. Orthologues of the human CDHN protein can readily be identified as comprising an amino acid sequence that is substantially identical to SEQ ID NO:2 or 5.

Moreover, nucleic acid molecules encoding other CDHN family members and, thus, which have a nucleotide sequence which differs from the CDHN sequence of SEQ ID NO:1, 3, 4 or 6, or the nucleotide sequence of the DNA insert of the plasmid deposited with ATCC as Accession Number are intended to be within the scope of the invention. For example, another CDHN cDNA can be identified based on the nucleotide sequence of human CDHN. Moreover, nucleic acid molecules encoding CDHN proteins from different species, and which, thus, have a nucleotide sequence which differs from the CDHN sequence of SEQ ID NO:1, 3, 4 or 6, or the nucleotide sequence of the DNA insert of the plasmid deposited with ATCC as Accession Number ______ are intended to be within the scope of the invention. For example, a mouse CDHN cDNA can be identified based on the nucleotide sequence of a human CDHN.

Nucleic acid molecules corresponding to natural allelic variants and homologues of the CDHN cDNAs of the invention can be isolated based on their homology to the CDHN nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the CDHN cDNAs of the invention can further be isolated by mapping to the same chromosome or locus as the CDHN gene.

Accordingly, in another embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:1, 3, 4 or 6, or the nucleotide sequence of the DNA insert of the plasmid deposited with ATCC as Accession Number ______. In other embodiment, the nucleic acid is at least 50-100, 100-150, 150-200, 200-250, 250-300, 300-350, 350-400, 400-450, 450-500, 500-550, 550-600, 600-650, 650-700, 700-750, 750-800, 800-850, 850-900, 900-950,950-1000,1000-1100, 1100-1200, 1200-1300,1300-1400, 1400-1500, 1500-1600, 1600-1800, 1800-2000, 2000-2200, 2200-2400, 2400-2600, 2600-2800, 2800-3000, 3000 or more nucleotides in length. As used herein, the term “hybridizes under stringent conditions” is intended to describe conditions for hybridization and washing under which nucleotide sequences at least 60% identical to each other typically remain hybridized to each other. Preferably, the conditions are such that sequences at least about 70%, more preferably at least about 80%, even more preferably at least about 85% or 90% identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6×sodium chloride/sodium citrate (SSC) at about 45° C., followed by one or more washes in 0.2×SSC, 0.1% SDS at 50° C., preferably at 55° C., more preferably at 60° C., and even more preferably at 65° C. Ranges intermediate to the above-recited values, e.g., at 60-65° C. or at 55-60 ° C. are also intended to be encompassed by the present invention. Preferably, an isolated nucleic acid molecule of the invention that hybridizes under stringent conditions to the sequence of SEQ ID NO:1, 3, 4 or 6 and corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the CDHN sequences that may exist in the population, the skilled artisan will further appreciate that changes can be introduced by mutation into the nucleotide sequences of SEQ ID NO:1, 3, 4 or 6, or the nucleotide sequence of the DNA insert of the plasmid deposited with ATCC as Accession Number ______, thereby leading to changes in the amino acid sequence of the encoded CDHN protein, without altering the functional ability of the CDHN protein. For example, nucleotide substitutions leading to amino acid substitutions at “non-essential” amino acid residues can be made in the sequence of SEQ ID NO:1, 3, 4 or 6, or the nucleotide sequence of the DNA insert of the plasmid deposited with ATCC as Accession Number ______. A “non-essential” amino acid residue is a residue that can be altered from the wild-type sequence of CDHN (e.g., the sequence of SEQ ID NO:2 or 5) without altering the biological activity, whereas an “essential” amino acid residue is required for biological activity. For example, amino acid residues that are conserved among the CDHN proteins of the present invention, e.g., those present in a cadherin domain, a CA domain, or a cadherins extracellular repeated domain signature pattern, are predicted to be particularly unamenable to alteration. Furthermore, additional amino acid residues that are conserved between the CDHN proteins of the present invention and other members of the CDHN family are not likely to be amenable to alteration.

Accordingly, another aspect of the invention pertains to nucleic acid molecules encoding CDHN proteins that contain changes in amino acid residues that are not essential for activity. Such CDHN proteins differ in amino acid sequence from SEQ ID NO:2 or 5, yet retain biological activity. In one embodiment, the isolated nucleic acid molecule comprises a nucleotide sequence encoding a protein, wherein the protein comprises an amino acid sequence at least about 50%, 55%, 60%, 65%, 70%, 75%, 80%, 85%, 90%, 95%, 96% 97%, 98%, 99% or more identical to SEQ ID NO:2 or 5.

An isolated nucleic acid molecule encoding a CDHN protein identical to the protein of SEQ ID NO:2 or 5 can be created by introducing one or more nucleotide substitutions, additions or deletions into the nucleotide sequence of SEQ ID NO:1, 3, 4 or 6, or the nucleotide sequence of the DNA insert of the plasmid deposited with ATCC as Accession Number ______ such that one or more amino acid substitutions, additions or deletions are introduced into the encoded protein. Mutations can be introduced into SEQ ID NO:1, 3, 4 or 6, or the nucleotide sequence of the DNA insert of the plasmid deposited with ATCC as Accession Number ______ by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a CDHN protein is preferably replaced with another amino acid residue from the same side chain family. Alternatively, in another embodiment, mutations can be introduced randomly along all or part of a CDHN coding sequence, such as by saturation mutagenesis, and the resultant mutants can be screened for CDHN biological activity to identify mutants that retain activity. Following mutagenesis of SEQ ID NO:1, 3, 4 or 6, or the nucleotide sequence of the DNA insert of the plasmid deposited with ATCC as Accession Number ______, the encoded protein can be expressed recombinantly and the activity of the protein can be determined.

In a preferred embodiment, a mutant CDHN protein can be assayed for the ability to: 1) modulate of cell adhesion, e.g., cell-cell and cell-substrate adhesion; 2) modulate cell growth, proliferation, and/or differentiation; 3) modulate of cell motility, e.g., cell migration and cell invasion; 4) modulate cytoskeletal organization; 5) modulate and maintain multicellular organization, e.g., cell sorting, cell polarization, tissue morphogenesis, tissue integrity; 6) modulate intra- and/or inter-cellular signaling; and 7) modulate transcriptional regulation of gene expression.

In addition to the nucleic acid molecules encoding CDHN proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire CDHN coding strand, or to only a portion thereof. In one embodiment, an antisense nucleic acid molecule is antisense to a “coding region” of the coding strand of a nucleotide sequence encoding a CDHN. The term “coding region” refers to the region of the nucleotide sequence comprising codons which are translated into amino acid residues (e.g., the coding region of human CDHN-1 corresponds to SEQ ID NO:3, the coding region of human CDHN-2 corresponds to SEQ ID NO:6). In another embodiment, the antisense nucleic acid molecule is antisense to a “noncoding region” of the coding strand of a nucleotide sequence encoding a CDHN. The term “noncoding region” refers to 5′ and 3′ sequences which flank the coding region that are not translated into amino acids (i.e., also referred to as 5′ and 3′ untranslated regions).

Given the coding strand sequences encoding CDHN-1 and CDHN-2 disclosed herein (e.g., SEQ ID NO:3 and 6), antisense nucleic acids of the invention can be designed according to the rules of Watson and Crick base pairing. The antisense nucleic acid molecule can be complementary to the entire coding region of CDHN mRNA, but more preferably is an oligonucleotide which is antisense to only a portion of the coding or noncoding region of CDHN mRNA. For example, the antisense oligonucleotide can be complementary to the region surrounding the translation start site of CDHN mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5- oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a CDHN protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave CDHN mRNA transcripts to thereby inhibit translation of CDHN mRNA. A ribozyme having specificity for a CDHN-encoding nucleic acid can be designed based upon the nucleotide sequence of a CDHN cDNA disclosed herein (i.e., SEQ ID NO:1, 3, 4 or 6, or the nucleotide sequence of the DNA insert of the plasmid deposited with ATCC as Accession Number ______). For example, a derivative of a Tetrahymena L-19 IVS RNA can be constructed in which the nucleotide sequence of the active site is complementary to the nucleotide sequence to be cleaved in a CDHN-encoding mRNA. See, e.g., Cech et al. U.S. Pat. No. 4,987,071; and Cech et al. U.S. Pat. No. 5,116,742. Alternatively, CDHN mRNA can be used to select a catalytic RNA having a specific ribonuclease activity from a pool of RNA molecules. See, e.g., Bartel, D. and Szostak, J. W. (1993) Science 261:1411-1418.

Alternatively, CDHN gene expression can be inhibited by targeting nucleotide sequences complementary to the regulatory region of the CDHN (e.g., the CDHN promoter and/or enhancers; e.g., nucleotides 1-111 of SEQ ID NO:1 or nucleotides 1-161 of SEQ ID NO:4) to form triple helical structures that prevent transcription of the CDHN gene in target cells. See generally, Helene, C. (1991) Anticancer Drug Des. 6(6): 569-84; Helene, C. et al. (1992) Ann. N.Y Acad. Sci. 660:27-36; and Maher, L. J. (1992) Bioassays 14(12):807-15.

In yet another embodiment, the CDHN nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of CDHN nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of CDHN nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of CDHN can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of CDHN nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Alternatively, the expression characteristics of an endogenous CDHN gene within a cell line or microorganism may be modified by inserting a heterologous DNA regulatory element into the genome of a stable cell line or cloned microorganism such that the inserted regulatory element is operatively linked with the endogenous CDHN gene. For example, an endogenous CDHN gene which is normally “transcriptionally silent”, i e., a CDHN gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous CDHN gene may be activated by insertion of a promiscuous regulatory element that works across cell types.

A heterologous regulatory element may be inserted into a stable cell line or cloned microorganism, such that it is operatively linked with an endogenous CDHN gene, using techniques, such as targeted homologous recombination, which are well known to those of skill in the art, and described, e.g., in Chappel, U.S. Pat. No. 5,272,071; PCT publication No. WO 91/06667, published May 16, 1991.

### II. Isolated CDHN Proteins and Anti-CDHN Antibodies

One aspect of the invention pertains to isolated CDHN proteins, and biologically active portions thereof, as well as polypeptide fragments suitable for use as immunogens to raise anti-CDHN antibodies. In one embodiment, native CDHN proteins can be isolated from cells or tissue sources by an appropriate purification scheme using standard protein purification techniques. In another embodiment, CDHN proteins are produced by recombinant DNA techniques. Alternative to recombinant expression, a CDHN protein or polypeptide can be synthesized chemically using standard peptide synthesis techniques.

An “isolated” or “purified” protein or biologically active portion thereof is substantially free of cellular material or other contaminating proteins from the cell or tissue source from which the CDHN protein is derived, or substantially free from chemical precursors or other chemicals when chemically synthesized. The language “substantially free of cellular material” includes preparations of CDHN protein in which the protein is separated from cellular components of the cells from which it is isolated or recombinantly produced. In one embodiment, the language “substantially free of cellular material” includes preparations of CDHN protein having less than about 30% (by dry weight) of non-CDHN protein (also referred to herein as a “contaminating protein”), more preferably less than about 20% of non-CDHN protein, still more preferably less than about 10% of non-CDHN protein, and most preferably less than about 5% non-CDHN protein. When the CDHN protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of CDHN protein in which the protein is separated from chemical precursors or other chemicals which are involved in the synthesis of the protein. In one embodiment, the language “substantially free of chemical precursors or other chemicals” includes preparations of CDHN protein having less than about 30% (by dry weight) of chemical precursors or non-CDHN chemicals, more preferably less than about 20% chemical precursors or non-CDHN chemicals, still more preferably less than about 10% chemical precursors or non-CDHN chemicals, and most preferably less than about 5% chemical precursors or non-CDHN chemicals.

As used herein, a “biologically active portion” of a CDHN protein includes a fragment of a CDHN protein which participates in an interaction between CDHN molecules, or in an interaction between a CDHN molecule and a non-CDHN molecule. Biologically active portions of a CDHN protein include peptides comprising amino acid sequences sufficiently identical to or derived from the amino acid sequence of the CDHN protein, e.g., the amino acid sequence shown in SEQ ID NO:2 or 5, which include less amino acids than the full length CDHN protein, and exhibit at least one activity of a CDHN protein. Typically, biologically active portions comprise a domain or motif with at least one activity of the CDHN protein, e.g., modulation of cell proliferation, differentiation, adhesion, migration and/or signaling mechanisms. A biologically active portion of a CDHN protein can be a polypeptide which is, for example, 25, 50, 75, 100, 125, 150, 175, 200, 250, 300, 400, 500 or more amino acids in length. Biologically active portions of a CDHN protein can be used as targets for developing agents which modulate a CDHN mediated activity, e.g., cell proliferation, differentiation, adhesion, migration and/or signaling mechanisms.

In one embodiment, a biologically active portion of a CDHN protein comprises at least one, preferably two, three, four, five or more cadherin domains. In another embodiment, a biologically active portion of a CDHN protein comprises at least one, preferably two, three, four, five or six CA domains. In another embodiment, a biologically active portion of a CDHN protein of the present invention may contain at least one, preferably two, three, four, five or more, cadherin domains, and at least one or more of the following domains: a CA domain, a cadherins extracellular repeated domain signature pattern, a transmembrane domain, or a signal peptide. In a further embodiment, a biologically active portion of a CDHN protein of the present invention may contain at least one, preferably two, three, four, five, or six CA domains, and at least one or more of the following domains: a cadherin domain, a cadherins extracellular repeated domain signature pattern, a transmembrane domain, or a signal peptide. Moreover, other biologically active portions, in which other regions of the protein are deleted, can be prepared by recombinant techniques and evaluated for one or more of the functional activities of a native CDHN protein.

In a preferred embodiment, the CDHN protein has an amino acid sequence shown in SEQ ID NO:2 or 5. In other embodiments, the CDHN protein is substantially identical to SEQ ID NO:2 or 5 and retains the functional activity of the protein of SEQ ID NO:2 or 5, yet differs in amino acid sequence due to natural allelic variation or mutagenesis, as described in detail in subsection I above. Accordingly, in another embodiment, the CDHN protein is a protein which comprises an amino acid sequence at least about 50%, 55%, 60%, 65%, 70%, 75%, 80%, 85%, 90%, 95%, 96%, 97%, 98%, 99% or more identical to SEQ ID NO:2 or 5.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the CDHN amino acid sequence of SEQ ID NO:2 having 924 amino acid residues, at least 277, preferably at least 370, more preferably at least 462, even more preferably at least 555, and even more preferably at least 647 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci., 4: 11-17 (1988)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to CDHN nucleic acid molecules of the invention. BLAST protein searches can be performed with the XBLAST program, score=100, wordlength=3 to obtain amino acid sequences homologous to CDHN protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention also provides CDHN chimeric or fusion proteins. As used herein, a CDHN “chimeric protein” or “fusion protein” comprises a CDHN polypeptide operatively linked to a non-CDHN polypeptide. A “CDHN polypeptide” refers to a polypeptide having an amino acid sequence corresponding to a CDHN (e.g., CDHN-1, CDHN-2) molecule, whereas a “non-CDHN polypeptide” refers to a polypeptide having an amino acid sequence corresponding to a protein which is not substantially homologous to the CDHN protein, e.g., a protein which is different from the CDHN protein and which is derived from the same or a different organism. Within a CDHN fusion protein the CDHN polypeptide can correspond to all or a portion of a CDHN protein. In a preferred embodiment, a CDHN fusion protein comprises at least one biologically active portion of a CDHN protein. In another preferred embodiment, a CDHN fusion protein comprises at least two biologically active portions of a CDHN protein. Within the fusion protein, the term “operatively linked” is intended to indicate that the CDHN polypeptide and the non-CDHN polypeptide are fused in-frame to each other. The non-CDHN polypeptide can be fused to the N-terminus or C-terminus of the CDHN polypeptide.

For example, in one embodiment, the fusion protein is a GST-CDHN fusion protein in which the CDHN sequences are fused to the C-terminus of the GST sequences. Such fusion proteins can facilitate the purification of recombinant CDHN.

In another embodiment, the fusion protein is a CDHN protein containing a heterologous signal sequence at its N-terminus. In certain host cells (e.g., mammalian host cells), expression and/or secretion of CDHN can be increased through use of a heterologous signal sequence.

The CDHN fusion proteins of the invention can be incorporated into pharmaceutical compositions and administered to a subject in vivo. The CDHN fusion proteins can be used to affect the bioavailability of a CDHN ligand or substrate. Use of CDHN fusion proteins may be useful therapeutically for the treatment of disorders caused by, for example, (i) aberrant modification or mutation of a gene encoding a CDHN protein; (ii) mis-regulation of a CDHN gene; and (iii) aberrant post-translational modification of a CDHN protein.

Moreover, the CDHN fusion proteins of the invention can be used as immunogens to produce anti-CDHN antibodies in a subject, to purify CDHN ligands and in screening assays to identify molecules which inhibit the interaction of CDHN with a CDHN substrate.

Preferably, a CDHN chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A CDHN-encoding nucleic acid can be cloned into such an expression vector such that the fusion moiety is linked in-frame to the CDHN protein.

The present invention also pertains to variants of the CDHN proteins which function as either CDHN agonists (mimetics) or as CDHN antagonists. Variants of the CDHN proteins can be generated by mutagenesis, e.g., discrete point mutation or truncation of a CDHN protein. An agonist of the CDHN proteins can retain substantially the same, or a subset, of the biological activities of the naturally occurring form of a CDHN protein. An antagonist of a CDHN protein can inhibit one or more of the activities of the naturally occurring form of the CDHN protein by, for example, competitively modulating a CDHN-mediated activity of a CDHN protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the CDHN protein.

In one embodiment, variants of a CDHN protein which function as either CDHN agonists (mimetics) or as CDHN antagonists can be identified by screening combinatorial libraries of mutants, e.g., truncation mutants, of a CDHN protein for CDHN protein agonist or antagonist activity. In one embodiment, a variegated library of CDHN variants is generated by combinatorial mutagenesis at the nucleic acid level and is encoded by a variegated gene library. A variegated library of CDHN variants can be produced by, for example, enzymatically ligating a mixture of synthetic oligonucleotides into gene sequences such that a degenerate set of potential CDHN sequences is expressible as individual polypeptides, or alternatively, as a set of larger fusion proteins (e.g., for phage display) containing the set of CDHN sequences therein. There are a variety of methods which can be used to produce libraries of potential CDHN variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential CDHN sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a CDHN protein coding sequence can be used to generate a variegated population of CDHN fragments for screening and subsequent selection of variants of a CDHN protein. In one embodiment, a library of coding sequence fragments can be generated by treating a double stranded PCR fragment of a CDHN coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with SI nuclease, and ligating the resulting fragment library into an expression vector. By this method, an expression library can be derived which encodes N-terminal, C-terminal and internal fragments of various sizes of the CDHN protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of CDHN proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify CDHN variants (Arkin and Yourvan (1992) Proc. Natl. Acad. Sci. USA 89:7811-7815; Delgrave et al. (1993) Protein Engineering 6(3): 327-331).

In one embodiment, cell based assays can be exploited to analyze a variegated CDHN library. For example, a library of expression vectors can be transfected into a cell line, e.g., a mammalian cell line, which ordinarily responds to a CDHN ligand in a particular CDHN ligand-dependent manner. The transfected cells are then contacted with a CDHN ligand and the effect of expression of the mutant on, e.g., modulation of cell proliferation, differentiation, adhesion, migration and/or signaling mechanisms can be detected. Plasmid DNA can then be recovered from the cells which score for inhibition, or alternatively, potentiation of signaling by the CDHN ligand, and the individual clones further characterized.

An isolated CDHN protein, or a portion or fragment thereof, can be used as an immunogen to generate antibodies that bind CDHN using standard techniques for polyclonal and monoclonal antibody preparation. A full-length CDHN protein can be used or, alternatively, the invention provides antigenic peptide fragments of CDHN for use as immunogens. The antigenic peptide of CDHN comprises at least 8 amino acid residues of the amino acid sequence shown in SEQ ID NO:2 or 5 and encompasses an epitope of CDHN such that an antibody raised against the peptide forms a specific immune complex with the CDHN protein. Preferably, the antigenic peptide comprises at least 10 amino acid residues, more preferably at least 15 amino acid residues, even more preferably at least 20 amino acid residues, and most preferably at least 30 amino acid residues.

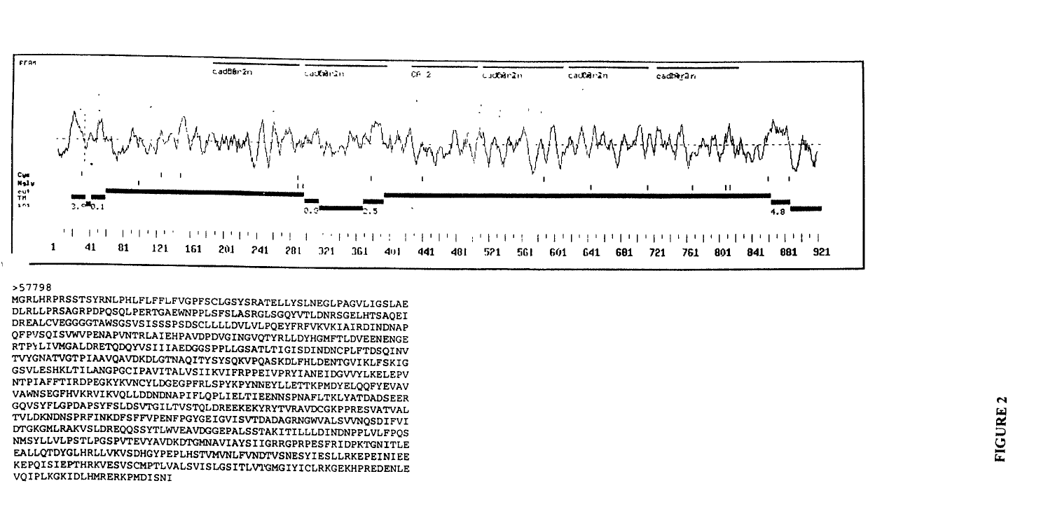

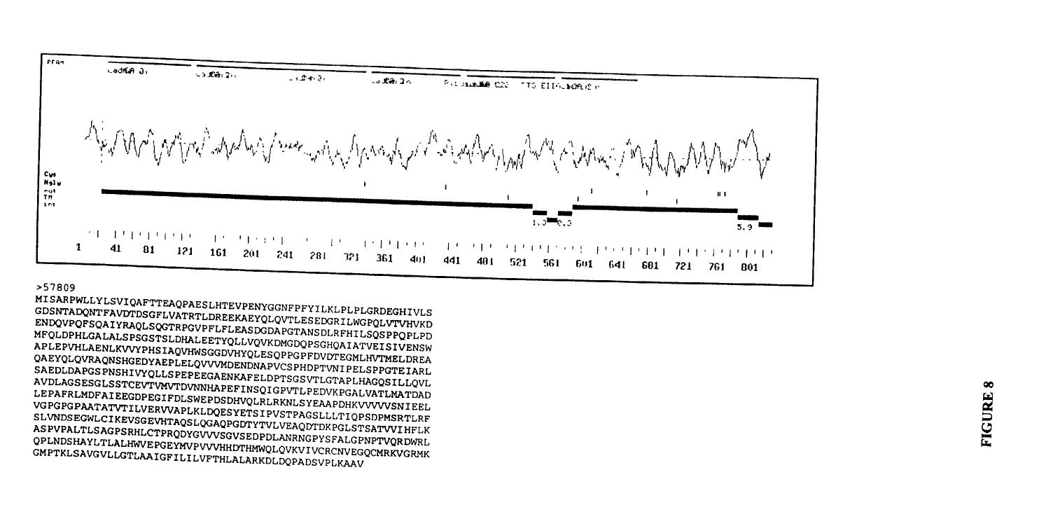

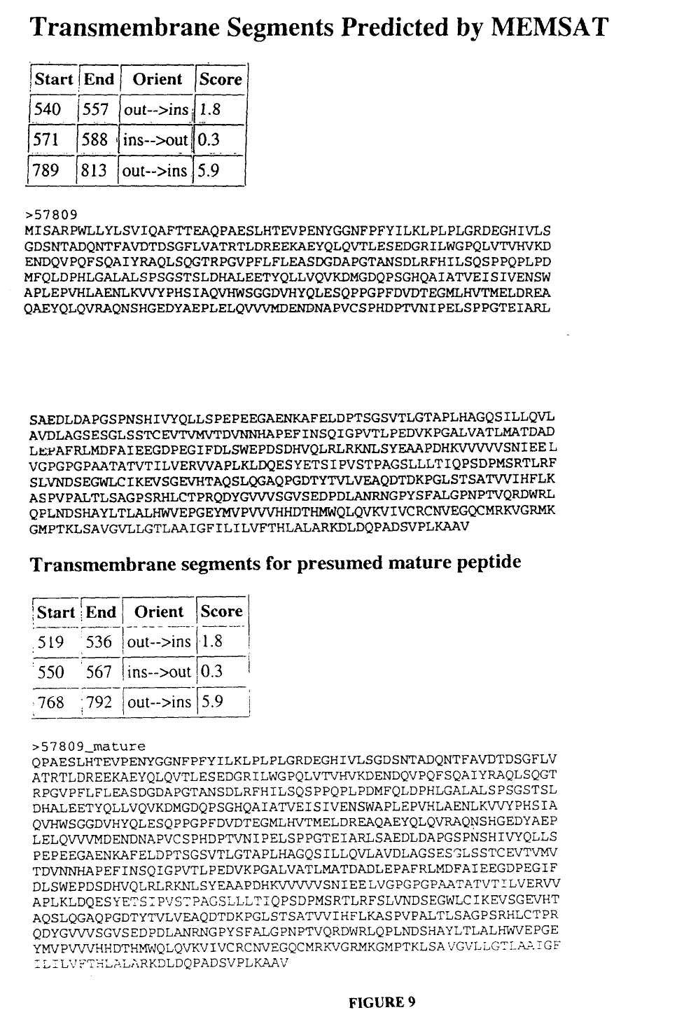

Preferred epitopes encompassed by the antigenic peptide are regions of CDHN that are located on the surface of the protein, e.g., hydrophilic regions, as well as regions with high antigenicity (see, for example, FIGS. 2 and 8).

A CDHN immunogen typically is used to prepare antibodies by immunizing a suitable subject, (e.g., rabbit, goat, mouse or other mammal) with the immunogen. An appropriate immunogenic preparation can contain, for example, recombinantly expressed CDHN protein or a chemically synthesized CDHN polypeptide. The preparation can further include an adjuvant, such as Freund's complete or incomplete adjuvant, or similar immunostimulatory agent. Immunization of a suitable subject with an immunogenic CDHN preparation induces a polyclonal anti-CDHN antibody response.

Accordingly, another aspect of the invention pertains to anti-CDHN antibodies. The term “antibody” as used herein refers to immunoglobulin molecules and immunologically active portions of immunoglobulin molecules, i.e., molecules that contain an antigen binding site which specifically binds (immunoreacts with) an antigen, such as a CDHN. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind CDHN molecules. The term “monoclonal antibody” or “monoclonal antibody composition”, as used herein, refers to a population of antibody molecules that contain only one species of an antigen binding site capable of immunoreacting with a particular epitope of CDHN. A monoclonal antibody composition thus typically displays a single binding affinity for a particular CDHN protein with which it immunoreacts.

Polyclonal anti-CDHN antibodies can be prepared as described above by immunizing a suitable subject with a CDHN immunogen. The anti-CDHN antibody titer in the immunized subject can be monitored over time by standard techniques, such as with an enzyme linked immunosorbent assay (ELISA) using immobilized CDHN. If desired, the antibody molecules directed against CDHN can be isolated from the mammal (e.g., from the blood) and further purified by well known techniques, such as protein A chromatography to obtain the IgG fraction. At an appropriate time after immunization, e.g., when the anti-CDHN antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem .255:4980-83; Yeh et al. (1976) Proc. Natl. Acad. Sci USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lemer (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a CDHN immunogen as described above, and the culture supernatants of the resulting hybridoma cells are screened to identify a hybridoma producing a monoclonal antibody that binds CDHN.

Any of the many well known protocols used for fusing lymphocytes and immortalized cell lines can be applied for the purpose of generating an anti-CDHN monoclonal antibody (see, e.g., G. Galfre et al. (1977) Nature 266:55052; Gefter et al. Somatic Cell Genet., cited supra; Lemer, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind CDHN, e.g., using a standard ELISA assay.

Alternative to preparing monoclonal antibody-secreting hybridomas, a monoclonal anti-CDHN antibody can be identified and isolated by screening a recombinant combinatorial immunoglobulin library (e.g., an antibody phage display library) with CDHN to thereby isolate immunoglobulin library members that bind CDHN. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-CDHN antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-CDHN antibody (e.g., monoclonal antibody) can be used to isolate CDHN by standard techniques, such as affinity chromatography or immunoprecipitation. An anti-CDHN antibody can facilitate the purification of natural CDHN from cells and of recombinantly produced CDHN expressed in host cells. Moreover, an anti-CDHN antibody can be used to detect CDHN protein (e.g., in a cellular lysate or cell supernatant) in order to evaluate the abundance and pattern of expression of the CDHN protein. Anti-CDHN antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### III. Recombinant Expression Vectors and Host Cells

Another aspect of the invention pertains to vectors, preferably expression vectors, containing a nucleic acid encoding a CDHN protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., CDHN proteins, mutant forms of CDHN proteins, fusion proteins, and the like).

The recombinant expression vectors of the invention can be designed for expression of CDHN proteins in prokaryotic or eukaryotic cells. For example, CDHN proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in CDHN activity assays, (e.g., direct assays or competitive assays described in detail below), or to generate antibodies specific for CDHN proteins, for example. In a preferred embodiment, a CDHN fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11 d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the CDHN expression vector is a yeast expression vector. Examples of vectors for expression in yeast S. cerevisiae include pYepSec1 (Baldari, et al., (1987) Embo J. 6:229-234), pMFa (Kurjan and Herskowitz, (1982) Cell 30:933-943), pJRY88 (Schultz et al, (1987) Gene 54:113-123), pYES2 (Invitrogen Corporation, San Diego, Calif.), and picZ (InVitrogen Corp, San Diego, Calif.).

Alternatively, CDHN proteins can be expressed in insect cells using baculovirus expression vectors. Baculovirus vectors available for expression of proteins in cultured insect cells (e.g., Sf 9 cells) include the pAc series (Smith et al. (1 983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to CDHN mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a CDHN nucleic acid molecule of the invention is introduced, e.g., a CDHN nucleic acid molecule within a recombinant expression vector or a CDHN nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a CDHN protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a CDHN protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a CDHN protein. Accordingly, the invention further provides methods for producing a CDHN protein using the host cells of the invention. In one embodiment, the method comprises culturing the host cell of the invention (into which a recombinant expression vector encoding a CDHN protein has been introduced) in a suitable medium such that a CDHN protein is produced. In another embodiment, the method further comprises isolating a CDHN protein from the medium or the host cell.

The host cells of the invention can also be used to produce non-human transgenic animals. For example, in one embodiment, a host cell of the invention is a fertilized oocyte or an embryonic stem cell into which CDHN coding sequences have been introduced. Such host cells can then be used to create non-human transgenic animals in which exogenous CDHN sequences have been introduced into their genome or homologous recombinant animals in which endogenous CDHN sequences have been altered. Such animals are useful for studying the function and/or activity of a CDHN and for identifying and/or evaluating modulators of CDHN activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous CDHN gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a CDHN-encoding nucleic acid into the male pronuclei of a fertilized oocyte, e.g., by microinjection, retroviral infection, and allowing the oocyte to develop in a pseudopregnant female foster animal. The CDHN cDNA sequence of SEQ ID NO:1 or 4 can be introduced as a transgene into the genome of a non-human animal. Alternatively, a nonhuman homologue of a human CDHN gene, such as a mouse or rat CDHN gene, can be used as a transgene. Alternatively, a CDHN gene homologue, such as another CDHN family member, can be isolated based on hybridization to the CDHN cDNA sequences of SEQ ID NO:1, 3 4 or 6, or the DNA insert of the plasmid deposited with ATCC as Accession Number ______ (described further in subsection I above) and used as a transgene. Intronic sequences and polyadenylation signals can also be included in the transgene to increase the efficiency of expression of the transgene. A tissue-specific regulatory sequence(s) can be operably linked to a CDHN transgene to direct expression of a CDHN protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a CDHN transgene in its genome and/or expression of CDHN mRNA in tissues or cells of the animals. A transgenic founder animal can then be used to breed additional animals carrying the transgene. Moreover, transgenic animals carrying a transgene encoding a CDHN protein can further be bred to other transgenic animals carrying other transgenes.

To create a homologous recombinant animal, a vector is prepared which contains at least a portion of a CDHN gene into which a deletion, addition or substitution has been introduced to thereby alter, e.g., functionally disrupt, the CDHN gene. The CDHN gene can be a human gene (e.g., the cDNA of SEQ ID NO:3 or 6), but more preferably, is a non-human homologue of a human CDHN gene (e.g., a cDNA isolated by stringent hybridization with the nucleotide sequence of SEQ ID NO:1 or 4). For example, a mouse CDHN gene can be used to construct a homologous recombination nucleic acid molecule, e.g., a vector, suitable for altering an endogenous CDHN gene in the mouse genome. In a preferred embodiment, the homologous recombination nucleic acid molecule is designed such that, upon homologous recombination, the endogenous CDHN gene is functionally disrupted (i.e., no longer encodes a functional protein; also referred to as a “knock out” vector). Alternatively, the homologous recombination nucleic acid molecule can be designed such that, upon homologous recombination, the endogenous CDHN gene is mutated or otherwise altered but still encodes functional protein (e.g., the upstream regulatory region can be altered to thereby alter the expression of the endogenous CDHN protein). In the homologous recombination nucleic acid molecule, the altered portion of the CDHN gene is flanked at its 5′ and 3′ ends by additional nucleic acid sequence of the CDHN gene to allow for homologous recombination to occur between the exogenous CDHN gene carried by the homologous recombination nucleic acid molecule and an endogenous CDHN gene in a cell, e.g., an embryonic stem cell. The additional flanking CDHN nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced CDHN gene has homologously recombined with the endogenous CDHN gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

### IV. Pharmaceutical Compositions

The CDHN nucleic acid molecules, fragments of CDHN proteins, and anti-CDHN antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a CDHN protein or an anti-CDHN antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### V. Uses and Methods of the Invention

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, a CDHN protein of the invention has one or more of the following activities: 1) modulation of cell adhesion, e.g., cell-cell and cell-substrate adhesion; 2) modulation of cell growth, proliferation, and/or differentiation; 3) modulation of cell motility, e.g., cell migration and cell invasion; 4) modulation of cytoskeletal organization; 5) modulation and maintenance of multicellular organization, e.g., cell sorting, cell polarization, tissue morphogenesis, tissue integrity; 6) modulation of intra- and/or inter-cellular signaling; and 7) modulation of transcriptional regulation of gene expression. The isolated nucleic acid molecules of the invention can be used, for example, to express CDHN protein (e.g., via a recombinant expression vector in a host cell in gene therapy applications), to detect CDHN mRNA (e.g., in a biological sample) or a genetic alteration in a CDHN gene, and to modulate CDHN activity, as described further below. The CDHN proteins can be used to treat disorders characterized by insufficient or excessive production of a CDHN substrate or production of CDHN inhibitors. In addition, the CDHN proteins can be used to screen for naturally occurring CDHN substrates, to screen for drugs or compounds which modulate CDHN activity, as well as to treat disorders characterized by insufficient or excessive production of CDHN protein or production of CDHN protein forms which have decreased, aberrant or unwanted activity compared to CDHN wild type protein (e.g., cadherin-associated disorders, such as central nervous system (CNS) disorders, cardiovascular disorders, musculoskeletal disorders, gastrointestinal disorders, inflammatory or immune system disorders, or cell proliferation, growth, differentiation, adhesion, or migration disorders).

Moreover, the anti-CDHN antibodies of the invention can be used to detect and isolate CDHN proteins, regulate the bioavailability of CDHN proteins, and modulate CDHN activity.

### A. Screening Assays

The invention provides a method (also referred to herein as a “screening assay”) for identifying modulators, i.e., candidate or test compounds or agents (e.g., peptides, peptidomimetics, small molecules or other drugs) which bind to CDHN proteins, have a stimulatory or inhibitory effect on, for example, CDHN expression or CDHN activity, or have a stimulatory or inhibitory effect on, for example, the expression or activity of a CDHN substrate.

In one embodiment, the invention provides assays for screening candidate or test compounds which are substrates of a CDHN protein or polypeptide or biologically active portion thereof. In another embodiment, the invention provides assays for screening candidate or test compounds which bind to or modulate the activity of a CDHN protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a CDHN protein or biologically active portion thereof is contacted with a test compound and the ability of the test compound to modulate CDHN activity is determined. Determining the ability of the test compound to modulate CDHN activity can be accomplished by monitoring, for example, cell aggregation, adhesion and/or motility in a cell which expresses CDHN. The cell, for example, can be of mammalian origin, e.g., an epithelial or neuronal cell. The ability of the test compound to modulate CDHN binding to a substrate or to bind to CDHN can also be determined. Determining the ability of the test compound to modulate CDHN binding to a substrate can be accomplished, for example, by coupling the CDHN substrate with a radioisotope or enzymatic label such that binding of the CDHN substrate to CDHN can be determined by detecting the labeled CDHN substrate in a complex. Alternatively, CDHN could be coupled with a radioisotope or enzymatic label to monitor the ability of a test compound to modulate CDHN binding to a CDHN substrate in a complex. Determining the ability of the test compound to bind CDHN can be accomplished, for example, by coupling the compound with a radioisotope or enzymatic label such that binding of the compound to CDHN can be determined by detecting the labeled compound in a complex. For example, compounds (e.g., CDHN substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a CDHN substrate) to interact with CDHN without the labeling of any of the interactants. For example, a microphysiometer can be used to detect the interaction of a compound with CDHN without the labeling of either the compound or the CDHN. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and CDHN.

In another embodiment, an assay is a cell-based assay comprising contacting a cell expressing a CDHN target molecule (e.g., a CDHN substrate) with a test compound and determining the ability of the test compound to modulate (e.g., stimulate or inhibit) the activity of the CDHN target molecule. Determining the ability of the test compound to modulate the activity of a CDHN target molecule can be accomplished, for example, by determining the ability of the CDHN protein to bind to or interact with the CDHN target molecule.

Determining the ability of the CDHN protein, or a biologically active fragment thereof, to bind to or interact with a CDHN target molecule can be accomplished by one of the methods described above for determining direct binding. In a preferred embodiment, determining the ability of the CDHN protein to bind to or interact with a CDHN target molecule can be accomplished by determining the activity of the target molecule. For example, the activity of the target molecule can be determined by detecting induction of a cellular response (i.e., cell proliferation, differentiation, adhesion, migration and/or signal transduction), detecting catalytic/enzymatic activity of the target on an appropriate substrate, detecting the induction of a reporter gene (comprising a target-responsive regulatory element operatively linked to a nucleic acid encoding a detectable marker, e.g., luciferase), or detecting a target-regulated cellular response.

In yet another embodiment, an assay of the present invention is a cell-free assay in which a CDHN protein or biologically active portion thereof is contacted with a test compound and the ability of the test compound to bind to the CDHN protein or biologically active portion thereof is determined. Preferred biologically active portions of the CDHN proteins to be used in assays of the present invention include fragments which participate in interactions with non-CDHN molecules, e.g., fragments with high surface probability scores (see, for example, FIGS. 2 and 8). Binding of the test compound to the CDHN protein can be determined either directly or indirectly as described above. In a preferred embodiment, the assay includes contacting the CDHN protein or biologically active portion thereof with a known compound which binds the CDHN to form an assay mixture, contacting the assay mixture with a test compound, and determining the ability of the test compound to interact with the CDHN protein, wherein determining the ability of the test compound to interact with the CDHN protein comprises determining the ability of the test compound to preferentially bind to the CDHN or biologically active portion thereof as compared to the known compound.

In another embodiment, the assay is a cell-free assay in which a CDHN protein or biologically active portion thereof is contacted with a test compound and the ability of the test compound to modulate (e.g., stimulate or inhibit) the activity of the CDHN protein or biologically active portion thereof is determined. Determining the ability of the test compound to modulate the activity of a CDHN protein can be accomplished, for example, by determining the ability of the CDHN protein to bind to a CDHN target molecule by one of the methods described above for determining direct binding. Determining the ability of the CDHN protein to bind to a CDHN target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a CDHN protein can be accomplished by determining the ability of the CDHN protein to further modulate the activity of a downstream effector of a CDHN target molecule. For example, the activity of the effector molecule on an appropriate target can be determined or the binding of the effector to an appropriate target can be determined as previously described.

In yet another embodiment, the cell-free assay involves contacting a CDHN protein or biologically active portion thereof with a known compound (e.g., a CDHN substrate) which binds the CDHN protein to form an assay mixture, contacting the assay mixture with a test compound, and determining the ability of the test compound to interact with the CDHN protein, wherein determining the ability of the test compound to interact with the CDHN protein comprises determining the ability of the CDHN protein to preferentially bind to or modulate the activity of a CDHN target protein, e.g., associate with the cytoskeleton via a CDHN substrate.

In more than one embodiment of the above assay methods of the present invention, it may be desirable to immobilize either CDHN or its target molecule to facilitate separation of complexed from uncomplexed forms of one or both of the proteins, as well as to accommodate automation of the assay. Binding of a test compound to a CDHN protein, or interaction of a CDHN protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/CDHN fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or CDHN protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of CDHN binding or activity determined using standard techniques.

Other techniques for immobilizing proteins on matrices can also be used in the screening assays of the invention. For example, either a CDHN protein or a CDHN target molecule can be immobilized utilizing conjugation of biotin and streptavidin. Biotinylated CDHN protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with CDHN protein or target molecules but which do not interfere with binding of the CDHN protein to its target molecule can be derivatized to the wells of the plate, and unbound target or CDHN protein trapped in the wells by antibody conjugation. Methods for detecting such complexes, in addition to those described above for the GST-immobilized complexes, include immunodetection of complexes using antibodies reactive with the CDHN protein or target molecule, as well as enzyme-linked assays which rely on detecting an enzymatic activity associated with the CDHN protein or target molecule.

In another embodiment, modulators of CDHN expression are identified in a method wherein a cell is contacted with a candidate compound and the expression of CDHN mRNA or protein in the cell is determined. The level of expression of CDHN mRNA or protein in the presence of the candidate compound is compared to the level of expression of CDHN mRNA or protein in the absence of the candidate compound. The candidate compound can then be identified as a modulator of CDHN expression based on this comparison. For example, when expression of CDHN mRNA or protein is greater (statistically significantly greater) in the presence of the candidate compound than in its absence, the candidate compound is identified as a stimulator of CDHN mRNA or protein expression. Alternatively, when expression of CDHN mRNA or protein is less (statistically significantly less) in the presence of the candidate compound than in its absence, the candidate compound is identified as an inhibitor of CDHN mRNA or protein expression. The level of CDHN mRNA or protein expression in the cells can be determined by methods described herein for detecting CDHN mRNA or protein.

In yet another aspect of the invention, the CDHN proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with CDHN (“CDHN binding proteins” or “CDHN-bp”) and are involved in CDHN activity. Such CDHN binding proteins are also likely to be involved in the propagation of signals by the CDHN proteins or CDHN targets as, for example, downstream elements of a CDHN-mediated signaling pathway. Alternatively, such CDHN binding proteins are likely to be CDHN inhibitors.

The two-hybrid system is based on the modular nature of most transcription factors, which consist of separable DNA-binding and activation domains. Briefly, the assay utilizes two different DNA constructs. In one construct, the gene that codes for a CDHN protein, or a biologically active portion thereof, is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a CDHN-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the CDHN protein.

In another aspect, the invention pertains to a combination of two or more of the assays described herein. For example, a modulating agent can be identified using a cell-based or a cell free assay, and the ability of the agent to modulate the activity of a CDHN protein can be confirmed in vivo, e.g., in an animal such as an animal model for cellular transformation, tumorigenesis and/or metastasis.

This invention further pertains to novel agents identified by the above-described screening assays. Accordingly, it is within the scope of this invention to further use an agent identified as described herein in an appropriate animal model. For example, an agent identified as described herein (e.g., a CDHN modulating agent, an antisense CDHN nucleic acid molecule, a CDHN-specific antibody, or a CDHN binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

### B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

### 1. Chromosome Mapping

Once the sequence (or a portion of the sequence) of a gene has been isolated, this sequence can be used to map the location of the gene on a chromosome. This process is called chromosome mapping. Accordingly, portions or fragments of the CDHN nucleotide sequences, described herein, can be used to map the location of the CDHN genes on a chromosome. The mapping of the CDHN sequences to chromosomes is an important first step in correlating these sequences with genes associated with disease.

Briefly, CDHN genes can be mapped to chromosomes by preparing PCR primers (preferably 15-25 bp in length) from the CDHN nucleotide sequences. Computer analysis of the CDHN sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the CDHN sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the CDHN nucleotide sequences to design oligonucleotide primers, sublocalization can be achieved with panels of fragments from specific chromosomes. Other mapping strategies which can similarly be used to map a CDHN sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with a CDHN gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

### 2. Tissue Typing

The CDHN sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the CDHN nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The CDHN nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1 or 4 can comfortably provide positive individual identification with a panel of perhaps 10 to 1,000 primers which each yield a noncoding amplified sequence of 100 bases. If predicted coding sequences, such as those in SEQ ID NO:3 or 6 are used, a more appropriate number of primers for positive individual identification would be 500-2,000.

If a panel of reagents from CDHN nucleotide sequences described herein is used to generate a unique identification database for an individual, those same reagents can later be used to identify tissue from that individual. Using the unique identification database, positive identification of the individual, living or dead, can be made from extremely small tissue samples.

### 3. Use of CDHN Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1 or SEQ ID NO:4 are particularly appropriate for this use as greater numbers of polymorphisms occur in the noncoding regions, making it easier to differentiate individuals using this technique. Examples of polynucleotide reagents include the CDHN nucleotide sequences or portions thereof, e.g., fragments derived from the noncoding regions of SEQ ID NO:1 or SEQ ID NO:4 having a length of at least 20 bases, preferably at least 30 bases.

The CDHN nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such CDHN probes can be used to identify tissue by species and/or by organ type.

In a similar fashion, these reagents, e.g., CDHN primers or probes can be used to screen tissue culture for contamination (i.e. screen for the presence of a mixture of different types of cells in a culture).

### C. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining CDHN protein and/or nucleic acid expression as well as CDHN activity, in the context of a biological sample (e.g., blood, serum, cells, tissue) to thereby determine whether an individual is afflicted with a disease or disorder, or is at risk of developing a disorder, associated with aberrant or unwanted CDHN expression or activity. The invention also provides for prognostic (or predictive) assays for determining whether an individual is at risk of developing a disorder associated with CDHN protein, nucleic acid expression or activity. For example, mutations in a CDHN gene can be assayed in a biological sample. Such assays can be used for prognostic or predictive purpose to thereby phophylactically treat an individual prior to the onset of a disorder characterized by or associated with CDHN protein, nucleic acid expression or activity.

Another aspect of the invention pertains to monitoring the influence of agents (e.g., drugs, compounds) on the expression or activity of CDHN in clinical trials.

These and other agents are described in further detail in the following sections.

### 1. Diagnostic Assays

An exemplary method for detecting the presence or absence of CDHN protein or nucleic acid in a biological sample involves obtaining a biological sample from a test subject and contacting the biological sample with a compound or an agent capable of detecting CDHN protein or nucleic acid (e.g., mRNA, or genomic DNA) that encodes CDHN protein such that the presence of CDHN protein or nucleic acid is detected in the biological sample. A preferred agent for detecting CDHN mRNA or genomic DNA is a labeled nucleic acid probe capable of hybridizing to CDHN mRNA or genomic DNA. The nucleic acid probe can be, for example, the CDHN nucleic acid set forth in SEQ ID NO:1, 3, 4 or 6, or the DNA insert of the plasmid deposited with ATCC as Accession Number ______, or a portion thereof, such as an oligonucleotide of at least 15, 30, 50, 100, 250 or 500 nucleotides in length and sufficient to specifically hybridize under stringent conditions to CDHN mRNA or genomic DNA. Other suitable probes for use in the diagnostic assays of the invention are described herein.

A preferred agent for detecting CDHN protein is an antibody capable of binding to CDHN protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect CDHN mRNA, protein, or genomic DNA in a biological sample in vitro as well as in vivo. For example, in vitro techniques for detection of CDHN mRNA include Northern hybridizations and in situ hybridizations. In vitro techniques for detection of CDHN protein include enzyme linked immunosorbent assays (ELISAs), Western blots, immunoprecipitations and immunofluorescence. In vitro techniques for detection of CDHN genomic DNA include Southern hybridizations. Furthermore, in vivo techniques for detection of CDHN protein include introducing into a subject a labeled anti-CDHN antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting CDHN protein, mRNA, or genomic DNA, such that the presence of CDHN protein, mRNA or genomic DNA is detected in the biological sample, and comparing the presence of CDHN protein, mRNA or genomic DNA in the control sample with the presence of CDHN protein, mRNA or genomic DNA in the test sample.

The invention also encompasses kits for detecting the presence of a CDHN in a biological sample. For example, the kit can comprise a labeled compound or agent capable of detecting a CDHN protein or mRNA in a biological sample; means for determining the amount of CDHN in the sample; and means for comparing the amount of CDHN in the sample with a standard. The compound or agent can be packaged in a suitable container. The kit can further comprise instructions for using the kit to detect CDHN protein or nucleic acid.

### 2. Prognostic Assays

The diagnostic methods described herein can furthermore be utilized to identify subjects having or at risk of developing a disease or disorder associated with aberrant or unwanted CDHN expression or activity. As used herein, the term “aberrant” includes a CDHN expression or activity which deviates from the wild type CDHN expression or activity. Aberrant expression or activity includes increased or decreased expression or activity, as well as expression or activity which does not follow the wild type developmental pattern of expression or the subcellular pattern of expression. For example, aberrant CDHN expression or activity is intended to include the cases in which a mutation in the CDHN gene causes the CDHN gene to be under-expressed or over-expressed and situations in which such mutations result in a non-functional CDHN protein or a protein which does not function in a wild-type fashion, e.g., a protein which does not interact with a CDHN substrate, or one which interacts with a non-CDHN substrate. As used herein, the term “unwanted” includes an unwanted phenomenon involved in a biological response such as cellular proliferation. For example, the term unwanted includes a CDHN expression or activity which is undesirable in a subject.

The assays described herein, such as the preceding diagnostic assays or the following assays, can be utilized to identify a subject having or at risk of developing a disorder associated with a misregulation in CDHN protein activity or nucleic acid expression, such as a central nervous system (CNS) disorder, a cardiovascular disorder, a musculoskeletal disorder, a gastrointestinal disorder, an inflammatory or immune system disorder, or a cell proliferation, growth, differentiation, adhesion, or migration disorder. Alternatively, the prognostic assays can be utilized to identify a subject having or at risk for developing a disorder associated with a misregulation in CDHN protein activity or nucleic acid expression, such as a central nervous system (CNS) disorder, a cardiovascular disorder, a musculoskeletal disorder, a gastrointestinal disorder, an inflammatory or immune system disorder, or a cell proliferation, growth, differentiation, adhesion, or migration disorder. Thus, the present invention provides a method for identifying a disease or disorder associated with aberrant or unwanted CDHN expression or activity in which a test sample is obtained from a subject and CDHN protein or nucleic acid (e.g., mRNA or genomic DNA) is detected, wherein the presence of CDHN protein or nucleic acid is diagnostic for a subject having or at risk of developing a disease or disorder associated with aberrant or unwanted CDHN expression or activity. As used herein, a “test sample” refers to a biological sample obtained from a subject of interest. For example, a test sample can be a biological fluid (e.g., cerebrospinal fluid or serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted CDHN expression or activity. For example, such methods can be used to determine whether a subject can be effectively treated with an agent for a central nervous system (CNS) disorder, a cardiovascular disorder, a musculoskeletal disorder, a gastrointestinal disorder, an inflammatory or immune system disorder, or a cell proliferation, growth, differentiation, adhesion, or migration disorder. Thus, the present invention provides methods for determining whether a subject can be effectively treated with an agent for a disorder associated with aberrant or unwanted CDHN expression or activity in which a test sample is obtained and CDHN protein or nucleic acid expression or activity is detected (e.g., wherein the abundance of CDHN protein or nucleic acid expression or activity is diagnostic for a subject that can be administered the agent to treat a disorder associated with aberrant or unwanted CDHN expression or activity).

The methods of the invention can also be used to detect genetic alterations in a CDHN gene, thereby determining if a subject with the altered gene is at risk for a disorder characterized by misregulation in CDHN protein activity or nucleic acid expression, such as a central nervous system (CNS) disorder, a cardiovascular disorder, a musculoskeletal disorder, a gastrointestinal disorder, an inflammatory or immune system disorder, or a cell proliferation, growth, differentiation, adhesion, or migration disorder. In preferred embodiments, the methods include detecting, in a sample of cells from the subject, the presence or absence of a genetic alteration characterized by at least one of an alteration affecting the integrity of a gene encoding a CDHN protein, or the mis-expression of the CDHN gene. For example, such genetic alterations can be detected by ascertaining the existence of at least one of 1) a deletion of one or more nucleotides from a CDHN gene; 2) an addition of one or more nucleotides to a CDHN gene; 3) a substitution of one or more nucleotides of a CDHN gene, 4) a chromosomal rearrangement of a CDHN gene; 5) an alteration in the level of a messenger RNA transcript of a CDHN gene, 6) aberrant modification of a CDHN gene, such as of the methylation pattern of the genomic DNA, 7) the presence of a non-wild type splicing pattern of a messenger RNA transcript of a CDHN gene, 8) a non-wild type level of a CDHN protein, 9) allelic loss of a CDHN gene, and 10) inappropriate post-translational modification of a CDHN protein. As described herein, there are a large number of assays known in the art which can be used for detecting alterations in a CDHN gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a CDHN gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a CDHN gene under conditions such that hybridization and amplification of the CDHN gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a CDHN gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in CDHN can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in CDHN can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the CDHN gene and detect mutations by comparing the sequence of the sample CDHN with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a CDHN gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type CDHN sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in CDHN cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a CDHN sequence, e.g., a wild-type CDHN sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in CDHN genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control CDHN nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a CDHN gene.

Furthermore, any cell type or tissue in which CDHN is expressed may be utilized in the prognostic assays described herein.

### 3. Monitoring of Effects During Clinical Trials

Monitoring the influence of agents (e.g., drugs) on the expression or activity of a CDHN protein (e.g., the modulation of cell proliferation, differentiation, adhesion, migration and/or signaling mechanisms) can be applied not only in basic drug screening, but also in clinical trials. For example, the effectiveness of an agent determined by a screening assay as described herein to increase CDHN gene expression, protein levels, or upregulate CDHN activity, can be monitored in clinical trials of subjects exhibiting decreased CDHN gene expression, protein levels, or downregulated CDHN activity. Alternatively, the effectiveness of an agent determined by a screening assay to decrease CDHN gene expression, protein levels, or downregulate CDHN activity, can be monitored in clinical trials of subjects exhibiting increased CDHN gene expression, protein levels, or upregulated CDHN activity. In such clinical trials, the expression or activity of a CDHN gene, and preferably, other genes that have been implicated in, for example, a CDHN-associated disorder can be used as a “read out” or markers of the phenotype of a particular cell.

For example, and not by way of limitation, genes, including CDHN, that are modulated in cells by treatment with an agent (e.g., compound, drug or small molecule) which modulates CDHN activity (e.g., identified in a screening assay as described herein) can be identified. Thus, to study the effect of agents on CDHN-associated disorders (e.g., disorders characterized by deregulated cell proliferation, differentiation, adhesion, migration and/or signaling mechanisms), for example, in a clinical trial, cells can be isolated and RNA prepared and analyzed for the levels of expression of CDHN and other genes implicated in the CDHN-associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of CDHN or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a CDHN protein, mRNA, or genomic DNA in the preadministration sample; (iii) obtaining one or more post-administration samples from the subject; (iv) detecting the level of expression or activity of the CDHN protein, mRNA, or genomic DNA in the post-administration samples; (v) comparing the level of expression or activity of the CDHN protein, mRNA, or genomic DNA in the pre-administration sample with the CDHN protein, mRNA, or genomic DNA in the post administration sample or samples; and (vi) altering the administration of the agent to the subject accordingly. For example, increased administration of the agent may be desirable to increase the expression or activity of CDHN to higher levels than detected, i.e., to increase the effectiveness of the agent. Alternatively, decreased administration of the agent may be desirable to decrease expression or activity of CDHN to lower levels than detected, i.e. to decrease the effectiveness of the agent. According to such an embodiment, CDHN expression or activity may be used as an indicator of the effectiveness of an agent, even in the absence of an observable phenotypic response.

### D. Methods of Treatment

The present invention provides for both prophylactic and therapeutic methods of treating a subject at risk of (or susceptible to) a disorder or having a disorder associated with aberrant or unwanted CDHN expression or activity, e.g., a cadherin-associated disorder such as a central nervous system (CNS) disorder, a cardiovascular disorder, a musculoskeletal disorder, a gastrointestinal disorder, an inflammatory or immune system disorder, or a cell proliferation, growth, differentiation, adhesion, or migration disorder. With regard to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the CDHN molecules of the present invention or CDHN modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

### 1. Prophylactic Methods

In one aspect, the invention provides a method for preventing in a subject, a disease or condition associated with an aberrant or unwanted CDHN expression or activity, by administering to the subject a CDHN or an agent which modulates CDHN expression or at least one CDHN activity. Subjects at risk for a disease which is caused or contributed to by aberrant or unwanted CDHN expression or activity can be identified by, for example, any or a combination of diagnostic or prognostic assays as described herein. Administration of a prophylactic agent can occur prior to the manifestation of symptoms characteristic of the CDHN aberrancy, such that a disease or disorder is prevented or, alternatively, delayed in its progression. Depending on the type of CDHN aberrancy, for example, a CDHN, CDHN agonist or CDHN antagonist agent can be used for treating the subject. The appropriate agent can be determined based on screening assays described herein.

### 2. Therapeutic Methods

Another aspect of the invention pertains to methods of modulating CDHN expression or activity for therapeutic purposes. Accordingly, in an exemplary embodiment, the modulatory method of the invention involves contacting a cell with a CDHN or agent that modulates one or more of the activities of CDHN protein activity associated with the cell. An agent that modulates CDHN protein activity can be an agent as described herein, such as a nucleic acid or a protein, a naturally-occurring target molecule of a CDHN protein (e.g., a CDHN substrate), a CDHN antibody, a CDHN agonist or antagonist, a peptidomimetic of a CDHN agonist or antagonist, or other small molecule. In one embodiment, the agent stimulates one or more CDHN activities. Examples of such stimulatory agents include active CDHN protein and a nucleic acid molecule encoding a CDHN that has been introduced into the cell. In another embodiment, the agent inhibits one or more CDHN activities. Examples of such inhibitory agents include antisense CDHN nucleic acid molecules, anti-CDHN antibodies, and CDHN inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a CDHN protein or nucleic acid molecule. In one embodiment, the method involves administering an agent (e.g., an agent identified by a screening assay described herein), or combination of agents that modulates (e.g., upregulates or downregulates) CDHN expression or activity. In another embodiment, the method involves administering a CDHN protein or nucleic acid molecule as therapy to compensate for reduced, aberrant, or unwanted CDHN expression or activity.

Stimulation of CDHN activity is desirable in situations in which CDHN is abnormally downregulated and/or in which increased CDHN activity is likely to have a beneficial effect. Likewise, inhibition of CDHN activity is desirable in situations in which CDHN is abnormally upregulated and/or in which decreased CDHN activity is likely to have a beneficial effect.

### 3. Pharmacogenomics

The CDHN molecules of the present invention, as well as agents, or modulators which have a stimulatory or inhibitory effect on CDHN activity (e.g., CDHN gene expression) as identified by a screening assay described herein can be administered to individuals to treat (prophylactically or therapeutically) CDHN-associated disorders (e.g., central nervous system (CNS) disorders, cardiovascular disorders; musculoskeletal disorders, gastrointestinal disorders, inflammatory or immune system disorders, or cell proliferation, growth, differentiation, adhesion, or migration disorders) associated with aberrant or unwanted CDHN activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a CDHN molecule or CDHN modulator as well as tailoring the dosage and/or therapeutic regimen of treatment with a CDHN molecule or CDHN modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a CDHN protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C 19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a CDHN molecule or CDHN modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a CDHN molecule or CDHN modulator, such as a modulator identified by one of the exemplary screening assays described herein.

### 4. Electronic Apparatus Readable Media and Arrays

Electronic apparatus readable media comprising CDHN sequence information is also provided. As used herein, “CDHN sequence information” refers to any nucleotide and/or amino acid sequence information particular to the CDHN molecules of the present invention, including but not limited to full-length nucleotide and/or amino acid sequences, partial nucleotide and/or amino acid sequences, polymorphic sequences including single nucleotide polymorphisms (SNPs), epitope sequences, and the like. Moreover, information “related to” said CDHN sequence information includes detection of the presence or absence of a sequence (e.g., detection of expression of a sequence, fragment, polymorphism, etc.), determination of the level of a sequence (e.g., detection of a level of expression, for example, a quantitative detection), detection of a reactivity to a sequence (e.g., detection of protein expression and/or levels, for example, using a sequence-specific antibody), and the like. As used herein, “electronic apparatus readable media” refers to any suitable medium for storing, holding, or containing data or information that can be read and accessed directly by an electronic apparatus. Such media can include, but are not limited to: magnetic storage media, such as floppy discs, hard disc storage medium, and magnetic tape; optical storage media such as compact discs; electronic storage media such as RAM, ROM, EPROM, EEPROM and the like; and general hard disks and hybrids of these categories such as magnetic/optical storage media. The medium is adapted or configured for having recorded thereon CDHN sequence information of the present invention.

As used herein, the term “electronic apparatus” is intended to include any suitable computing or processing apparatus or other device configured or adapted for storing data or information. Examples of electronic apparatus suitable for use with the present invention include stand-alone computing apparatuses; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the CDHN sequence information. A variety of software programs and formats can be used to store the sequence information on the electronic apparatus readable medium. For example, the sequence information can be represented in a word processing text file, formatted in commercially-available software such as WordPerfect and Microsoft Word, represented in the form of an ASCII file, or stored in a database application, such as DB2, Sybase, Oracle, or the like, as well as in other forms. Any number of dataprocessor structuring formats (e.g., text file or database) may be employed in order to obtain or create a medium having recorded thereon the CDHN sequence information.

By providing CDHN sequence information in readable form, one can routinely access the sequence information for a variety of purposes. For example, one skilled in the art can use the sequence information in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a CDHN associated disease or disorder or a pre-disposition to a CDHN associated disease or disorder, wherein the method comprises the steps of determining CDHN sequence information associated with the subject and based on the CDHN sequence information, determining whether the subject has a CDHN associated disease or disorder or a pre-disposition to a CDHN associated disease or disorder, and/or recommending a particular treatment for the disease, disorder, or pre-disease condition.

The present invention further provides in an electronic system and/or in a network, a method for determining whether a subject has a CDHN associated disease or disorder or a pre-disposition to a disease associated with CDHN wherein the method comprises the steps of determining CDHN sequence information associated with the subject, and based on the CDHN sequence information, determining whether the subject has a CDHN associated disease or disorder or a pre-disposition to a CDHN associated disease or disorder, and/or recommending a particular treatment for the disease, disorder or pre-disease condition. The method may further comprise the step of receiving phenotypic information associated with the subject and/or acquiring from a network phenotypic information associated with the subject.

The present invention also provides in a network, a method for determining whether a subject has a CDHN associated disease or disorder or a pre-disposition to a CDHN associated disease or disorder associated with CDHN, said method comprising the steps of receiving CDHN sequence information from the subject and/or information related thereto, receiving phenotypic information associated with the subject, acquiring information from the network corresponding to CDHN and/or a CDHN associated disease or disorder, and based on one or more of the phenotypic information, the CDHN information (e.g., sequence information and/or information related thereto), and the acquired information, determining whether the subject has a CDHN associated disease or disorder or a pre-disposition to a CDHN associated disease or disorder. The method may further comprise the step of recommending a particular treatment for the disease, disorder or pre-disease condition.

The present invention also provides a business method for determining whether a subject has a CDHN associated disease or disorder or a pre-disposition to a CDHN associated disease or disorder, said method comprising the steps of receiving information related to CDHN (e.g., sequence information and/or information related thereto), receiving phenotypic information associated with the subject, acquiring information from the network related to CDHN and/or related to a CDHN associated disease or disorder, and based on one or more of the phenotypic information, the CDHN information, and the acquired information, determining whether the subject has a CDHN associated disease or disorder or a pre-disposition to a CDHN associated disease or disorder. The method may further comprise the step of recommending a particular treatment for the disease, disorder or pre-disease condition.

The invention also includes an array comprising a CDHN sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be CDHN. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a CDHN associated disease or disorder, progression of CDHN associated disease or disorder, and processes, such a cellular transformation associated with the CDHN associated disease or disorder.

The array is also useful for ascertaining the effect of the expression of a gene on the expression of other genes in the same cell or in different cells (e.g., ascertaining the effect of CDHN expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including CDHN) that could serve as a molecular target for diagnosis or therapeutic intervention.

This invention is further illustrated by the following examples which should not be construed as limiting. The contents of all references, patents and published patent applications cited throughout this application, as well as the Figures, are incorporated herein by reference.

### EXAMPLES

### Example 1: Identification and Characterization of Human CDHN cDNAs

In this example, the identification and characterization of the genes encoding human CDHN-1 (clone Fbh57798) and CDHN-2 (clone Fbh57809) is described.

### Isolation of the CDHN cDNAs

The invention is based, at least in part, on the discovery of human genes encoding novel proteins, referred to herein as CDHN-1 and CDHN-2. The entire sequences of human clones Fbh57798 and Fbh57809 were determined and found to contain open reading frames termed human “CDHN-1” and “CDHN-2”, respectively.

The nucleotide sequence encoding the human CDHN-1 protein is shown in FIG. 1 and is set forth as SEQ ID NO:1. The protein encoded by this nucleic acid comprises about 924 amino acids and has the amino acid sequence shown in FIG. 1 and set forth as SEQ ID NO:2. The coding region (open reading frame) of SEQ ID NO:1 is set forth as SEQ ID NO:3.

The nucleotide sequence encoding the human CDHN-2 protein is shown in FIG. 7 and is set forth as SEQ ID NO:4. The protein encoded by this nucleic acid comprises about 830 amino acids and has the amino acid sequence shown in FIG. 7 and set forth as SEQ ID NO:5. The coding region (open reading frame) of SEQ ID NO:4 is set forth as SEQ ID NO:6.

Clones Fbh57798 and Fbh57809, comprising the coding region of human CDHN-1 and CDHN-2, respectively, were deposited with the American Type Culture Collection (ATCC®), 10801 University Boulevard, Manassas, Va. 20110-2209, on ______, and assigned Accession No.______

### Analysis of the Human CDHN Molecules

The amino acid sequences of human CDHN-1 and CDHN-2 were analyzed using the program PSORT (http://www. psort.nibb.acjp) to predict the localization of the proteins within the cell. This program assesses the presence of different targeting and localization amino acid sequences within the query sequence. The results of the analyses show that human CDHN-1 (SEQ ID NO:2) may be localized to the mitochondria, to the endoplasmic reticulum, to the nucleus, or to the cytoplasm. The results of the analyses further show that human CDHN-2 (SEQ ID NO:5) may be localized to the cytoplasm, to the nucleus, to the mitochondria, to the Golgi, to the endoplasmic reticulum, to secretory vesicles, or to peroxisomes.

The amino acid sequences of human CDHN-1 and CDHN-2 were also analyzed by the SignalP program (Henrik, et al. (1997) Protein Engineering 10: 1-6) for the presence of a signal peptide. These analyses revealed the presence of a signal peptide in the amino acid sequence of CDHN-1 (SEQ ID NO:2) from residues 1-33, and a signal peptide in the amino acid sequence of CDHN-2 (SEQ ID NO:5) from amino acid residues 1-21.

Searches of the amino acid sequences of CDHN-1 and CDHN-2 were performed against the Memsat database (FIGS. 3 and 9). These searches resulted in the identification of five transmembrane domains in the amino acid sequence of human CDHN-1 (SEQ ID NO:2) at about residues 19-35, 42-59, 298-315, 369-393 and 863-886 in the native moleule, and the identification of four transmembrane domains in the amino acid sequence of the predicted mature CDHN-1 protein at about residues 8-26, 265-282, 336-360 and 830-853 (FIG. 3). These searches further identified three transmembrane domains in the amino acid sequence of human CDHN-2 (SEQ ID NO:5) at about residues 540-557, 571-588 and 789-813 in the native moleule, and about residues 519-536, 550-567 and 768-792 of the predicted mature protein (FIG. 9).

Searches were performed against the Prosite database, and resulted in the identification of several possible glycosylation sites within the human CDHN proteins. For example, N-linked glycosylation sites were identified at about residues 108-111, 299-302, 305-308, 653-656, 721-724, 776-779, 817-820 and 822-825 of human CDHN-1 (SEQ ID NO:2), as well as at about residues 519-522, 604-607 and 724-727 of human CDHN-2 (SEQ ID NO:5). These searches further identified putative phosphorylation sites within the human CDHN proteins. Protein kinase C phosphorylation sites were identified at about amino acid residues 12-14, 219-221, 333-335, 366-368, 428-430, 464-466, 581-583, 609-611, 662-664 698-700, 767-769 and 850-852, casein kinase II phosphorylation sites were identified at about residues 44-47, 57-60, 82-85, 116-119, 144-147, 362-365, 428-431, 516-519, 533-536, 568-571, 601-604, 635-638, 778-781 and 824-827, and tyrosine phosphorylation sites were identified at about residues 37-43, 430-436, 572-580 and 796-802 of human CDHN-1 (SEQ ID NO:2). Furthermore, protein kinase C phosphorylation sites were identified at about residues 3-5, 597-599, 643-645, and 679-681, and casein kinase II phosphorylation sites were identified at about amino acid residues 153-156, 199-202, 234-237, 266-269, 313-316, 339-342, 361-364, 433-436, 460-463, 477-480 and 535-538, of human CDHN-2 (SEQ ID NO:5). The searches also identified the presence of N-myristoylation site motifs at about amino acid residues 48-53, 101-106, 129-134, 309-314, 377-382, 665-670, 690-695, 734-739 and 881-886 of human CDHN-1 (SEQ ID NO:2), and at about amino acid residues 140-145, 159-164, 354-359, 369-374, 426-431, 468-473, 627-632, 647-652, 685-690 and 790-795 of human CDHN-2 (SEQ ID NO:5). In addition, these searches identified the presence of cadherins extracellular repeated domain signature motifs at about amino acid residues 170-180, 281-291, 496-506, 600-610 and 703-713 of human CDHN-1 (SEQ ID NO:2), and at about amino acid residues 326-336 of human CDHN-2 (SEQ ID NO:5). Furthermore, the search identified a leucine zipper pattern at about amino acid residues 796-817 of human CDHN-2 (SEQ ID NO:5).

Searches of the amino acid sequences of CDHN-1 and CDHN-2 were also performed against the HMM (PFAM) database (FIGS. 4 and 10). This search resulted in the identification of “cadherin” domains in the amino acid sequence of CDHN-1 (SEQ ID NO:2) at about residues 187-284, 298-390, 513-603, 617-706, and 724-817. This search also resulted in the identification of “cadherin” domains at about residues 27-119, 133-234, 244-329, 343-442, 457-558 and 571-659 of human CDHN-2 (SEQ ID NO:5).

Searches of the amino acid sequences of CDHN-1 and CDHN-2 were also performed against the HMM (SMART) database (FIGS. 5 and 11). This search resulted in the identification of “CA” domains in the amino acid sequence of CDHN-1 (SEQ ID NO:2) at about residues 205-291, 315-397, 427-506, 530-610, 634-713 and 740-824. This search also resulted in the identification of “CA” domains at about residues 47-126, 150-243, 260-336, 360-449, 474-563 and 585-663 of human CDHN-2 (SEQ ID NO:5).

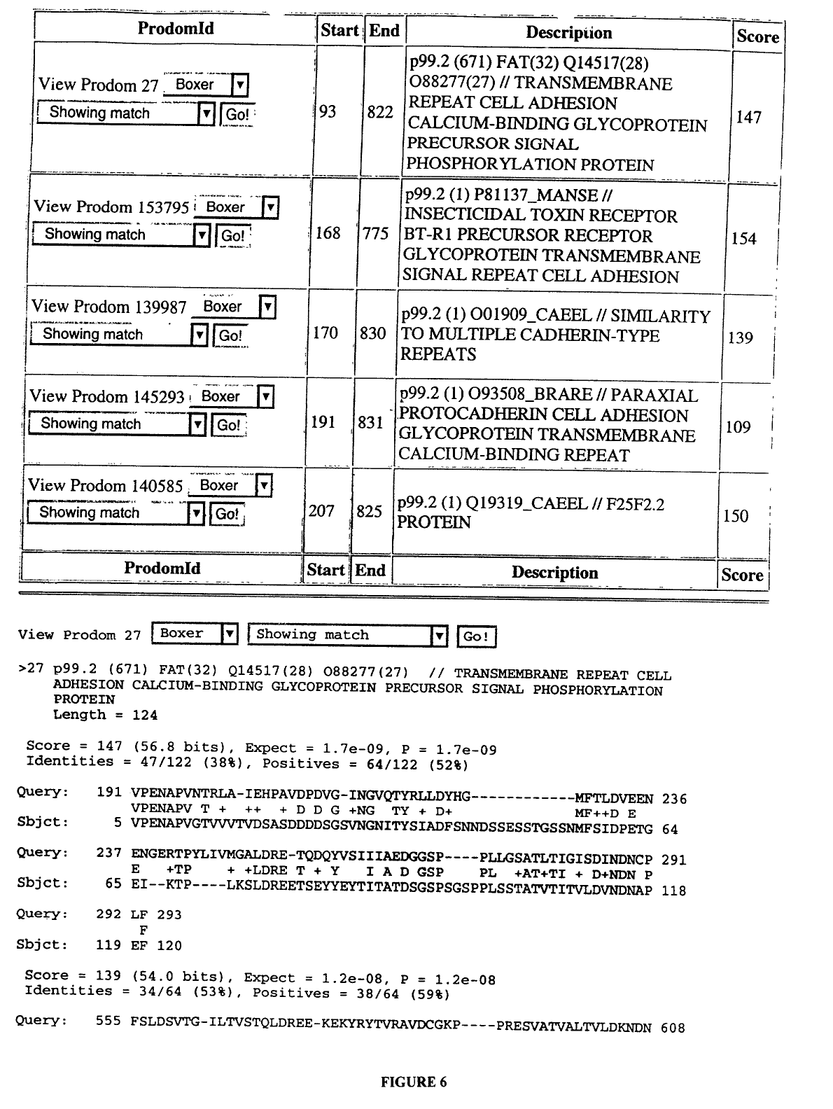

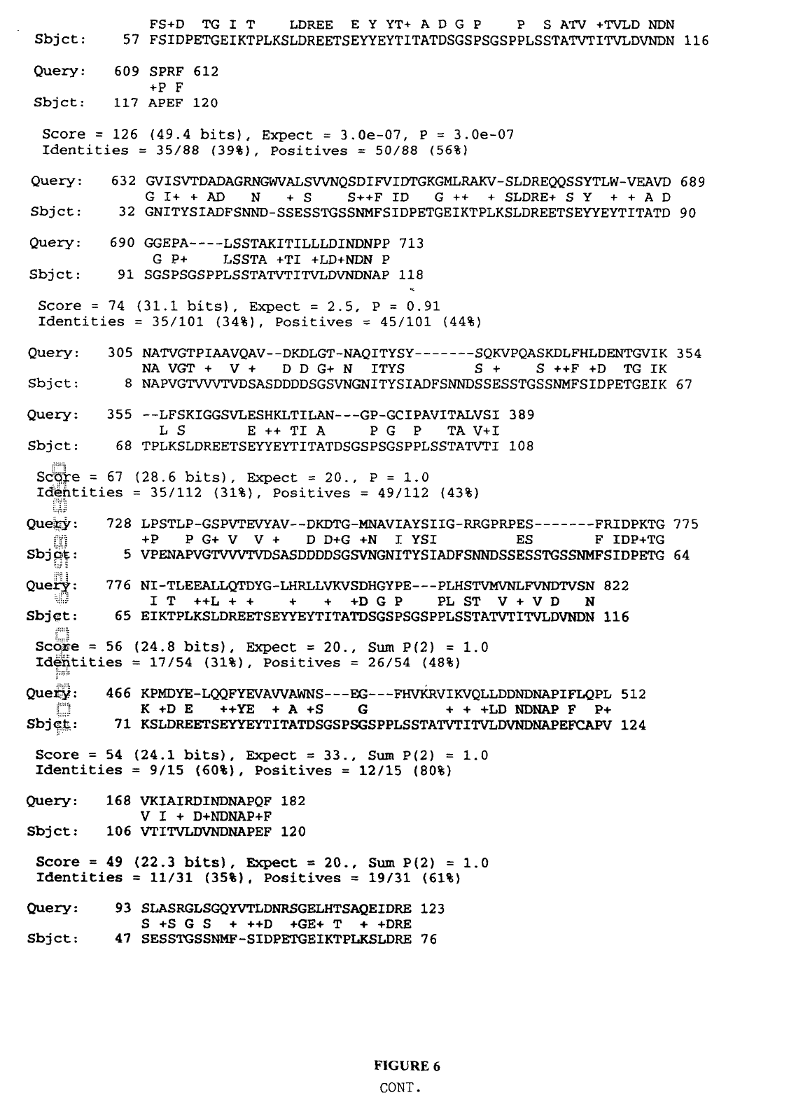

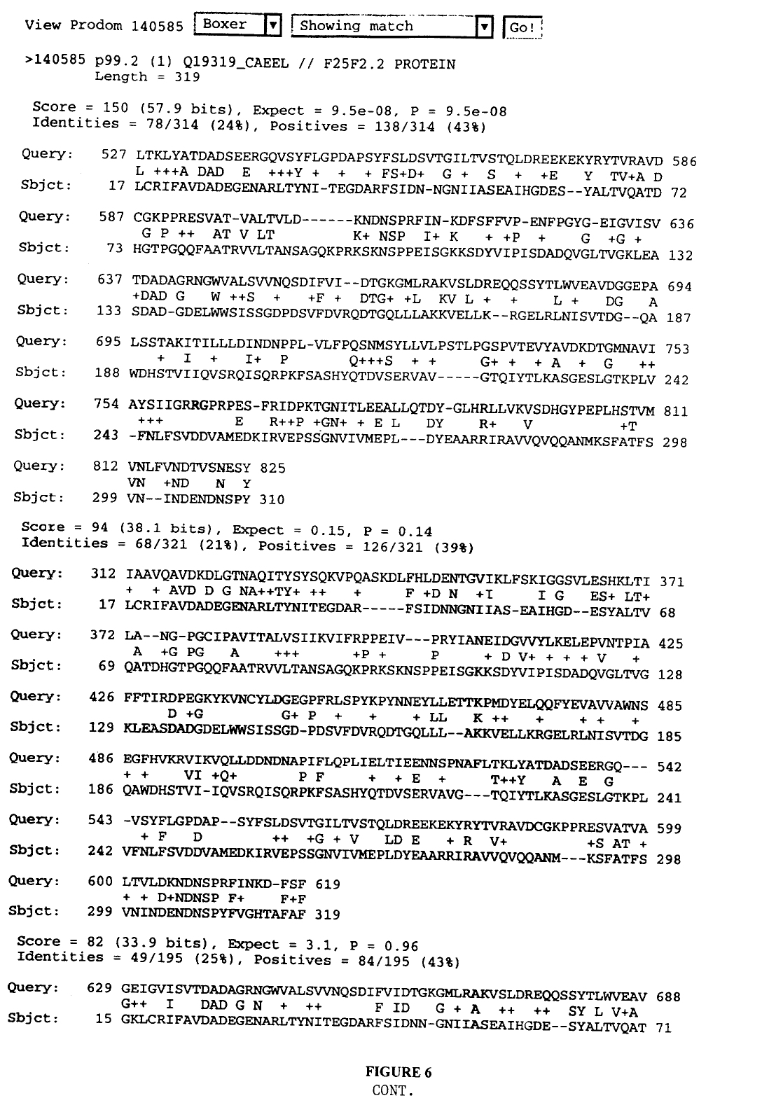

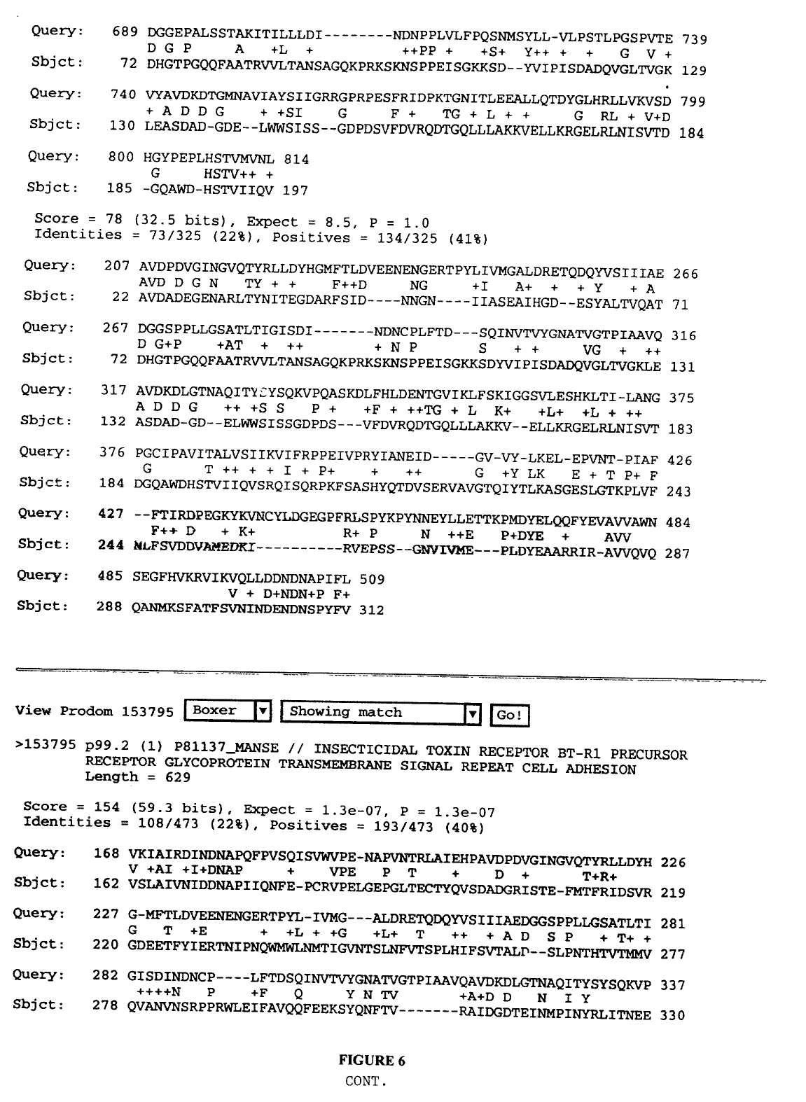

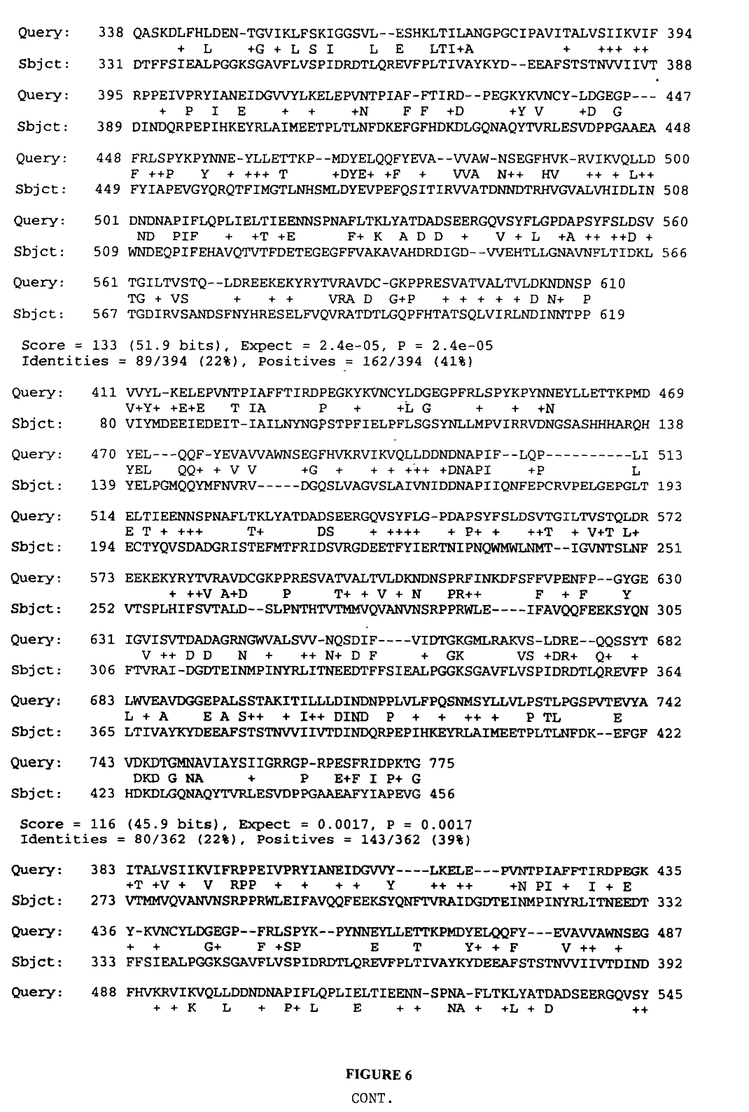

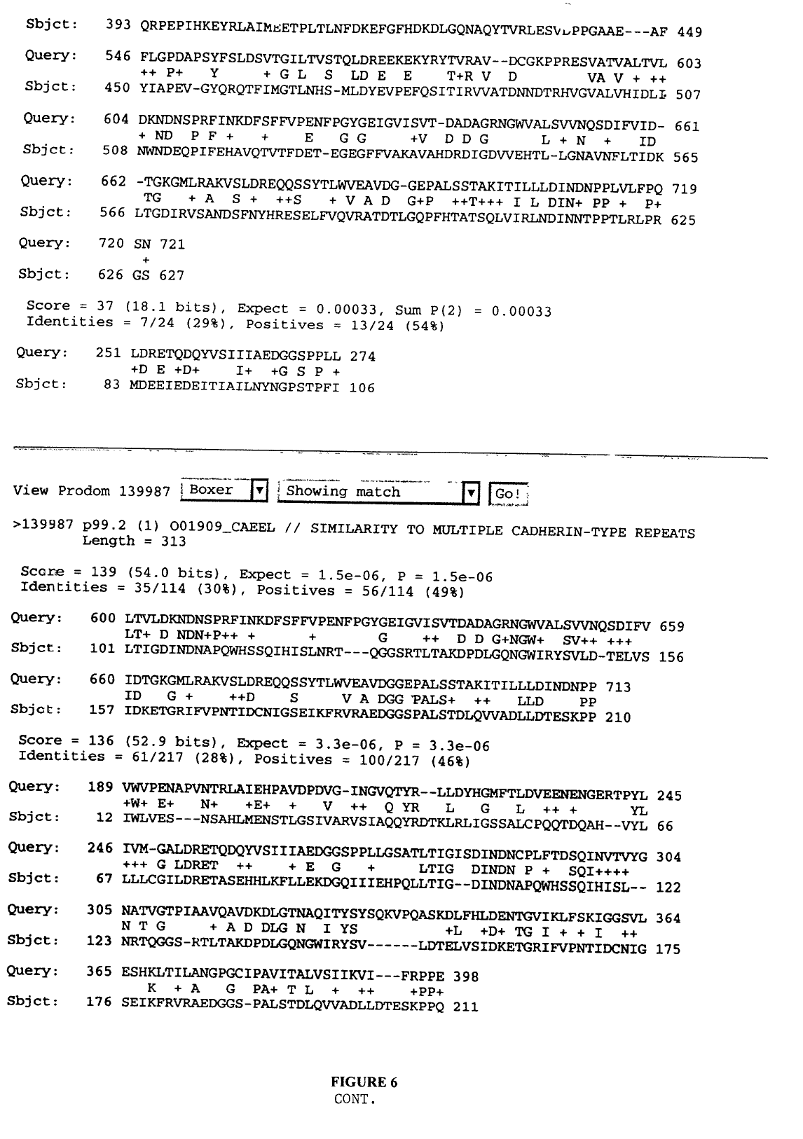

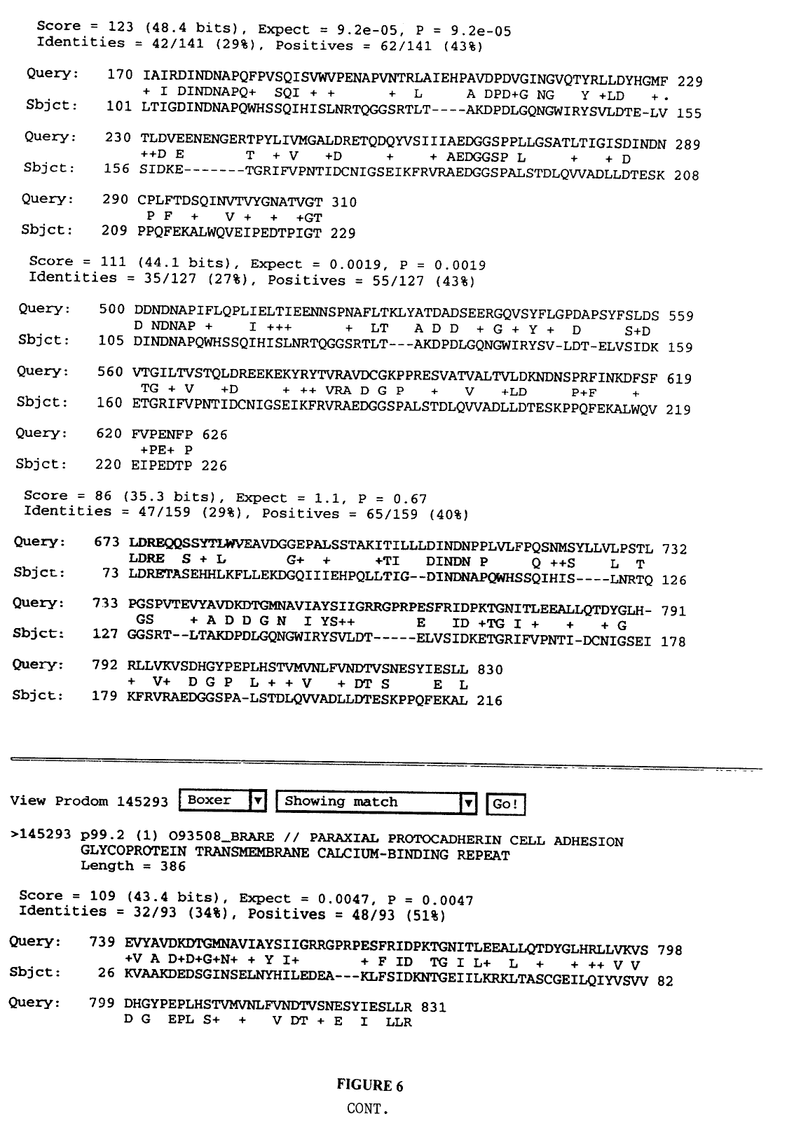

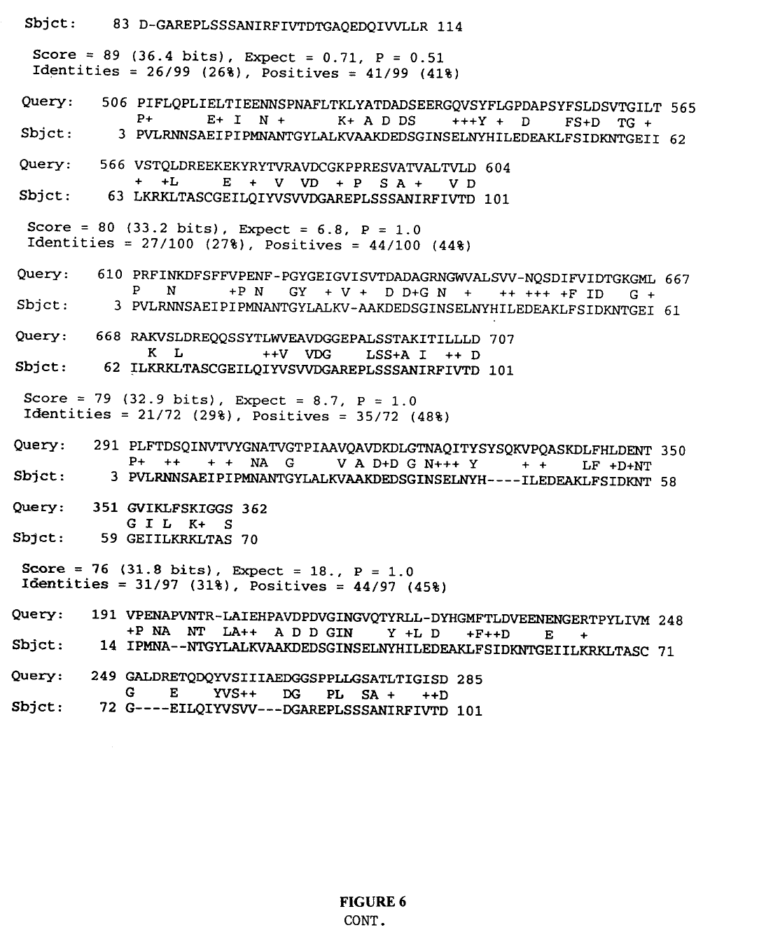

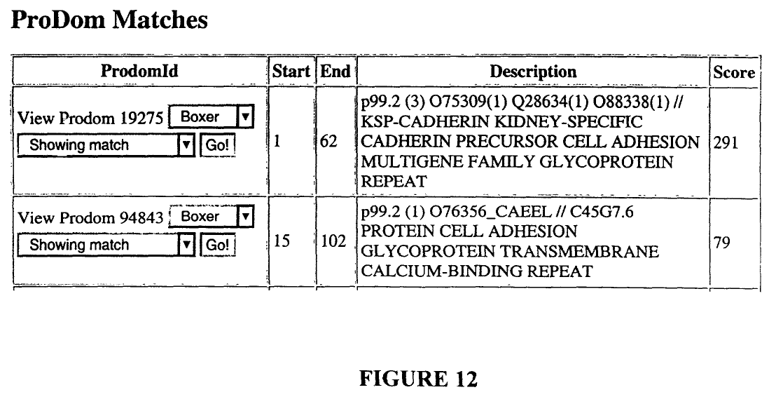

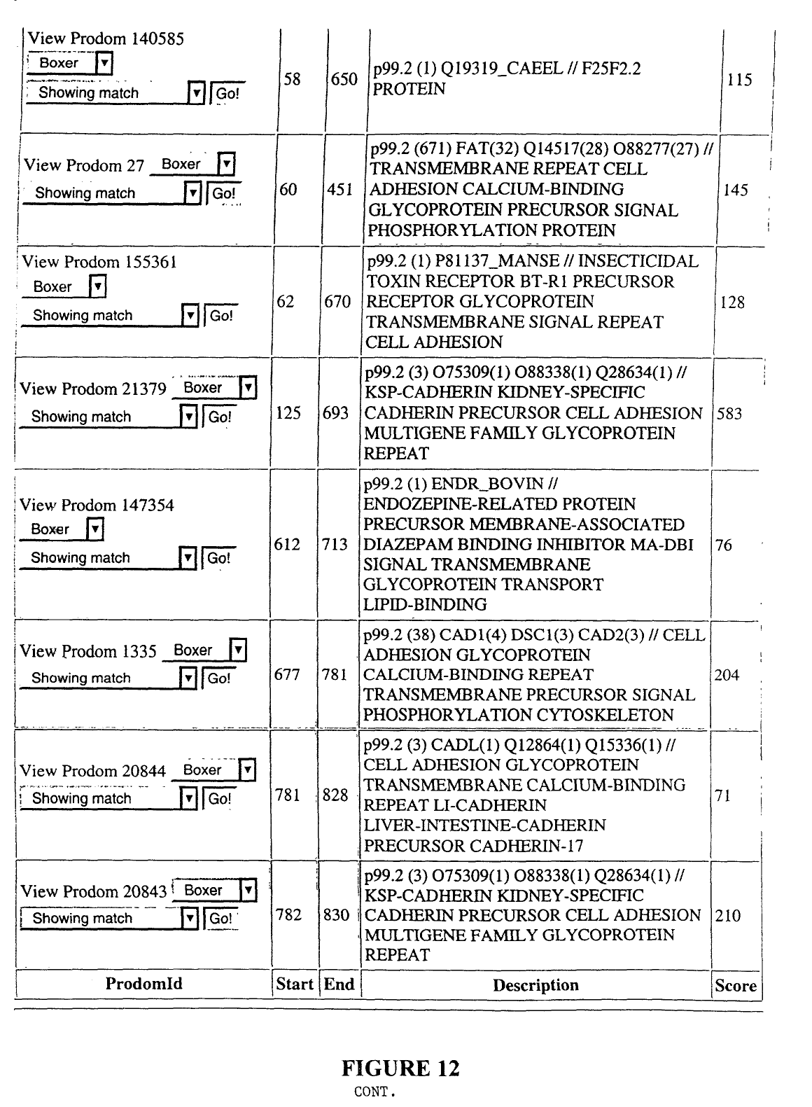

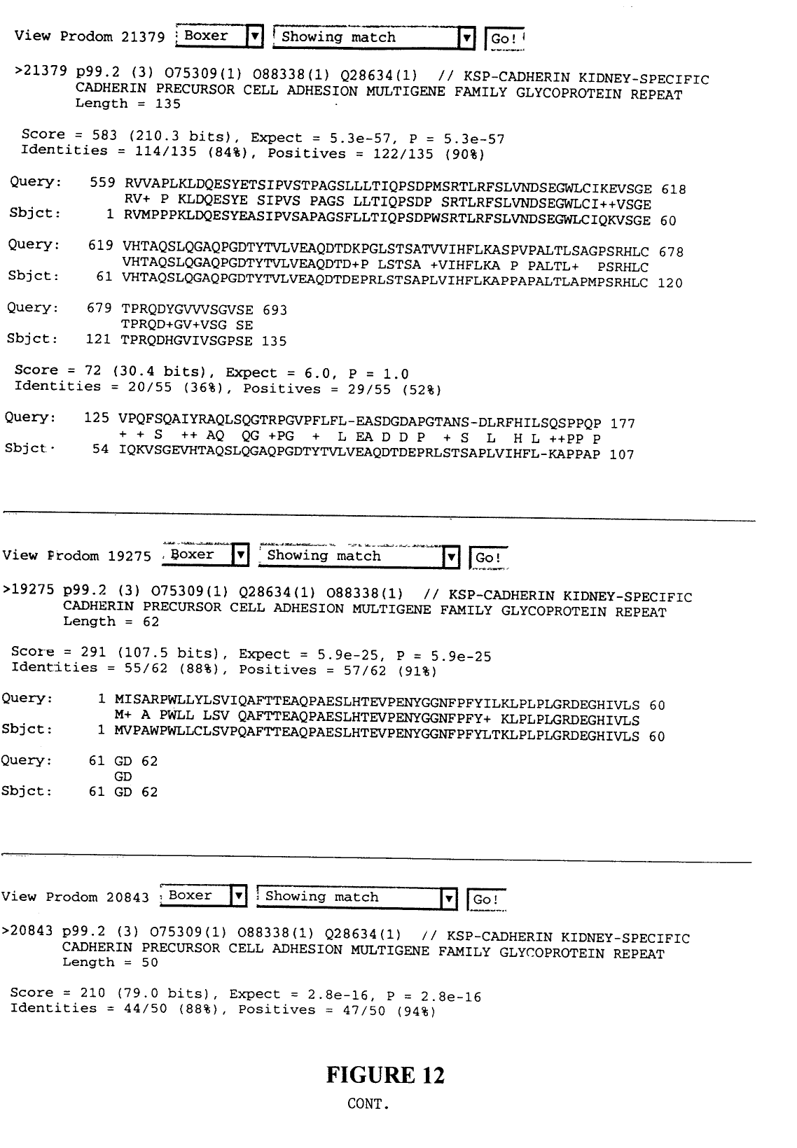

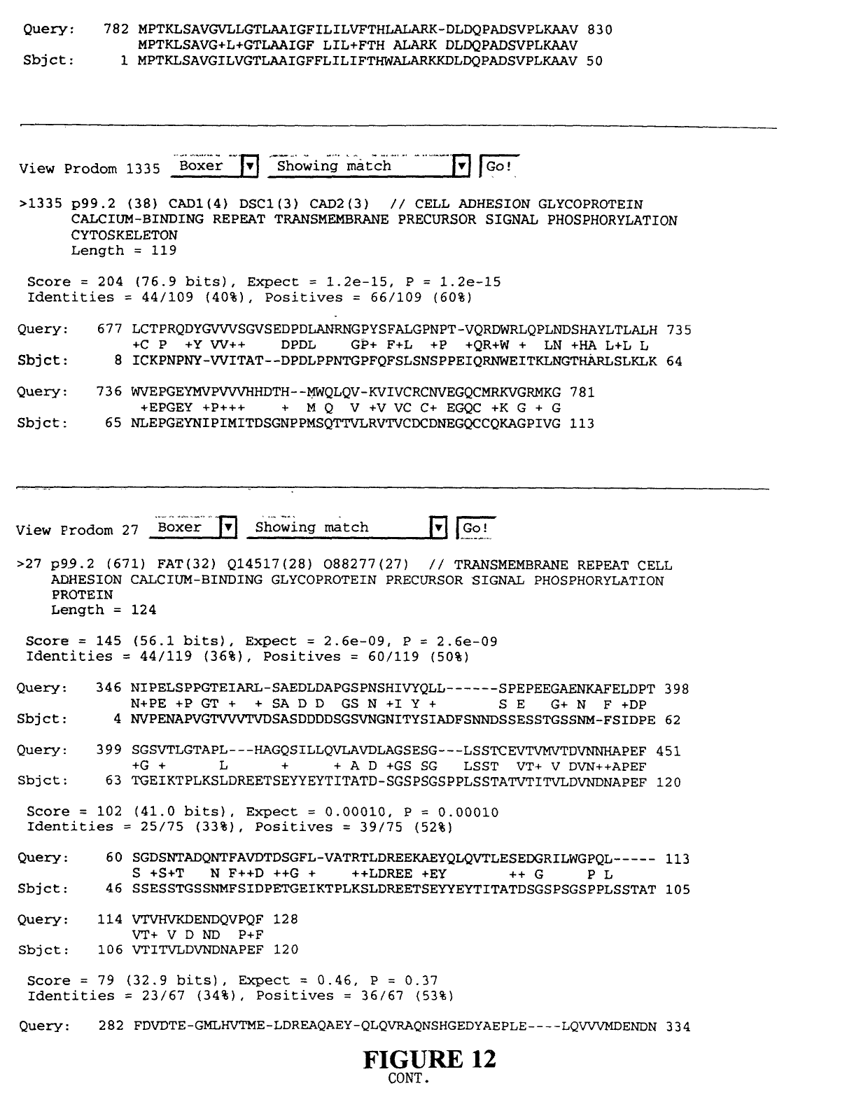

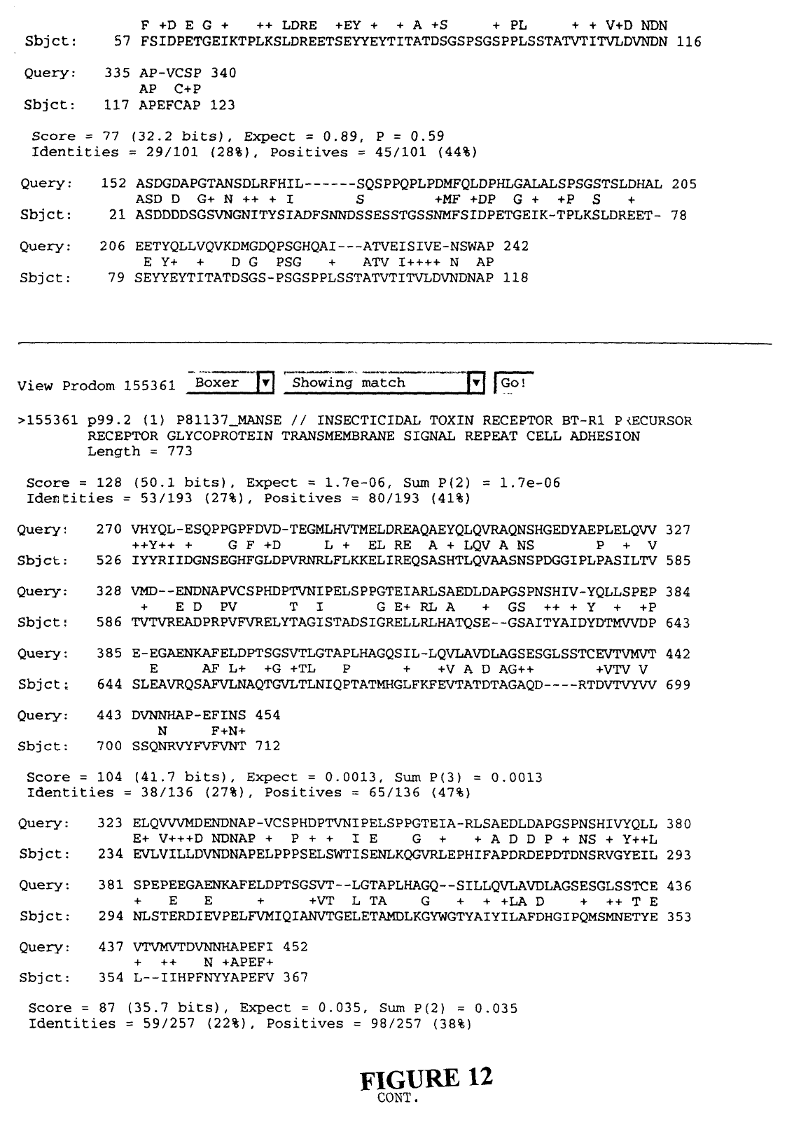

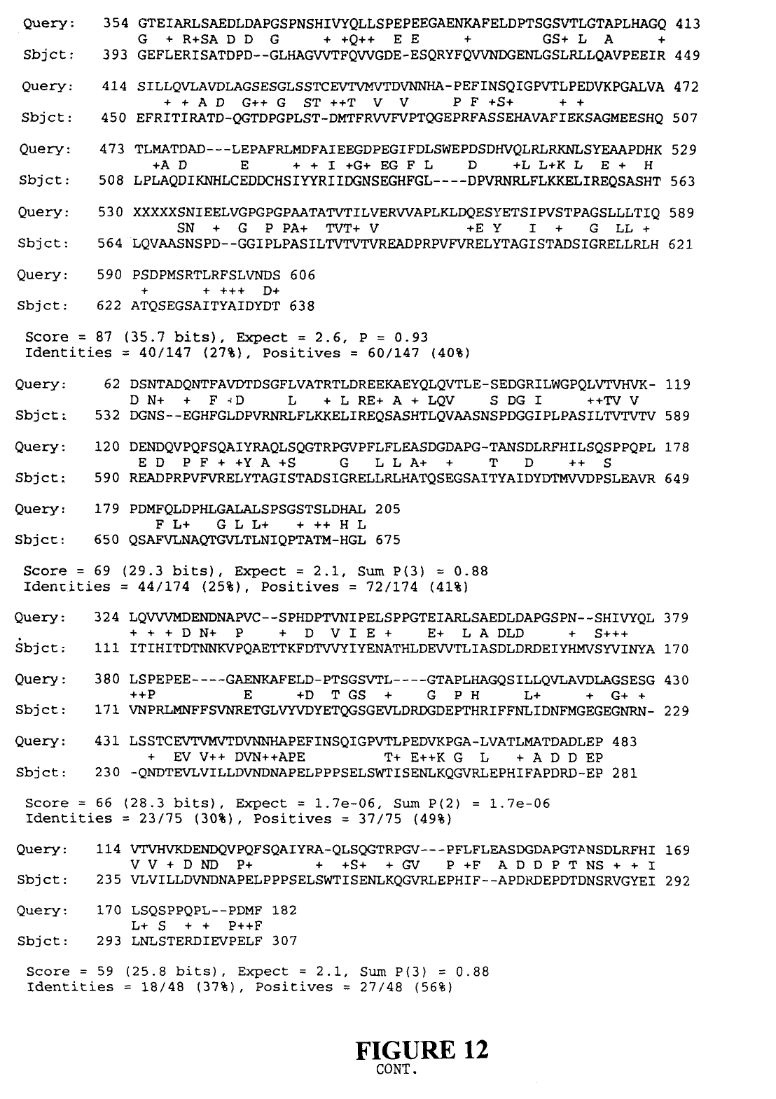

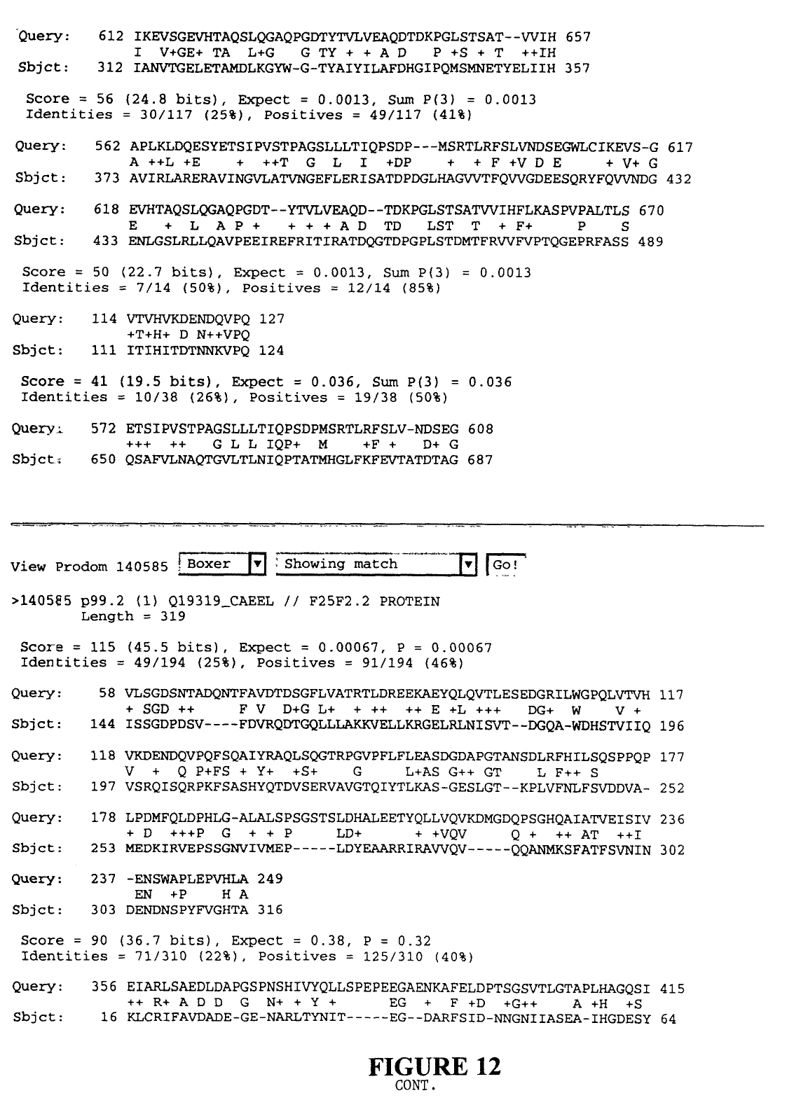

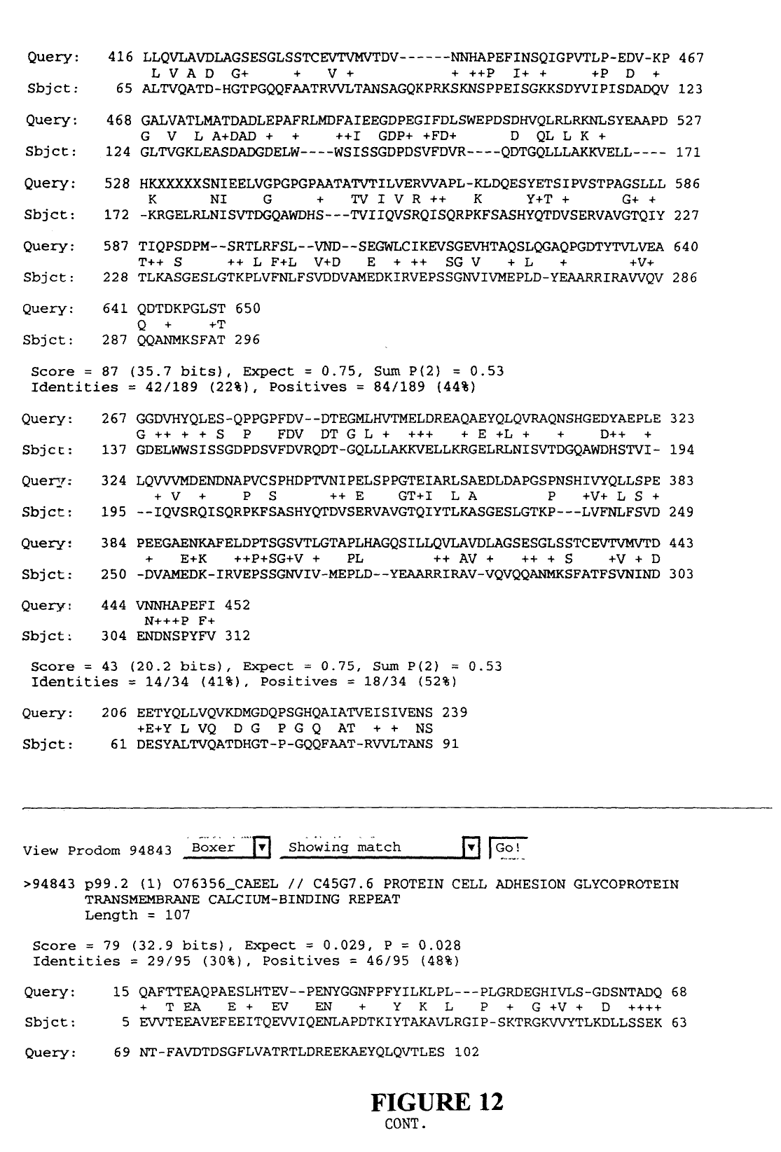

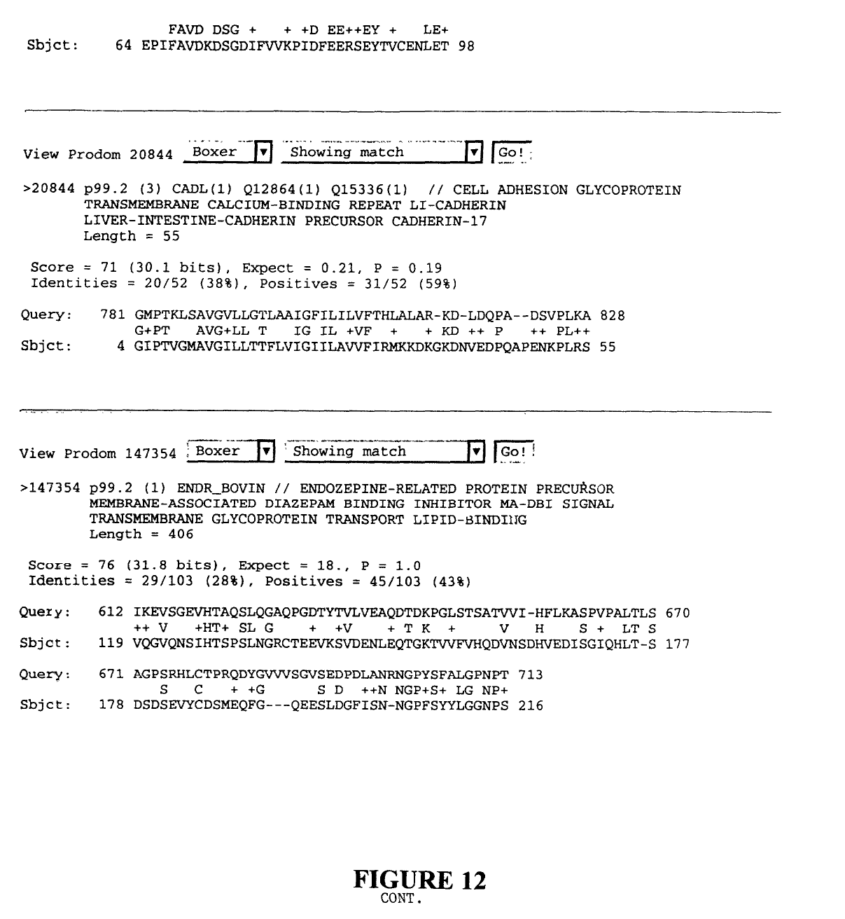

Searches of the amino acid sequences of CDHN-1 and CDHN-2 were also performed against the ProDom database (FIGS. 6 and 12). These searches resulted in the local alignment of the human CDHN-1 protein (SEQ ID NO:2) with p99.2 (671) FAT(32) Q14517(28)O 88277(27) over amino acid residues 191-293 [score=147], over amino acid residues 555-612 [score=139], over amino acid residues 632-713 [score 126], over amino acid residues 305-389 [score=74], over amino acid residues 728-822 [score=67], over amino acid residues 466-512 [score=56], over amino acid residues 168-182 [score=54], and over amino acid residues 93-123 [score=49]. These searches also resulted in the local alignment of CDHN-1 with p99.2 (1) Q19319_CAEEL over amino acid residues 527-825 [score=150], over amino acid residues 312-619 [score=94], over amino acid residues 629-814 [score=82], and over amino acid residues 207-509 [score=78]. In addition, these searches resulted in the local alignment of CDHN-1 with p99.2 (1) P81137_MANSE over amino acid residues 168-610 [score=154], over amino acid residues 411-775 [score=133], over amino acid residues 383-721 [score=116], and over amino acid residues 251-274 [score=37]. Furthermore, these searches resulted in the local alignment of CDHN-1 with p99.2 (1) O01909_CAEEL over amino acid residues 600-713 [score=139], over amino acid residues 189-398 [score=136], over amino acid residues 170-310 [score=123], over amino acid residues 500-626 [score=111], and over amino acid residues 673-830 [score=86]; and with p99.2 (1) O93508_BRARE over amino acid residues 739-831 [score=109], over amino acid residues 506-604 [score=89], over amino acid residues 610-707 [score=80], over amino acid residues 291-362 [score=79], and over amino acid residues 191-285 [score=76].

These searches resulted in the local alignment of the human CDHN-2 protein (SEQ ID NO:5) with p99.2 (3) O75309(1) O88338(1) Q28634(1) over amino acid residues 559-693 [score=583], and over amino acid residues 125-177 [score=72]. These searches also resulted in the local alignment of CDHN-2 with p99.2 (3) O75309(1) Q28634(1) O88338(1) over amino acid residues 1-62 [score=291]. In addition, these searches resulted in the local alignment of CDHN-2 with p99.2 (3) O75309 O88338(1) Q28634(1) over amino acid residues 782-830 [score=210]; and with p99.2(38) CAD1(4) DSC1(3) CAD2(3acid residues 677-781 [score=204]. These searches resulted in the local alignment of CDHN-2 with p99.2 (671) FAT(32) Q14517(28) O88277(27) over amino acid residues 346-451 [score=145], over amino acid residues 60-128 [score=102], over amino acid residues 282-340 [score=79], and over amino acid residues 152-242 [score=77]. Furthermore, these searches resulted in the local alignment of CDHN-2 with p99.2 (1) P81137_MANSE over amino acid residues 270-454 [score=128], over amino acid residues 323-452 [score=104], over amino acid residues 354-606 [score=87], over amino acid residues 62-205 [score=87], and over amino acid residues 324-483 [score=69], over amino acid residues 114-182 [score=66], over amino acid residues 612-657 [score=59], over amino acid residues 562-670 [score=56], over amino acid residues 114-127 [score=50], and over amino acid residues 572-608 [score=41]. These searches also resulted in the local alignment of CDHN-2 with p99.2 (1) Q19319 CAEEL over amino acid residues 58-249 [score=115], over amino acid residues 356-650 [score=90], over amino acid residues 267-452 [score=87], and over amino acid residues 206-239 [score=43]. In addition, these searches resulted in the local alignment of CDHN-2 with p99.2(1) O76356_CAEEL over amino acid residues 15-102 [score=79]; with p99.2 (3) CADL(1) Q12864(1) Q15336(1) over amino acid residues 781-828 [score=71]; and with p99.2 (1) ENDR_BOVIN over amino acid residues 612-713 [score=76].

### Example 2: Expression of Recombinant CDHN Protein in Bacterial Cells

In this example, CDHN is expressed as a recombinant glutathione-S-transferase (GST) fusion polypeptide in E. coli and the fusion polypeptide is isolated and characterized. Specifically, CDHN is fused to GST and this fusion polypeptide is expressed in E. coli, e.g., strain PEB 199. Expression of the GST-CDHN fusion protein in PEB 199 is induced with IPTG. The recombinant fusion polypeptide is purified from crude bacterial lysates of the induced PEB 199 strain by affinity chromatography on glutathione beads. Using polyacrylamide gel electrophoretic analysis of the polypeptide purified from the bacterial lysates, the molecular weight of the resultant fusion polypeptide is determined.

### Example 3: Expression of Recombinant CDHN Protein in COS Cells

To express the CDHN gene in COS cells, the pcDNA/Amp vector by Invitrogen Corporation (San Diego, Calif.) is used. This vector contains an SV40 origin of replication, an ampicillin resistance gene, an E coli replication origin, a CMV promoter followed by a polylinker region, and an SV40 intron and polyadenylation site. A DNA fragment encoding the entire CDHN protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the CDHN DNA sequence is amplified by PCR using two primers. The 5′ primer contains the restriction site of interest followed by approximately twenty nucleotides of the CDHN coding sequence starting from the initiation codon; the 3′ end sequence contains complementary sequences to the other restriction site of interest, a translation stop codon, the HA tag or FLAG tag and the last 20 nucleotides of the CDHN coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the CDHN gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the CDHN-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the CDHN polypeptide is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA-specific monoclonal antibody. Precipitated polypeptides are then analyzed by SDS-PAGE.

Alternatively, DNA containing the CDHN coding sequence is cloned directly into the polylinker of the pCDNA/Amp vector using the appropriate restriction sites. The resulting plasmid is transfected into COS cells in the manner described above, and the expression of the CDHN polypeptide is detected by radiolabelling and immunoprecipitation using a CDHN specific monoclonal antibody.

### Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. An isolated nucleic acid molecule selected from the group consisting of:
(a) a nucleic acid molecule comprising the nucleotide sequence set forth in SEQ ID NO:1 or 3; and
(b) a nucleic acid molecule comprising the nucleotide sequence set forth in SEQ ID NO:4 or 6.

2. An isolated nucleic acid molecule which encodes a polypeptide comprising the amino acid sequence set forth in SEQ ID NO:2 or 5.

3. An isolated nucleic acid molecule comprising the nucleotide sequence contained in the plasmid deposited with ATCC® as Accession Number ______.

4. An isolated nucleic acid molecule which encodes a naturally occurring allelic variant of a polypeptide comprising the amino acid sequence set forth, in SEQ ID NO:2 or 5.

5. An isolated nucleic acid molecule selected from the group consisting of:
a) a nucleic acid molecule comprising a nucleotide sequence which is at least 60% identical to the nucleotide sequence of SEQ ID NO:1, 3, 4 or 6, or a complement thereof;
b) a nucleic acid molecule comprising a fragment of at least 50 nucleotides of a nucleic acid comprising the nucleotide sequence of SEQ ID NO:1, 3, 4 or 6, or a complement thereof;
c) a nucleic acid molecule which encodes a polypeptide comprising an amino acid sequence at least about 60% identical to the amino acid sequence of SEQ ID NO:2 or 5;and
d) a nucleic acid molecule which encodes a fragment of a polypeptide comprising the amino acid sequence of SEQ ID NO:2 or 5, wherein the fragment comprises at least 15 contiguous amino acid residues of the amino acid sequence of SEQ ID NO:2 or 5.

6. An isolated nucleic acid molecule which hybridizes to the nucleic acid molecule of any one of claims 1, 2, 3, 4, or 5 under stringent conditions.

7. An isolated nucleic acid molecule comprising a nucleotide sequence which is complementary to the nucleotide sequence of the nucleic acid molecule of any one of claims 1,2,3,4, or 5.

8. An isolated nucleic acid molecule comprising the nucleic acid molecule of any one of claims 1, 2, 3, 4, or 5, and a nucleotide sequence encoding a heterologous polypeptide.

9. A vector comprising the nucleic acid molecule of any one of claims 1, 2, 3, 4, or 5.

10. The vector of claim 9, which is an expression vector.

11. A host cell transfected with the expression vector of claim 10.

12. A method of producing a polypeptide comprising culturing the host cell of claim 11 in an appropriate culture medium to, thereby, produce the polypeptide.

13. An isolated polypeptide selected from the group consisting of:
a) a fragment of a polypeptide comprising the amino acid sequence of SEQ ID NO:2 or 5, wherein the fragment comprises at least 15 contiguous amino acids of SEQ ID NO:2 or 5;
b) a naturally occurring allelic variant of a polypeptide comprising the amino acid sequence of SEQ ID NO:2 or 5, wherein the polypeptide is encoded by a nucleic acid molecule which hybridizes to a nucleic acid molecule consisting of SEQ ID NO:1, 3, 4 or 6 under stringent conditions;
c) a polypeptide which is encoded by a nucleic acid molecule comprising a nucleotide sequence which is at least 60% identical to a nucleic acid comprising the nucleotide sequence of SEQ ID NO:1, 3, 4 or 6;
d) a polypeptide comprising an amino acid sequence which is at least 60% identical to the amino acid sequence of SEQ ID NO:2 or 5.

14. The isolated polypeptide of claim 13 comprising the amino acid sequence of SEQ ID NO:2 or 5.

15. The polypeptide of claim 13, further comprising heterologous amino acid sequences.

16. An antibody which selectively binds to a polypeptide of claim 13.

17. A method for detecting the presence of a polypeptide of claim 13 in a sample comprising:
a) contacting the sample with a compound which selectively binds to the polypeptide; and
b) determining whether the compound binds to the polypeptide in the sample to thereby detect the presence of a polypeptide of claim 13 in the sample.

18. The method of claim 17, wherein the compound which binds to the polypeptide is an antibody.

19. A kit comprising a compound which selectively binds to a polypeptide of claim 13 and instructions for use.

20. A method for detecting the presence of a nucleic acid molecule of any one of claims 1, 2, 3, 4, or 5 in a sample comprising:
a) contacting the sample with a nucleic acid probe or primer which selectively hybridizes to the nucleic acid molecule; and
b) determining whether the nucleic acid probe or primer binds to a nucleic acid molecule in the sample to thereby detect the presence of a nucleic acid molecule of any one of claims 1, 2, 3, 4, or 5 in the sample.

21. The method of claim 20, wherein the sample comprises mRNA molecules and is contacted with a nucleic acid probe.

22. A kit comprising a compound which selectively hybridizes to a nucleic acid molecule of any one of claims 1, 2, 3, 4, or 5 and instructions for use.

23. A method for identifying a compound which binds to a polypeptide of claim 13 comprising:
a) contacting the polypeptide, or a cell expressing the polypeptide with a test compound; and
b) determining whether the polypeptide binds to the test compound.

24. The method of claim 23, wherein the binding of the test compound to the polypeptide is detected by a method selected from the group consisting of:
a) detection of binding by direct detection of test compound/polypeptide binding;
b) detection of binding using a competition binding assay; and
c) detection of binding using an assay for CDHN activity.

25. A method for modulating the activity of a polypeptide of claim 13 comprising contacting the polypeptide or a cell expressing the polypeptide with a compound which binds to the polypeptide in a sufficient concentration to modulate the activity of the polypeptide.

26. A method for identifying a compound which modulates the activity of a polypeptide of claim 13 comprising:
a) contacting a polypeptide of claim 13 with a test compound; and
b) determining the effect of the test compound on the activity of the polypeptide to thereby identify a compound which modulates the activity of the polypeptide.
